(19) United States
(12) Patent Application Publication
(10) Pub. No.: US 2025/0262308 A1
(43) Pub. Date: Aug. 21, 2025

(54) Compositions and Methods for Modulating Dopamine Receptor Activity

(71) Applicant: The Regents of the University of California, Oakland, CA (US)

(72) Inventor: Ehud Y. Isacoff, Berkeley, CA (US)

(21) Appl. No.: 18/857,145

(22) PCT Filed: Apr. 27, 2023
(86) PCT No.: PCT/US2023/066281

§ 371 Date: Oct. 15, 2024

Related U.S. Application Data
(60) Provisional application No. 63/340,740, filed on May 11, 2022.

(51) Int. Cl. A61K 47/54; A61K 41/00; A61P 25/16

(52) U.S. Cl. CPC A61K 47/545; A61K 41/00; A61P 25/16

(57) ABSTRACT

The present disclosure provides conjugates and systems for modulating the activity of a ligand-binding polypeptide such as a D2 dopamine receptor. Such conjugates comprising an affinity agent, a linker; a photoisomerizable group and a ligand that binds to a target ligand-binding polypeptide. The present disclosure provides methods of modulating the activity of a D2 dopamine receptor. The present disclosure provides methods of treating Parkinson's disease in an individual.

### CROSS-REFERENCE

This application claims the benefit of U.S. Provisional Patent Application No. 63/340,740, filed May 11, 2022, which application is incorporated herein by reference in its entirety.

### INCORPORATION-BY-REFERENCE OF MATERIAL ELECTRONICALLY SUBMITTED

A Sequence Listing is provided herewith as a Sequence Listing XML, “BERK-469WO_SEQ_LIST” created on Apr. 20, 2023 and having a size of 18,437 bytes. The contents of the Sequence Listing XML are incorporated by reference herein in their entirety.

### INTRODUCTION

Dopamine (DA) circuits play important roles in health (movement, reward, aversion, motivation) and disease (e.g., Parkinson's disease, schizophrenia, and addiction). DA neurons have diverse downstream targets, making it difficult to identify the casual basis of specific behavioral outcomes. For example, the striatum is a hub region that receives dense dopaminergic innervation from the midbrain. Multiple distinct, spatially intermixed and inter-connected striatal cell types express various combinations of four of the five DA receptor (DAR) subtypes, the G_{s/olf}-coupled D1-like receptors D1R and D5R and the G_{i/o/z}-coupled D2-like receptors D2R and D3R.

There is a need in the art for methods that can control specific DARs in neurons.

### SUMMARY

The present disclosure provides conjugates and systems for modulating the activity of a ligand-binding polypeptide such as a D2 dopamine receptor. The present disclosure provides methods of modulating the activity of a D2 dopamine receptor. The present disclosure provides methods of treating Parkinson's disease in an individual.

### BRIEF DESCRIPTION OF THE DRAWINGS

FIG. 1 presents a design of Photoswitchable Orthogonal Remotely Tethered Ligands (P) for D2R.

FIG. 2A-2F depict synthesis of P-D2ago and photophysical characterization.

FIG. 3A-3F depict photoactivation of D2R by MP-D2ago.

FIG. 4 depicts photoactivation of D2-like receptors, and lack of photoactivation of D1-like receptors, by MP-D2ago.

FIG. 5A-5G depict evaluation of phenylpiperazine-based MPs of D2R in HEK293T cells.

FIG. 6 depicts chemical Structures of D1-like receptor ligands based on the benzazepine scaffold, a conformationally restricted version of dopamine.

FIG. 7 depicts multistep chemical synthesis of P-D1block.

FIG. 8A-8E depict photophysical Characterization of P-D1block(24).

FIG. 9A-9H depict patch clamp electrophysiology of P-D1block in HEK293T cells.

### DEFINITIONS

The term “alkyl” refers to a monoradical branched or unbranched saturated hydrocarbon chain, e.g., having from 1 to 40 carbon atoms, from 1 to 10 carbon atoms, or from 1 to 6 carbon atoms. This term is exemplified by groups such as methyl, ethyl, n-propyl, iso-propyl, n-butyl, iso-butyl, n-hexyl, n-decyl, tetradecyl, and the like.

The term “substituted alkyl” refers to an alkyl group as defined above wherein one or more carbon atoms in the alkyl chain have been optionally replaced with a heteroatom such as —O—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}Rb, wherein R^{a} and R^{b} may be the same or different and are chosen from hydrogen, optionally substituted alkyl, cycloalkyl, alkenyl, cycloalkenyl, alkynyl, aryl, heteroaryl and heterocyclic.

The term “alkylaminoalkyl”, “alkylaminoalkenyl” and “alkylaminoalkynyl” refers to the groups R^{a}NHR^{b}— where R^{a} is alkyl group as defined above and R^{b} is alkylene, alkenylene or alkynylene group as defined above.

The term “alkaryl” or “aralkyl” refers to the groups -alkylene-aryl and -substituted alkylene-aryl where alkylene, substituted alkylene and aryl are defined herein.

The term “alkoxy” refers to the groups alkyl-O—, alkenyl-O—, cycloalkyl-O—, cycloalkenyl-O—, and alkynyl-O—, where alkyl, alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “haloalkoxy” refers to the groups alkyl-O— wherein one or more hydrogen atoms on the alkyl group have been substituted with a halo group and include, by way of examples, groups such as trifluoromethoxy, and the like.

The term “alkylalkoxy” refers to the groups -alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

The term “alkylthioalkoxy” refers to the group -alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein.

The term “alkenyl” refers to a monoradical of a branched or unbranched unsaturated hydrocarbon group having from 2 to 40 carbon atoms, from 2 to 10 carbon atoms, or from 2 to 6 carbon atoms and having at least 1 site (e.g., from 1-6 sites) of vinyl unsaturation.

The term “substituted alkenyl” refers to an alkenyl group as defined above having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “alkynyl” refers to a monoradical of an unsaturated hydrocarbon having from 2 to 40 carbon atoms, from 2 to 20 carbon atoms, or from 2 to 6 carbon atoms and having at least 1 site (e.g., from 1-6 sites) of acetylene (triple bond) unsaturation.

The term “substituted alkynyl” refers to an alkynyl group as defined above having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, and —SO₂-heteroaryl.

The term “acyl” refers to the groups HC(O)—, alkyl-C(O)—, substituted alkyl-C(O)—, cycloalkyl-C(O)—, substituted cycloalkyl-C(O)—, cycloalkenyl-C(O)—, substituted cycloalkenyl-C(O)—, aryl-C(O)—, heteroaryl-C(O)— and heterocyclic-C(O)— where alkyl, substituted alkyl, cycloalkyl, substituted cycloalkyl, cycloalkenyl, substituted cycloalkenyl, aryl, heteroaryl, and heterocyclic are as defined herein.

The term “acylamino” or “aminocarbonyl” refers to the group —C(O)NRR where each R is independently hydrogen, alkyl, substituted alkyl, aryl, heteroaryl, heterocyclic or where both R groups are joined to form a heterocyclic group (e.g., morpholino) wherein alkyl, substituted alkyl, aryl, heteroaryl, and heterocyclic are as defined herein.

The term “aminoacyl” refers to the group —NRC(O)R where each R is independently hydrogen, alkyl, substituted alkyl, aryl, heteroaryl, or heterocyclic wherein alkyl, substituted alkyl, aryl, heteroaryl, and heterocyclic are as defined herein.

The term “aminoacyloxy” or “alkoxycarbonylamino” refers to the group —NRC(O)OR where each R is independently hydrogen, alkyl, substituted alkyl, aryl, heteroaryl, or heterocyclic wherein alkyl, substituted alkyl, aryl, heteroaryl, and heterocyclic are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclic-C(O)O— wherein alkyl, substituted alkyl, cycloalkyl, substituted cycloalkyl, aryl, heteroaryl, and heterocyclic are as defined herein.

The term “aryl” refers to an unsaturated aromatic carbocyclic group of from 6 to 20 carbon atoms having a single ring (e.g., phenyl) or multiple condensed (fused) rings (e.g., naphthyl or anthryl). Exemplary aryls include phenyl, naphthyl and the like. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halo, nitro, heteroaryl, heteroaryloxy, heterocyclic, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

The term “aryloxy” refers to the group aryl-O— wherein the aryl group is as defined above including optionally substituted aryl groups as also defined herein.

The term “amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclic provided that both R's are not hydrogen.

The term “carboxyalkyl” or “alkoxycarbonyl” refers to the groups “—C(O)O-alkyl”, “—C(O)O-substituted alkyl”, “—C(O)O-cycloalkyl”, “—C(O)O-substituted cycloalkyl”, “—C(O)O-alkenyl”, “—C(O)O-substituted alkenyl”, “—C(O)O-alkynyl” and “—C(O)O-substituted alkynyl” where alkyl, substituted alkyl, cycloalkyl, substituted cycloalkyl, alkenyl, substituted alkenyl, alkynyl and substituted alkynyl alkynyl are as defined herein.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “cycloalkenyl” refers to cyclic alkenyl groups of from 4 to 20 carbon atoms having a single cyclic ring and at least one point of internal unsaturation. Examples of suitable cycloalkenyl groups include, for instance, cyclobut-2-enyl, cyclopent-3-enyl, cyclooct-3-enyl, and the like.

The term “substituted cycloalkenyl” refers to cycloalkenyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “halo” or “halogen” refers to fluoro, chloro, bromo and iodo.

The term “heteroaryl” refers to an aromatic group of from 1 to 15 carbon atoms and 1 to 4 heteroatoms selected from oxygen, nitrogen and sulfur within at least one ring (if there is more than one ring). Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halo, nitro, heteroaryl, heteroaryloxy, heterocyclic, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

The term “heteroaralkyl” refers to the groups -alkylene-heteroaryl where alkylene and heteroaryl are defined herein. Such heteroaralkyl groups are exemplified by pyridylmethyl, pyridylethyl, indolylmethyl, and the like.

The term “heteroaryloxy” refers to the group heteroaryl-O—.

The term “heterocycle” or “heterocyclic” refers to a monoradical saturated or unsaturated group having a single ring or multiple condensed rings, from 1 to 40 carbon atoms and from 1 to 10 hetero atoms, e.g., from 1 to 4 heteroatoms, selected from nitrogen, sulfur, phosphorus, and/or oxygen within the ring. Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

Examples of nitrogen heteroaryls and heterocycles include, but are not limited to, pyrrole, thiophene, furan, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, pyrrolidine, piperidine, piperazine, indoline, morpholine, tetrahydrofuranyl, tetrahydrothiophene, and the like as well as N-alkoxy-nitrogen containing heterocycles.

The term “heterocyclooxy” refers to the group heterocyclic-O—.

The term “heterocyclothio” refers to the group heterocyclic-S—.

The term “heterocyclene” refers to the diradical group formed from a heterocycle, as defined herein, and is exemplified by the groups 2,6-morpholino, 2,5-morpholino and the like.

The term “heteroarylamino” refers to a 5 membered aromatic ring wherein one or two ring atoms are N, the remaining ring atoms being C. The heteroarylamino ring may be fused to a cycloalkyl, aryl or heteroaryl ring, and it may be optionally substituted with one or more substituents, e.g., one or two substituents, selected from alkyl, substituted alkyl, cycloalkyl, aryl, aralkyl, heteroaryl, heteroaralkyl, halo, cyano, acyl, amino, substituted amino, acylamino, —OR (where R is hydrogen, alkyl, alkenyl, cycloalkyl, acyl, aryl, heteroaryl, aralkyl, or heteroaralkyl), or —S(O)ₙR where n is an integer from 0 to 2 and R is hydrogen (provided that n is 0), alkyl, alkenyl, cycloalkyl, amino, heterocyclo, aryl, heteroaryl, aralkyl, or heteroaralkyl.

The term “heterocycloamino” refers to a saturated monovalent cyclic group of 4 to 8 ring atoms, wherein at least one ring atom is N and optionally contains one or two additional ring heteroatoms selected from the group consisting of N, O, or S(O)n (where n is an integer from 0 to 2), the remaining ring atoms being C, where one or two C atoms may optionally be replaced by a carbonyl group. The heterocycloamino ring may be fused to a cycloalkyl, aryl or heteroaryl ring, and it may be optionally substituted with one or more substituents, e.g., one or two substituents, selected from alkyl, substituted alkyl, cycloalkyl, aryl, aralkyl, heteroaryl, heteroaralkyl, halo, cyano, acyl, amino, substituted amino, acylamino, —OR (where R is hydrogen, alkyl, alkenyl, cycloalkyl, acyl, aryl, heteroaryl, aralkyl, or heteroaralkyl), or —S(O)ₙR [where n is an integer from 0 to 2 and R is hydrogen (provided that n is 0), alkyl, alkenyl, cycloalkyl, amino, heterocyclo, aryl, heteroaryl, aralkyl, or heteroaralkyl].

The term “oxyacylamino” or “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

The term “thiol” refers to the group —SH.

The term “thioalkoxy” or “alkylthio” refers to the group —S-alkyl.

The term “substituted thioalkoxy” refers to the group —S-substituted alkyl.

The term “thioaryloxy” refers to the group aryl-S— wherein the aryl group is as defined above including optionally substituted aryl groups also defined above.

The term “thioheteroaryloxy” refers to the group heteroaryl-S— wherein the heteroaryl group is as defined above including optionally substituted aryl groups as also defined above.

As to any of the above groups which contain one or more substituents, it is understood, of course, that such groups do not contain any substitution or substitution patterns which are sterically impractical and/or synthetically non-feasible. In addition, the compounds of the embodiments include all stereochemical isomers arising from the substitution of these compounds.

The term “pharmaceutically-acceptable salt” refers to salts which retain biological effectiveness and are not biologically or otherwise undesirable. In many cases, the compounds of the embodiments are capable of forming acid and/or base salts by virtue of the presence of amino and/or carboxyl groups or groups similar thereto.

Pharmaceutically-acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

The terms “polypeptide,” “peptide,” and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component.

A polypeptide has a certain percent “sequence identity” to another polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wisconsin, USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, California, USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970)

Of interest is the BestFit program using the local homology algorithm of Smith Waterman (Advances in Applied Mathematics 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wisconsin, USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters:
Mismatch Penalty: 1.00;
Gap Penalty: 1.00;
Gap Size Penalty: 0.33; and
Joining Penalty: 30.0.

The term “linker” or “linkage” refers to a linking moiety that connects two groups and has a backbone of 100 atoms or less in length. A linker or linkage may be a covalent bond that connects two groups or a chain of between 1 and 100 atoms in length, for example a chain of 1, 2, 3, 4, 5, 6, 8, 10, 12, 14, 16, 18, 20 or more carbon atoms in length, where the linker may be linear, branched, cyclic or a single atom. In some cases, the linker is a branching linker that refers to a linking moiety that connects three or more groups. In certain cases, one, two, three, four or five or more carbon atoms of a linker backbone may be optionally substituted with a sulfur, nitrogen or oxygen heteroatom. In some cases, the linker backbone includes a linking functional group, such as an ether, thioether, amino, amide, sulfonamide, carbamate, thiocarbamate, urea, thiourea, ester, thioester or imine. The bonds between backbone atoms may be saturated or unsaturated, and in some cases not more than one, two, or three unsaturated bonds are present in a linker backbone. The linker may include one or more substituent groups, for example with an alkyl, aryl or alkenyl group. A linker may include, without limitations, polyethylene glycol; ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable.

The terms “polyethylene oxide”, “PEO”, “polyethylene glycol” and “PEG” are used interchangeably and refer to a polymeric group including a chain described by the formula —(CH₂—CH₂—O—)ₙ— or a derivative thereof. In some embodiments, “n” is 5000 or less, such as 1000 or less, 500 or less, 200 or less, 100 or less, 50 or less, 40 or less, 30 or less, 20 or less, 15 or less, such as 3 to 15, or 10 to 15.

The terms “antibodies” and “immunoglobulin” include antibodies or immunoglobulins of any isotype, fragments of antibodies that retain specific binding to antigen (e.g., to a target ligand-binding polypeptide), including, but not limited to, Fab, Fv, scFv, and Fd fragments, chimeric antibodies, humanized antibodies, single-chain antibodies (scAb), single domain antibodies (sdAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. Also encompassed by the term are Fab′, Fv, F(ab′)₂, and or other antibody fragments that retain specific binding to antigen, and monoclonal antibodies.

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids. In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

Cartilaginous fishes also have heavy-chain antibodies (IgNAR; “immunoglobulin new antigen receptor”), from which single-domain antibodies called V_{NAR} fragments can be obtained. Thus, in some cases, an affinity agent is an IgNAR.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al. (1995) Protein Eng. 8(10): 1057-1062); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen. Antibody fragments include, e.g., scFv, sdAb, dAb, Fab, Fab′, Fab′₂, F(ab′)₂, Fd, Fv, Feb, and SMIP. Examples of sdAb are a camelid VHH and a cartilaginous fish VNAR.

“Fv” is the minimum antibody fragment that contains a complete antigen-recognition and -binding site. This region consists of a dimer of one heavy- and one light-chain variable domain in tight, non-covalent association. It is in this configuration that the three complementarity determining regions (CDRs) of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease or at risk of acquiring the disease but has not yet been diagnosed as having it; (b) inhibiting the disease, i.e., arresting its development; (c) relieving the disease, i.e., causing regression of the disease; and (d) replacing a lost function that results from the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), lagomorphs, etc. In some cases, the individual is a human. In some cases, the individual is a non-human primate. In some cases, the individual is a rodent, e.g., a rat or a mouse. In some cases, the individual is a lagomorph, e.g., a rabbit.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a ligand” includes a plurality of such ligands and reference to “the D2 dopamine receptor agonist” includes reference to one or more D2 dopamine receptor agonists and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides conjugates and systems for modulating the activity of a ligand-binding polypeptide such as a D2 dopamine receptor. The present disclosure provides methods of modulating the activity of a D2 dopamine receptor. The present disclosure provides methods of treating Parkinson's disease in an individual.

### Systems

The present disclosure provides systems for modulating a D2 dopamine receptor in an individual. A system of the present disclosure includes: a) a conjugate comprising: i) an affinity agent that forms a covalent bond with a self-labeling protein tag; ii) a linker; iii) a photoisomerizable group; and iv) a ligand that binds to a target ligand-binding polypeptide. A system of the present disclosure also includes either: b1) a fusion polypeptide, or a recombinant expression vector comprising a nucleotide sequence encoding the fusion polypeptide, wherein the fusion polypeptide comprises: i) a self-labeling protein tag; ii) a peptide linker; and iii) a membrane-anchoring polypeptide; or b2) a fusion polypeptide, or a recombinant expression vector comprising a nucleotide sequence encoding the fusion polypeptide, wherein the fusion polypeptide comprises: i) a self-labeling protein tag; and ii) an antibody specific for a D2 dopamine receptor.

### Conjugates

A conjugate suitable for inclusion in a system of the present disclosure includes a compound as described below.

In certain embodiments, compounds of interest include a compound of Formula I:

DRL-HP-L-HT (I)

wherein:
DRL is a dopamine receptor ligand;
HP is a hydrophobic moiety;
L is a linker; and
H is a halogen-containing substrate.

In some cases, the DRL is a D2 dopamine receptor agonist. In some cases, the DRL is a D2 dopamine receptor antagonist. In some cases, the DRL is a D1 dopamine receptor antagonist.

In some embodiments, the dopamine receptor ligand (DRL) comprises an aminoindenyl moiety. In some instances, the dopamine receptor ligand comprises a N-propyl 2-aminoindenyl moiety. In some embodiments, the dopamine receptor ligand comprises a phenylpiperazine moiety. In some instances, the dopamine receptor ligand comprises a halo-substituted phenylpiperazine moiety. In some instances, the dopamine receptor ligand comprises an alkoxy substituted phenylpiperazine moiety, such as a methoxy substituted phenylpiperazine moiety. In some embodiments, the hydrophobic moiety (HP) comprises an azobenzene moiety. In some embodiments, the linker comprises a polyalkylene glycol, such as a polyethylene glycol where the polyalkylene glycol has n polymer units of 8 or more, such as 9, 10, 11, 12, 13, 14, 15, 16, 17, 18, 19, 20, 21, 22, 23 and including where the polyalkylene glycol has n polymer units of 24 or more. In certain instances, the linker comprises a polyethylene glycol having 24 polymer units. In some embodiments, the linker comprises a heterobifunctional component, such as a bis-amide component. In certain embodiments, the linker comprises a heterobifunctional terminated polyalkylene glycol. In some instances, the linker comprises a heterobifunctional, carboxylic acid-terminated polyethylene glycol, such as a heterobifunctional, carboxylic acid-terminated PEG[24]. In some embodiments, the halogen-containing substrate is a haloalkane substrate, such as a chloroalkane substrate.

In some embodiments, compounds of interest include a compound of Formula IA:

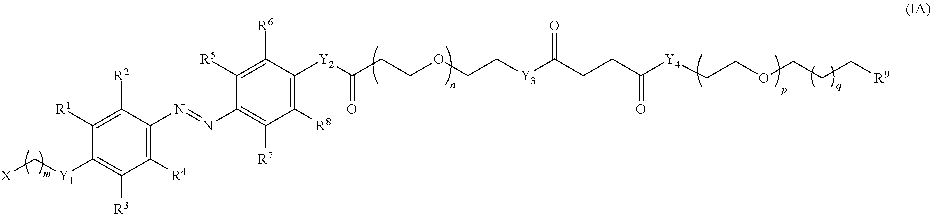

wherein X is:

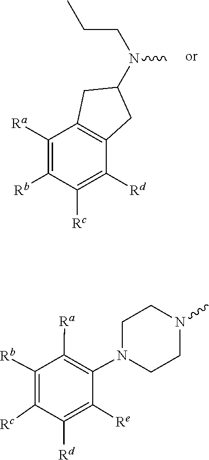

wherein [symbol] represents the X—(C)ₘ— bond;
m is an integer from 1-10;
n is an integer from 1-50;
p is an integer from 1-10;
q is an integer from 1-25;
Y₁, Y₂, Y₃ and Y₄ are independently selected from C, N, O or S;
R^{a}, R^{b}, R^{c} and R^{d} are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
R¹, R², R³, R⁴, R⁵, R⁶, R⁷ and R⁸ are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
R⁹ is selected from hydrogen, halogen, alkoxy, nitro, amino, hydroxy, cyano, thiol.

In some embodiments, one or more of R^{a}, R^{b}, R^{c} and R^{d} is independently selected from F, Cl, Br and I. In certain instances, one or more of R^{a}, R^{b}, R^{c} and R^{d} is Cl. In certain embodiments, R^{a} and R^{b} are Cl. In some embodiments, one or more of R^{a}, R^{b}, R^{c} and R^{d} is a C(1-6) alkoxy, such as methoxy or ethoxy. In certain instances, R^{a} is methoxy.

In some embodiments, R¹, R², R³, R⁴, R⁵, R⁶, R⁷ and R⁸ are hydrogen. In some embodiments, one or more of R¹, R², R³, R⁴, R⁵, R⁶, R⁷ and R⁸ is a C(1-8) alkyl, such as a C(1-8) linear alkyl or C(1-8) branched alkyl. For example, one or more of R¹, R², R³, R⁴, R⁵, R⁶, R⁷ and R⁸ may be methyl, ethyl, n-propyl, isopropyl, n-butyl, isobutyl, pentyl, isopentyl, hexyl, heptyl, octyl.

In certain embodiments, m is an integer from 2-8, such as from 2-6. In certain instances, m is 4. In certain instances, m is 3.

In certain embodiments, n is an integer from 12-30, such as from 16-28. In certain instances, n is 24.

In certain embodiments, p is an integer from 2-8, such as from 2-6. In certain instances, p is 2.

In certain embodiments, q is an integer from 2-12, such as from 2-8. In certain instances, q is 4.

In certain embodiments, R⁹ is selected from F, Cl, Br and I. In certain instances, R⁹ is Cl.

In certain embodiments, a conjugate is a compound of Formula 1A:

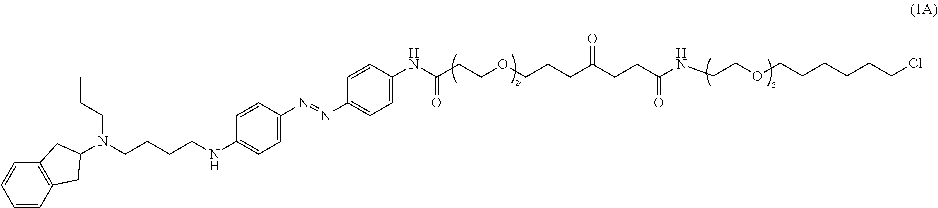

In certain embodiments, a conjugate is a compound of Formula 1B:

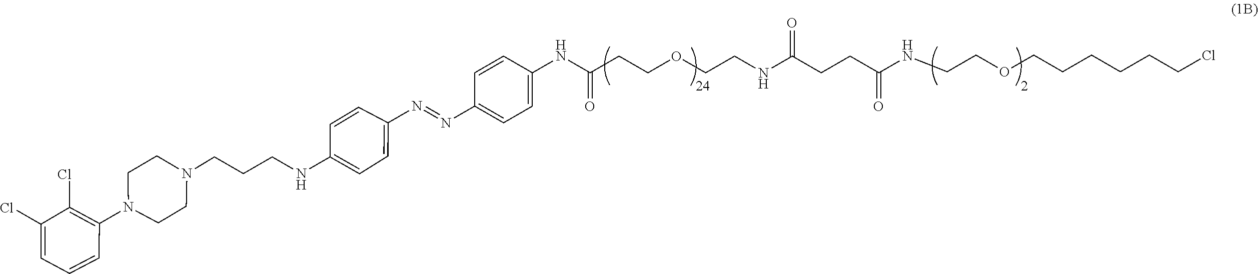

In certain embodiments, a conjugate is a compound of Formula 1C:

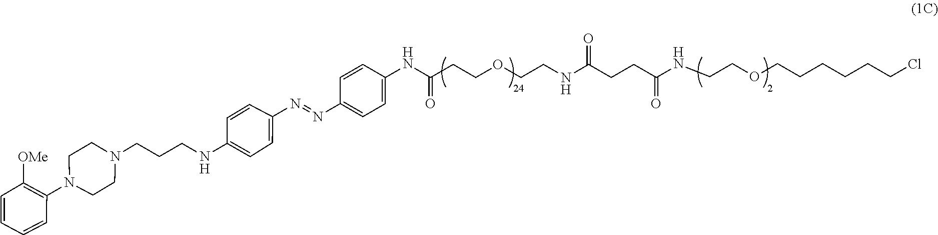

In some embodiments, compounds of interest include a compound of Formula IIA:

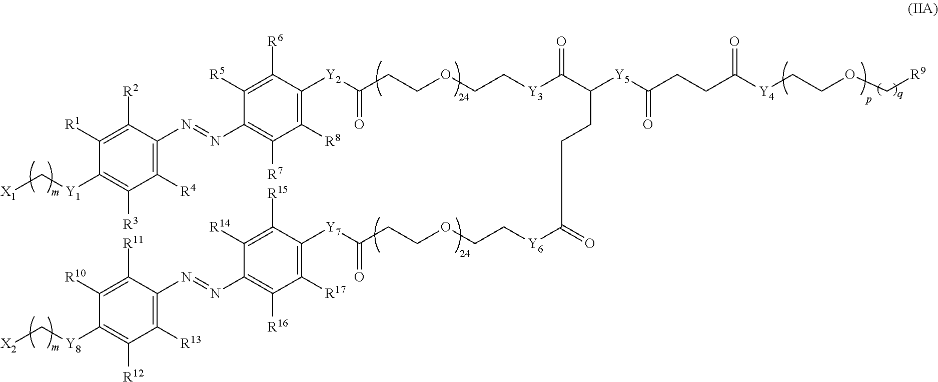

wherein X₁ and X₂ are independently selected from:

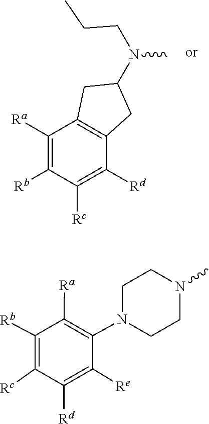

wherein [symbol] represents the X—(C)ₘ— bond;
m is an integer from 1-10;
n is an integer from 1-50;
p is an integer from 1-10;
q is an integer from 1-25;
Y₁, Y₂, Y₃, Y₄, Y₅, Y₆, Y₇ and Y₈ are independently selected from C, N, O or S;
R^{a}, R^{b}, R^{c} and R^{d} are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
R¹, R², R³, R⁴, R⁵, R⁶, R⁷, R⁸, R¹⁰, R¹¹, R¹², R¹³, R¹⁴, R¹⁵, R¹⁶ and R¹⁷ are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
R⁹ is selected from hydrogen, halogen, alkoxy, nitro, amino, hydroxy, cyano, thiol.

In some embodiments, one or more of R^{a}, R^{b}, R^{c} and R^{d} is independently selected from F, Cl, Br and I. In certain instances, one or more of R^{a}, R^{b}, R^{c} and R^{d} is Cl. In certain embodiments, R^{a} and R^{b} are Cl. In some embodiments, one or more of R^{a}, R^{b}, R^{c} and R^{d} is a C(1-6) alkoxy, such as methoxy or ethoxy. In certain instances, R^{a} is methoxy.

In some embodiments, R¹, R², R³, R⁴, R⁵, R⁶, R⁷, R⁸, R¹⁰, R¹¹, R¹², R¹³, R¹⁴, R¹⁵, R¹⁶ and R¹⁷ are hydrogen. In some embodiments, one or more of R¹, R², R³, R⁴, R⁵, R⁶, R⁷, R⁸, R¹⁰, R¹¹, R¹², R¹³, R¹⁴, R¹⁵, R¹⁶ and R¹⁷ are is a C(1-8) alkyl, such as a C(1-8) linear alkyl or C(1-8) branched alkyl. For example, one or more of R¹, R², R³, R⁴, R⁵, R⁶, R⁷ and R⁸ may be methyl, ethyl, n-propyl, isopropyl, n-butyl, isobutyl, pentyl, isopentyl, hexyl, heptyl, octyl.

In certain embodiments, m is an integer from 2-8, such as from 2-6. In certain instances, m is 4. In certain instances, m is 3.

In certain embodiments, n is an integer from 12-30, such as from 16-28. In certain instances, n is 24.

In certain embodiments, p is an integer from 2-8, such as from 2-6. In certain instances, p is 2.

In certain embodiments, q is an integer from 2-12, such as from 2-8. In certain instances, q is 4.

In certain embodiments, R⁹ is selected from F, Cl, Br and I. In certain instances, R⁹ is Cl.

In some embodiments, compounds of interest include a compound of Formula 2A:

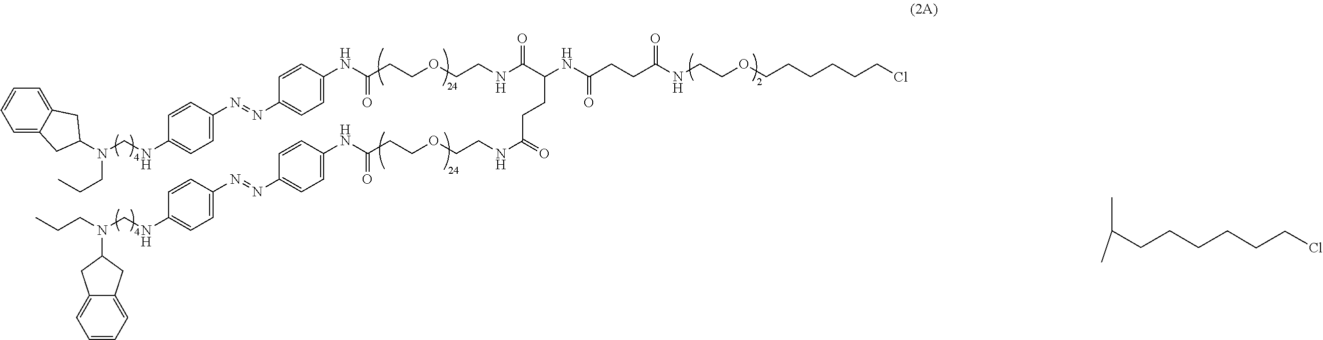

In some cases, the DRL is a D2 receptor antagonist. For example, in some cases, a conjugate has the following structure:

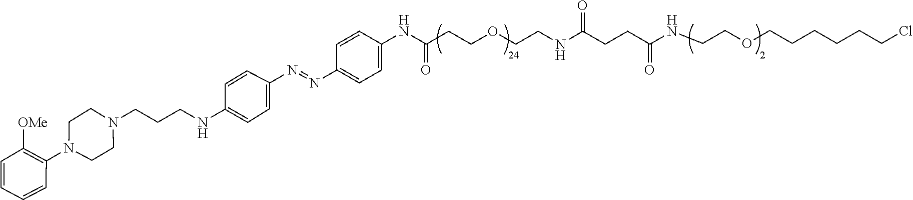

As another example, in some cases, a conjugate has the following structure:

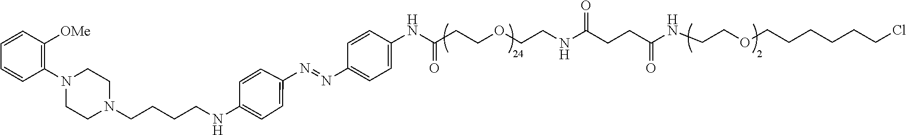

In some cases, the DRL is a D1 receptor antagonist

For example, in some cases, a conjugate has the following structure:

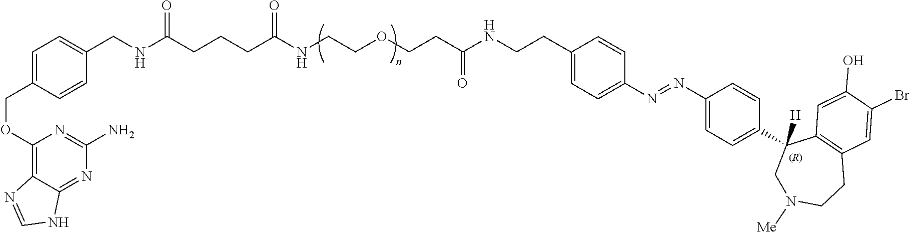

### Affinity Agents (“H”)

Halogen-containing substrates (“H”), also referred to as “affinity agents”, suitable for inclusion in a conjugate include agents that bind to self-labeling polypeptides. Suitable affinity agents include nucleoside base derivatives. In some cases, the nucleoside base of the nucleoside base derivative is selected from guanine, cytosine, uracil, thymine, xanthine, and hypoxanthine. For example, the nucleoside base of the nucleoside base derivative can be guanine, xanthine or hypoxanthine. In some cases, the nucleoside base of the nucleoside base derivative is guanine. In other instances, the nucleoside base of the nucleoside base derivative can be cytosine, thymine or uracil. In some cases, the nucleoside base of the nucleoside base derivative is cytosine. The nucleoside base can be derivatized to provide the nucleoside base derivative of the affinity agent of a conjugate of the present disclosure. In some cases, the nucleoside base derivative of the affinity agent is a benzylnucleoside base, such as benzylguanine or benzoylcytosine. In some cases, the affinity agent is benzylguanine. In embodiments where the affinity agent is benzylguanine, the benzylguanine affinity agent may provide for covalent binding to a SNAP tag. In some cases, the affinity agent is benzoylcytosine. In embodiments where the affinity agent is benzoylcytosine, the benzoylcytosine affinity agent may provide for covalent binding to a CLIP tag. In some cases, the affinity agent is a chloropyrimidine; a chloropyrimidine can bind to a SNAP tag.

Suitable affinity agents also include alkyl derivatives, such as haloalkyl derivatives where one or more hydrogen atoms in an alkyl or alkyl derivative is replaced by a halogen, e.g., fluoro, chloro, or bromo. In some cases, the haloalkyl derivative is a fluoroalkane. In some cases, the haloalkyl derivative is a chloroalkane. In some cases, the haloalkyl derivative is a bromoalkane. In some cases, the affinity agent is chloroalkane, such as Cl(CH₂)₆(OCH₂CH₂)₂. In embodiments where the affinity agent is chloroalkane, the chloroalkane affinity agent may provide for covalent binding to a HALO tag.

### Photoisomerizable Groups (“HP”)

Hydrophobic moieties (HP; also referred to herein as “photoisomerizable groups”) are known in the art, and any known photoisomerizable group can be included in a conjugate of the present disclosure. Suitable photoisomerizable groups include, but are not limited to, azobenzene, cyclic azobenzenes and azoheteroarenes and derivatives thereof; spiropyran and derivatives thereof; triphenyl methane and derivatives thereof; 4,5-epoxy-2-cyclopentene and derivatives thereof; fulgide and derivatives thereof; thioindigo and derivatives thereof; diarylethene and derivatives thereof; diallylethene and derivatives thereof; overcrowded alkenes and derivatives thereof; and anthracene and derivatives thereof. In some cases, a suitable photoisomerizable group is a photoisomerizable group as shown in the examples herein.

Suitable spiropyran derivatives include, but are not limited to, 1,3,3-trimethylindolinobenzopyrylospiran; 1,3,3-trimethylindolino-6′-nitrobenzopyrylospiran; 1,3,3-trimethylindolino-6′-bromobenzopyrylospiran; 1-n-decyl-3,3-dimethylindolino-6′-nitrobenzopyrylospiran; 1-n-octadecy-1-3,3-dimethylindolino-6′-nitrobenzopyrylospiran; 3′,3′-dimethyl-6-nitro-1′-[2-(phenylcarbamoyl)ethyl]spiro; [2H-1-benzopyran-2,2′-indoline]; 1,3,3-trimethylindolino-8′-methoxybenzopyrylospiran; and 1,3,3-trimethylindolino-β-naphthopyrylospiran. Also suitable for use is a merocyanine form corresponding to spiropyran or a spiropyran derivative.

Suitable triphenylmethane derivatives include, but are not limited to, malachite green derivatives. specifically, there can be mentioned, for example, bis[dimethylamino)phenyl] phenylmethanol, bis[4-(diethylamino)phenyl]phenylmethanol, bis[4-(dibutylamino)phenyl]phenylmethanol and bis[4-(diethylamino)phenyl]phenylmethane.

Suitable 4,5-epoxy-2-cyclopentene derivatives include, for example, 2,3-diphenyl-1-indenone oxide and 2′,3′-dimethyl-2,3-diphenyl-1-indenone oxide.

Suitable azobenzene compounds include, e.g., compounds having azobenzene residues crosslinked to a side chain, e.g., compounds in which 4-carboxyazobenzene is ester bonded to the hydroxyl group of polyvinyl alcohol or 4-carboxyazobenzene is amide bonded to the amino group of polyallylamine. Also suitable are azobenzene compounds having azobenzene residues in the main chain, for example, those formed by ester bonding bis(4-hydroxyphenyl)dimethylmethane (also referred to as bisphenol A) and 4,4′-dicarboxyazobenzene or by ester bonding ethylene glycol and 4,4′-dicarboxyazobenzene.

Suitable cyclic azobenzene and azoheteroarene compounds which can be adapted for use in the subject conjugates and photoisomerizable regulators include, but are not limited to, 11,12-dihydrodibenzo[c,g][1,2]diazocine-5-oxide,

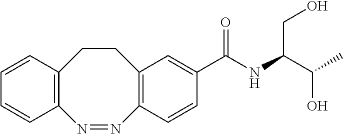 [SMILES: C[C@H](O)[C@H](CO)NC(=O)c1ccc2c(c1)CCc1ccccc1/N=N\2]

heterodiazocines, such as those photoswitches described by Hammerich et al. J. Am. Chem. Soc., 2016, 138 (40), pp 13111-13114), and azoheteroarene photoswitches such as 3-pyrazoles (3pzH or 3pzMe), 5-pyrazoles (5pzH or 5pzMe), 3-pyrroles (3pyH or 3pyMe), triazole and tetrazoles (tet or 3tri) as describes by Calbo et al. J. Am. Chem. Soc., 2017, 139 (3), pp 1261-1274, the disclosure of which is herein incorporated by reference.

Suitable fulgide derivatives include, but are not limited to, isopropylidene fulgide and adamantylidene fulgide.

Suitable diallylethene derivatives include, for example, 1,2-dicyano-1,2-bis(2,3,5-trimethyl-4-thienyl)ethane; 2,3-bis(2,3,5-trimethyl-4-thiethyl) maleic anhydride; 1,2-dicyano-1,2-bis(2,3,5-trimethyl-4-selenyl)ethane; 2,3-bis(2,3,5-trimethyl-4-selenyl) maleic anhydride; and 1,2-dicyano-1,2-bis(2-methyl-3-N-methylindole)ethane.

Suitable diarylethene derivatives include but are not limited to, substituted perfluorocylopentene-bis-3-thienyls and bis-3-thienylmaleimides.

Suitable overcrowded alkenes include, but are not limited to, cis-2-nitro-7-(dimethylamino)-9-(2′,3′-dihydro-1′H-naphtho[2,1-b]thiopyran-1′-ylidene)-9H-thioxanthene and trans-dimethyl-[1-(2-nitro-thioxanthen-9-ylidene)-2,3-dihydro-1H-benzo[f]thiochromen-8-yl]amine. Overcrowded alkenes are described in the literature. See, e.g., terWiel et al. (2005) Org. Biomol. Chem. 3:28-30; and Geertsema et al. (1999) Agnew CHem. Int. Ed. Engl. 38:2738.

Other suitable photoisomerizable groups include, e.g., reactive groups commonly used in affinity labeling, including diazoketones, aryl azides, diazerenes, and benzophenones.

In some instances, the photoisomerizable group of the conjugate (e.g., as defined herein) is an azobenzene (e.g., an azobenzene photoswitch) of the following formula:

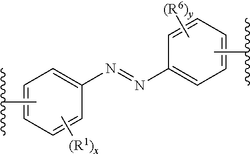

wherein:
R¹ and R⁶ are one or more optional substituents selected from hydrogen, C₁₋₁₀ alkyl, substituted C₁₋₁₀ alkyl, —NR¹⁰R¹¹, —NR¹²C(O)R¹³, —NR¹²C(O)OR¹³, —NR¹²C(O)NR¹²R¹³, C₂₋₁₀ alkenyl, substituted C_{2 10} alkenyl, C_{2 10} alkynyl, substituted C_{2 10} alkynyl, C_{6 20} aryl, substituted C_{6 20} aryl, heteroaryl, heterocyclic, heterocyclooxy, heterocyclothio, heteroarylamino, heterocycloamino, C₄₋₁₀ cycloalkyl, substituted C4-10 cycloalkyl, C₄₋₁₀ cycloalkenyl, substituted C₄₋₁₀ cycloalkenyl, cyano, halo, —OR¹⁰, —C(O)OR¹⁰, —SR¹⁰, —S(O)R¹⁰, —S(O)₂R¹⁰;
x is an integer from 1 to 5;
y is an integer from 1 to 5; and
   wherein R¹⁰-R¹³ are as defined below,
   or a pharmaceutically acceptable salt thereof.

In some cases, a photoisomerizable group present in a conjugate of the present disclosure is a compound of Formula 1:

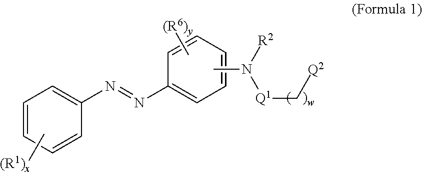

wherein:
Q¹ is —CH₂— or —C(═O)—;
Q² is a ligand (or a label or reactive group or second affinity agent), as described according to the present disclosure;
each R¹ is independently selected from hydrogen, C₁₋₁₀ alkyl, substituted C₁₋₁₀ alkyl, —NR¹⁰R¹¹, —NR¹²C(O)R¹³, —NR¹²C(O)OR¹³, —NR¹²C(O)NR¹²R¹³, C₂₋₁₀ alkenyl, substituted C₂₋₁₀ alkenyl, C_{2 10} alkynyl, substituted C_{2 10} alkynyl, C_{6 20} aryl, substituted C_{6 20} aryl, heteroaryl, heterocyclic, heterocyclooxy, heterocyclothio, heteroarylamino, heterocycloamino, C₄₋₁₀ cycloalkyl, substituted C4-10 cycloalkyl, C₄₋₁₀ cycloalkenyl, substituted C₄₋₁₀ cycloalkenyl, cyano, halo, —OR¹⁰, —C(O)OR¹⁰, —SR¹⁰, —S(O)R¹⁰, —S(O)₂R¹⁰;
w is an integer from 1 to 10;
x is an integer from 1 to 5;
y is an integer from 1 to 4;
R² is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl;
each R⁶ is independently selected from hydrogen, C₁₋₁₀ alkyl, substituted C₁₋₁₀ alkyl, —NR¹⁰R¹¹, —NR¹²C(O)R¹³, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, heteroaryl, heterocyclic, heterocyclooxy, heterocyclothio, heteroarylamino, heterocycloamino, C₄₋₁₀ cycloalkyl, substituted C₄₋₁₀ cycloalkyl, C₄₋₁₀ cycloalkenyl, substituted C₄₋₁₀ cycloalkenyl, cyano, halo, —OR¹⁰, —C(O)OR¹⁰, —SR¹⁰, —S(O)R¹⁰, —S(O)₂R¹⁰;
R¹⁰ and R¹¹ are each independently selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl;
R¹² is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl; and
R¹³ is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆-C₁₀ aryl, substituted C₆₋₂₀ aryl, C₄₋₁₀ cycloalkyl, substituted C₄₋₁₀ cycloalkyl, C₄₋₁₀ cycloalkenyl, substituted C₄₋₁₀ cycloalkenyl, —CH₂—N(CH₂CH₃)₃⁺, and —CH₂—SO₃⁻;
   or a pharmaceutically acceptable salt thereof.

In certain embodiments of Formula 1, Q¹ is —CH₂—. In certain embodiments of Formula 1, Q¹ is —C(═O)—.

In some instances of Formula 1, one of R¹ is linked via a linker to an affinity agent (e.g., as described herein). In some cases, the linker includes a branched linker (e.g., as described herein).

In some cases, a photoisomerizable group present in a conjugate of the present disclosure is a compound of Formula 2:

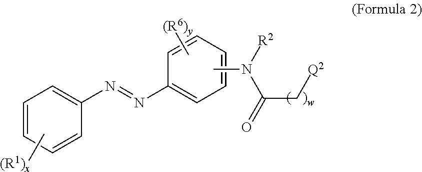

wherein
Q² is a ligand (or a label or reactive group or second affinity agent), as described according to the present disclosure;
each R¹ is independently selected from hydrogen, C₁₋₁₀ alkyl, substituted C₁₋₁₀ alkyl, —NR¹⁰R¹¹, —NR¹²C(O)R¹³, —NR¹²C(O)OR¹³, —NR¹²C(O)NR¹²R¹³, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, heteroaryl, heterocyclic, heterocyclooxy, heterocyclothio, heteroarylamino, heterocycloamino, C₄₋₁₀ cycloalkyl, substituted C₄₋₁₀ cycloalkyl, C₄₋₁₀ cycloalkenyl, substituted C₄₋₁₀ cycloalkenyl, cyano, halo, —OR¹⁰, —C(O)OR¹⁰, —SR¹⁰, —S(O)R¹⁰, —S(O)₂R₁₀;
w is an integer from 1 to 10;
x is an integer from 1 to 5;
y is an integer from 1 to 4;
R² is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl;
each R⁶ is independently selected from hydrogen, C₁₋₁₀ alkyl, substituted C₁₋₁₀ alkyl, —NR¹⁰R11, —NR¹²C(O)R¹³, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, heteroaryl, heterocyclic, heterocyclooxy, heterocyclothio, heteroarylamino, heterocycloamino, C₄₋₁₀ cycloalkyl, substituted C₄₋₁₀ cycloalkyl, C₄₋₁₀ cycloalkenyl, substituted C₄₋₁₀ cycloalkenyl, cyano, halo, —OR¹⁰, —C(O)OR¹⁰, —SR¹⁰, —S(O)R¹⁰, —S(O)₂R¹⁰;
R¹⁰ and R¹¹ are each independently selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl;
R¹² is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C6-20 aryl, substituted C6-20 aryl, C₄₋₁₀ cycloalkyl, substituted C₄₋₁₀ cycloalkyl, C₄₋₁₀ cycloalkenyl, and substituted C₄₋₁₀ cycloalkenyl; and
R¹³ is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆-C₁₀ aryl, substituted C₆₋₂₀ aryl, C₄₋₁₀ cycloalkyl, substituted C₄₋₁₀ cycloalkyl, C₄₋₁₀ cycloalkenyl, substituted C₄₋₁₀ cycloalkenyl, —CH₂—N(CH₂CH₃)³⁺, and —CH₂—SO₃⁻;
   or a pharmaceutically acceptable salt thereof.

In some instances of Formula 2, one of the R¹ groups is linked via a linker to an affinity agent (e.g., as described herein). In some cases, the linker includes a branched linker (e.g., as described herein).

In some cases, a photoisomerizable group present in a conjugate of the present disclosure is a compound of Formula 3:

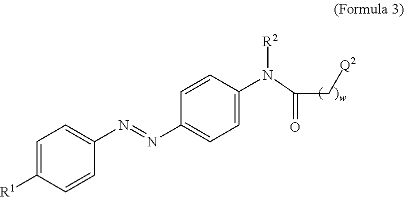

wherein:
Q² is a ligand (or a label or reactive group or second affinity agent), as described according to the present disclosure;
w is an integer from 1 to 10;
R¹ is selected from hydrogen, C₁₋₁₀ alkyl, —NR¹⁰R¹¹, —NR¹²C(O)R¹³, —NR¹²C(O)OR¹³ and —NR¹²C(O)NR¹²R¹³;
   R² is hydrogen or C₁₋₁₀ alkyl;
   R¹⁰ and R¹¹ are independently selected from hydrogen and C₁₋₁₀ alkyl;
   R¹² is hydrogen or C₁₋₁₀ alkyl; and
   R¹³ is selected from hydrogen, C₁₋₁₀ alkyl, C₁₋₈ alkenyl, C₆₋₁₀ aryl, and substituted C₁₋₁₀ alkyl,
   or a pharmaceutically acceptable salt thereof.

In certain embodiments of Formula 3, R¹ is C₁₋₁₀ alkyl, such as C₁₋₈ alkyl, e.g., C₁₋₆ alkyl, C₁₋₅ alkyl or C₁₋₄ alkyl. In some embodiments of Formula 3, R¹ is C₁₋₄ alkyl.

In certain embodiments of Formula 3, R¹ is —NR¹⁰R¹¹.

In certain embodiments of Formula 3, R¹ is —NR¹²C(O)R¹³.

In certain embodiment, R² is H.

In some instances of Formula 3, the R¹ group is linked via a linker to an affinity agent (e.g., as described herein). In some cases, the linker includes a branched linker (e.g., as described herein). For example, in embodiments where R¹ is —NR¹⁰R¹¹, the R¹ group can be linked via a linker to an affinity agent through either the R¹⁰ group or the R¹¹ group. In other cases, where R¹ is —NR¹²C(O)R¹³, the R¹ group can be linked via a linker to an affinity agent through the R¹³ group.

In some instances of Formulae 1, 2 or 3, Q² is a ligand, as described according to the present disclosure.

In some instances of Formulae 1, 2 or 3, Q² is a label, as described according to the present disclosure. For example, the label can be a detectable label, such as a fluorophore, as described herein.

In some instances of Formulae 1, 2 or 3, Q² is a reactive group, as described according to the present disclosure.

In some instances of Formulae 1, 2 or 3, Q² is a second affinity agent, as described according to the present disclosure.

In some cases, a photoisomerizable group present in a conjugate of the present disclosure is an azobenzene compound as shown below:

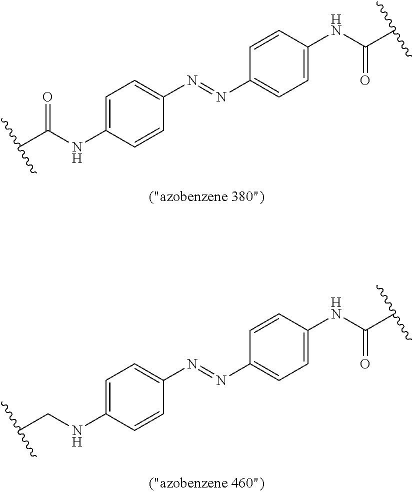 [SMILES: *C(=O)Nc1ccc(/N=N/c2ccc(NC(*)=O)cc2)cc1 ; *CNc1ccc(/N=N/c2ccc(NC(*)=O)cc2)cc1]

where the wavy lines indicate the attachment points to the rest of the conjugate. For instance, the wavy line on the left side of the azobenzene may indicate the attachment point to a linker (e.g., a branched linker as described herein) and the wavy line on the right side of the azobenzene may indicate the attachment point to a ligand as described herein.

In some cases, the photoisomerizable group is an azobenzene, such as an azobenzene photoisomerizable groups are found in WO 2019/060785, the disclosure of which is incorporated herein by reference in its entirety.

### Ligands (“DRL”)

As used herein, the term “ligand” or “DRL” refers to a molecule that binds to a polypeptide and effects a change in an activity of the polypeptide, and/or effects a change in conformation of the polypeptide, and/or affects binding of another polypeptide to the polypeptide, or affects the impact of another ligand on the polypeptide. Ligands include agonists, partial agonists, inverse agonists, antagonists, allosteric modulators, and blockers.

In some cases, a ligand is a small molecule ligand. Small molecule ligands can have a molecular weight in a range of from about 50 daltons to about 3000 daltons, e.g., from about 50 daltons to about 75 daltons, from about 75 daltons to about 100 daltons, from about 100 daltons to about 250 daltons, from about 250 daltons to about 500 daltons, from about 500 daltons to about 750 daltons, from about 750 daltons to about 1000 daltons, from about 1000 daltons to about 1250 daltons, from about 1250 daltons to about 1500 daltons, from about 1500 daltons to about 2000 daltons, from about 2000 daltons to about 2500 daltons, or from about 2500 daltons to about 3000 daltons.

In some cases, e.g., where the target ligand-binding polypeptide is a D2 dopamine receptor, the ligand is a D2 dopamine receptor agonist.

Dopamine has the following structure:

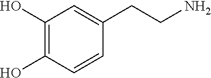 [SMILES: NCCc1ccc(O)c(O)c1]

Dihydrexidine has the following structure:

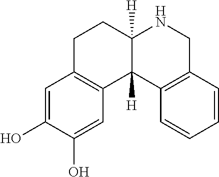 [SMILES: Oc1cc2c(cc1O)[C@H]1c3ccccc3CN[C@@H]1CC2]

In some cases, e.g., where the target ligand-binding polypeptide is a D2 dopamine receptor, the ligand is a D2 dopamine receptor antagonist, such as eticlopride; sulpiride; raclopride; L-741,626; domperidone; or an antipsychotic such as haloperidol, chlorpromazine, or clozapine.

In some cases, e.g., where the target ligand-binding polypeptide is a D2 dopamine receptor, the ligand is a D2 dopamine receptor-biased ligand. Examples of such ligands include dihydrexidine, aripiprazole, UNC9994, MLS1547. See, e.g., Agren et al. (2018) Int'l J. Neuropsychopharmacol. 21:1102.

UNC9994 has the following structure:

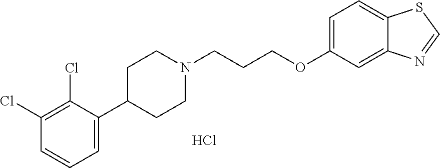 [SMILES: Clc1cccc(C2CCN(CCCOc3ccc4scnc4c3)CC2)c1Cl ; Cl]

MLS1547 has the following structure:

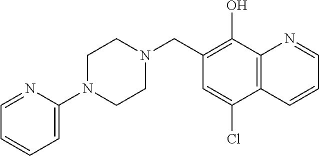 [SMILES: Oc1c(CN2CCN(c3ccccn3)CC2)cc(Cl)c2cccnc12]

In some cases, a dopamine receptor ligand (e.g., a D2 dopamine receptor ligand) comprises an aminoindenyl moiety. In some instances, the dopamine receptor ligand comprises a N-propyl 2-aminoindenyl moiety. In some cases, the dopamine receptor ligand comprises a phenylpiperazine moiety. In some instances, the dopamine receptor ligand comprises a halo-substituted phenylpiperazine moiety. In some instances, the dopamine receptor ligand comprises an alkoxy substituted phenylpiperazine moiety, such as a methoxy substituted phenylpiperazine moiety.

### Linkers

Suitable linkers include, but are not limited to, a polycarbon chain; poly(ethylene glycol); a peptide; and the like. In some cases, the linker is a C₁-C₂₅ alkyl. In some cases, the linker is a substituted C₁-C₂₅ alkyl. In some cases, the linker is poly(ethylene glycol) (PEG), where the PEG comprises from 2 to 50 ethylene glycol monomers; e.g., the PEG comprises from 2 to 5, from 5 to 10, from 10 to 15, from 15 to 20, from 20 to 25, from 25 to 30, from 30 to 35, from 35 to 40, from 40 to 45, or from 45 to 50, ethylene glycol units.

In some cases, the linker is a peptide of from 2 amino acids to 50 amino acids; e.g., from 2 amino acids to 5 amino acids, from 5 amino acids to 10 amino acids, from 10 amino acids to 15 amino acids, from 15 amino acids to 20 amino acids, from 20 amino acids to 25 amino acids, from 25 amino acids to 30 amino acids, or from 30 amino acids to 50 amino acids. In some cases, the linker is a peptide of 2, 3, 4, 5, 6, 7, 8, 9, or 10 amino acids in length.

### Fusion Polypeptides

As noted above, a system of the present disclosure includes, in addition to a conjugate as described above, either: b1) a fusion polypeptide, or a recombinant expression vector comprising a nucleotide sequence encoding the fusion polypeptide, wherein the fusion polypeptide comprises: i) a self-labeling protein tag; ii) a peptide linker; and iii) a membrane-anchoring polypeptide; or b2) a fusion polypeptide, or a recombinant expression vector comprising a nucleotide sequence encoding the fusion polypeptide, wherein the fusion polypeptide comprises: i) a self-labeling protein tag; and ii) an antibody specific for a D2 dopamine receptor.

### Self-Labelling Polypeptides

Suitable self-labelling polypeptides (also referred to herein as “self-labelling protein tags”) include a SNAP polypeptide, a CLIP polypeptide, or a HALO polypeptide. Also suitable for use is a halo-based oligonucleotide binder (HOB) polypeptide. See, e.g., Kossman et al. (2016) Chembiochem. 17:1102. A HOB polypeptide binds chlorohexyl moieties. Also suitable for use is a trimethoprim (TMP) tag, an engineered form of E. coli dihydrofolate reductase (DHFR) that forms a non-covalent high-affinity complex with trimethoprim derivatives. See, e.g., Gallagher et al. (2009) ACS Chem. Biol. 4:547; and Jing and Cornish (2013) ACS Chem. Biol. 8:1704.

A SNAP polypeptide can comprise an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the following amino acid sequence:

(SEQ ID NO: 1)
MDKDCEMKRTTLDSPLGKLELSGCEQGLHRIIFLGKGTSAADAVEVPAP
AAVLGGPEPLMQATAWLNAYFHQPEAIEEFPVPALHHPVFQQESFTRQV
LWKLLKVVKFGEVISYSHLAALAGNPAATAAVKTALSGNPVPILIPCHR
VVQGDLDVGGYEGGLAVKEWLLAHEGHRLGKPGLG. A SNAP
polypeptide binds O⁶-benzylguanine (BG).

A CLIP polypeptide can comprise an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the following amino acid sequence:

(SEQ ID NO: 2)
MDKDCEMKRTTLDSPLGKLELSGCEQGLHRIIFLGKGTSAADAVEVPAP
AAVLGGPEPLIQATAWLNAYFHQPEAIEEFPVPALHHPVFQQESFTRQV
LWKLLKVVKFGEVISESHLAALVGNPAATAAVNTALDGNPVPILIPCHR
VVQGDSDVGPYLGGLAVKEWLLAHEGHRLGKPGLG. A CLIP
polypeptide can bind O²-benzylcytosine (BC).

A HALO polypeptide can comprise an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the following amino acid sequence:

(SEQ ID NO: 3)
MAEIGTGFPFDPHYVEVLGERMHYVDVGPRDGTPVLFLHGNPTSSYVWR
NIIPHVAPTHRCIAPDLIGMGKSDKPDLGYFFDDHVRFMDAFIEALGLE
EVVLVIHDWGSALGFHWAKRNPERVKGIAFMEFIRPIPTWDEWPEFARE
TFQAFRTTDVGRKLIIDQNVFIEGTLPMGVVRPLTEVEMDHYREPFLNP
VDREPLWRFPNELPIAGEPANIVALVEEYMDWLHQSPVPKLLFWGTPGV
LIPPAEAARLAKSLPNCKAVDIGPGLNLLQEDNPDLIGSEIARWLSTLE
ISG. A HALO polypeptide binds chloroalkane.

### Membrane Anchors

Suitable membrane anchors include polypeptides that insert into a eukaryotic cell plasma membrane (e.g., a mammalian cell plasma membrane). Suitable membrane anchors include single-pass transmembrane polypeptides. The membrane anchor may span the entire plasma membrane, but need not do so. The membrane anchor can be any natural or artificial transmembrane domain may comprise a hydrophobic a-helix of about 20 amino acids. Prediction of transmembrane domains/segments may be made using publicly available prediction tools (e.g. TMHMM, Krogh et al. (2001) J. Molec. Biol. 305(3):567-580; and TMpred, Hofmann and Stoffel (1993) Biol. Chem. Hoppe-Seyler 347:166).

The transmembrane domain of any polypeptide can be used as the membrane anchor. Non-limiting examples include, e.g., the membrane anchor of glycophorin A (GPA), the membrane anchor of small integral membrane protein 1 (SMIM1), a platelet derived growth factor receptor (PDGFR) transmembrane domain, and the like.

### Endoplasmic Reticulum Export Signals

In some cases, a fusion polypeptide includes: i) a self-labelling polypeptide; ii) a peptide linker; iii) a membrane anchoring polypeptide; and iv) an endoplasmic reticulum (ER) export signal peptide.

Suitable ER export signal peptides include, e.g., VXXSL (SEQ ID NO:4) (where X is any amino acid) (e.g., VKESL (SEQ ID NO:5); VLGSL (SEQ ID NO:6); etc.); NANSFCYENEVALTSK (SEQ ID NO:7); FXYENE (SEQ ID NO:8) (where X is any amino acid), e.g., FCYENEV (SEQ ID NO:9); and the like. An ER export signal peptide can have a length of from about 5 amino acids to about 25 amino acids, e.g., from about 5 amino acids to about 10 amino acids, from about 10 amino acids to about 15 amino acids, from about 15 amino acids to about 20 amino acids, or from about 20 amino acids to about 25 amino acids.

### Peptide Linkers

As noted above, in some cases, a fusion polypeptide includes: i) a self-labelling polypeptide; ii) a peptide linker; iii) a membrane anchoring polypeptide. In some cases, the peptide linker is a rigid linker, such as a peptide of the formula: [A(EAAAK)nA]x (SEQ ID NO:10) (where n=2-4 and x=1 or 2). Another example of a suitable rigid linker is a peptide of the formula: (XP)n (SEQ ID NO:11), where X is any amino acid (e.g., where X is Ala, Lys, or Glu), and where n is 1, 2, 3, 4, 5, 6, 7, 8, 9, or 10. In some cases, the peptide linker comprises the amino acid sequence (EAAAK)n (SEQ ID NO:12), where n is an integer from 1 to 6.

### Antibody to D2 Dopamine Receptor

As noted above, in some cases, a fusion polypeptide includes: i) a self-labelling polypeptide; and ii) an antibody specific for a D2 dopamine receptor. In some cases, the anti-D2 dopamine receptor antibody is a nanobody. In some cases, the anti-D2 dopamine receptor antibody is a scFv.

### Nucleic Acids

As noted above, a system of the present disclosure can include a fusion polypeptide per se, as described above, or can include a nucleic acid (e.g., an expression vector) comprising a nucleotide sequence encoding the fusion polypeptide.

The nucleic acid can be an expression vector. The nucleotide sequence can be operably linked to a promoter. The expression vector can be, e.g., a recombinant viral expression vector (e.g., a recombinant adenovirus-associated virus vector; a recombinant retroviral vector; and the like). The nucleotide sequence encoding the fusion polypeptide can be operably linked to one or more transcriptional control elements (e.g., promoters; enhancers). For example, the nucleotide sequence encoding the fusion polypeptide can be operably linked to one or more transcriptional control elements that provide for preferential expression in a target cell type. As an example, the nucleotide sequence encoding the fusion polypeptide can be operably linked to one or more transcriptional control elements that provide for preferential expression in a D2 dopamine receptor. For example, the nucleotide sequence encoding the fusion polypeptide can be operably linked to D2 dopamine receptor promoter and enhancer elements.

### Conjugates

The present disclosure provides a conjugate, wherein the conjugate is a compound of Formula I:

DRL-HP-L-HT (I)

wherein:
DRL is a dopamine receptor ligand;
HP is a hydrophobic moiety;
L is a linker; and
H is a halogen-containing substrate.

In some embodiments, the dopamine receptor ligand (DRL) comprises an aminoindenyl moiety. In some instances, the dopamine receptor ligand comprises a N-propyl 2-aminoindenyl moiety. In some embodiments, the dopamine receptor ligand comprises a phenylpiperazine moiety. In some instances, the dopamine receptor ligand comprises a halo-substituted phenylpiperazine moiety. In some instances, the dopamine receptor ligand comprises an alkoxy substituted phenylpiperazine moiety, such as a methoxy substituted phenylpiperazine moiety. In some embodiments, the hydrophobic moiety (HP) comprises an azobenzene moiety. In some embodiments, the linker comprises a polyalkylene glycol, such as a polyethylene glycol where the polyalkylene glycol has n polymer units of 8 or more, such as 9, 10, 11, 12, 13, 14, 15, 16, 17, 18, 19, 20, 21, 22, 23 and including where the polyalkylene glycol has n polymer units of 24 or more. In certain instances, the linker comprises a polyethylene glycol having 24 polymer units. In some embodiments, the linker comprises a heterobifunctional component, such as a bis-amide component. In certain embodiments, the linker comprises a heterobifunctional terminated polyalkylene glycol. In some instances, the linker comprises a heterobifunctional, carboxylic acid-terminated polyethylene glycol, such as a heterobifunctional, carboxylic acid-terminated PEG[24]. In some embodiments, the halogen-containing substrate is a haloalkane substrate, such as a chloroalkane substrate.

In some embodiments, a conjugate of the present disclosure is a compound of Formula IA:

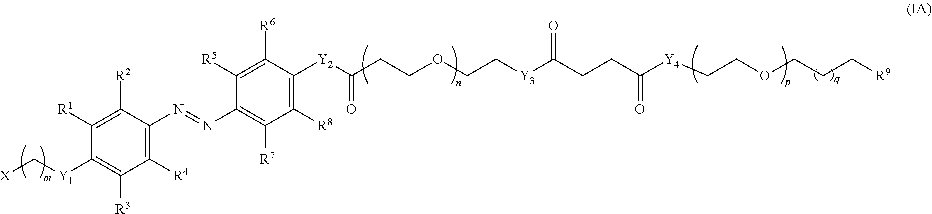

wherein X is:

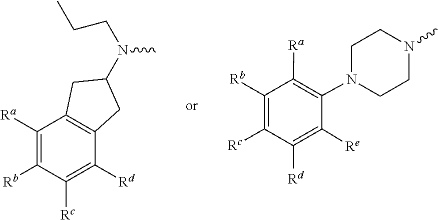

wherein [symbol] represents the X—(C)ₘ— bond;
m is an integer from 1-10;
n is an integer from 1-50;
p is an integer from 1-10;
q is an integer from 1-25;
Y₁, Y₂, Y₃ and Y₄ are independently selected from C, N, O or S;
R^{a}, R^{b}, R^{c} and R^{d} are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
R¹, R², R³, R⁴, R⁵, R⁶, R⁷ and R⁸ are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
R⁹ is selected from hydrogen, halogen, alkoxy, nitro, amino, hydroxy, cyano, thiol.

In some embodiments, one or more of R^{a}, R^{b}, R^{c} and R^{d} is independently selected from F, Cl, Br and I. In certain instances, one or more of R^{a}, R^{b}, R^{c} and R^{d} is Cl. In certain embodiments, R^{a} and R^{b} are Cl. In some embodiments, one or more of R^{a}, R^{b}, R^{c} and R^{d} is a C(1-6) alkoxy, such as methoxy or ethoxy. In certain instances, R^{a} is methoxy.

In some embodiments, R¹, R², R³, R⁴, R⁵, R⁶, R⁷ and R⁸ are hydrogen. In some embodiments, one or more of R¹, R², R³, R⁴, R⁵, R⁶, R⁷ and R⁸ is a C(1-8) alkyl, such as a C(1-8) linear alkyl or C(1-8) branched alkyl. For example, one or more of R¹, R², R³, R⁴, R⁵, R⁶, R⁷ and R⁸ may be methyl, ethyl, n-propyl, isopropyl, n-butyl, isobutyl, pentyl, isopentyl, hexyl, heptyl, octyl.

In certain embodiments, m is an integer from 2-8, such as from 2-6. In certain instances, m is 4. In certain instances, m is 3.

In certain embodiments, n is an integer from 12-30, such as from 16-28. In certain instances, n is 24.

In certain embodiments, p is an integer from 2-8, such as from 2-6. In certain instances, p is 2.

In certain embodiments, q is an integer from 2-12, such as from 2-8. In certain instances, q is 4.

In certain embodiments, R⁹ is selected from F, Cl, Br and I. In certain instances, R⁹ is Cl.

In certain embodiments, the dopamine receptor ligand is a compound of Formula 1A:

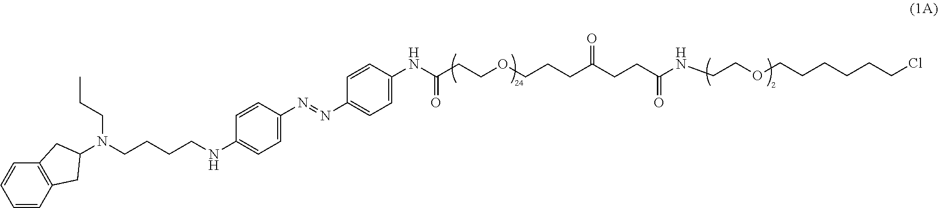

In certain embodiments, the dopamine receptor ligand is a compound of Formula 1B:

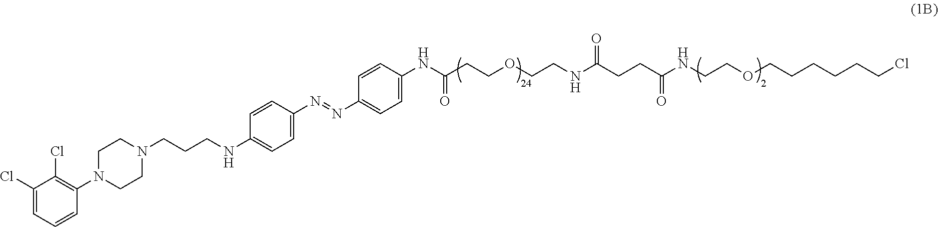

In certain embodiments, the conjugate is a compound of Formula 1C:

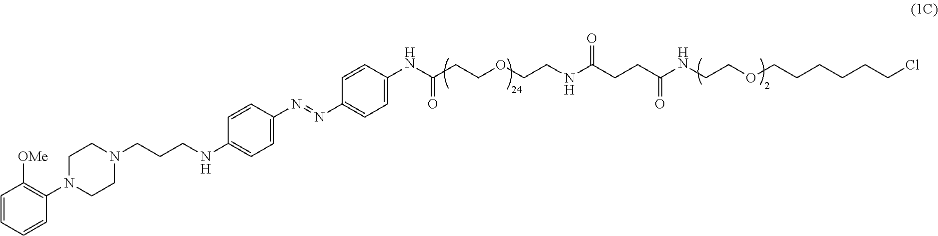

In some embodiments, a conjugate of the present disclosure is a compound of Formula IIA:

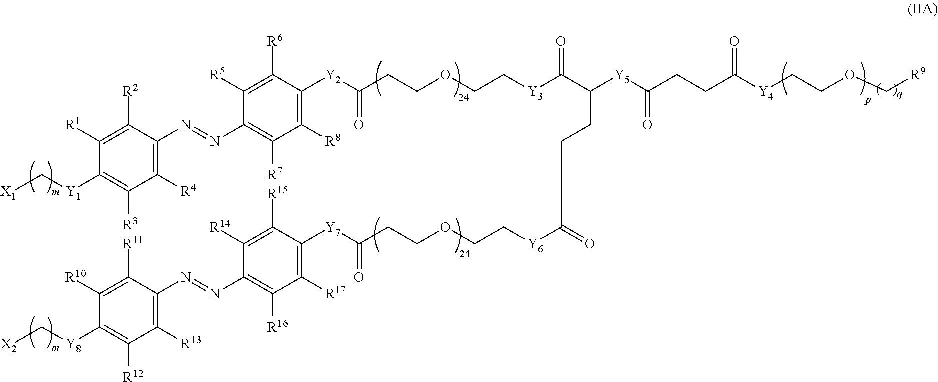

wherein X₁ and X₂ are independently selected from:

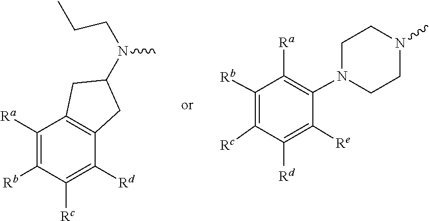

wherein [symbol] represents the X—(C)ₘ— bond;
m is an integer from 1-10;
n is an integer from 1-50;
p is an integer from 1-10;
q is an integer from 1-25;
Y₁, Y₂, Y₃, Y₄, Y₅, Y₆, Y₇ and Y₈ are independently selected from C, N, O or S;
R^{a}, R^{b}, R^{c} and R^{d} are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
R¹, R², R³, R⁴, R⁵, R⁶, R⁷, R⁸, R¹⁰, R¹¹, R¹², R¹³, R¹⁴, R¹⁵, R¹⁶ and R¹⁷ are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
R⁹ is selected from hydrogen, halogen, alkoxy, nitro, amino, hydroxy, cyano, thiol.

In some embodiments, one or more of R^{a}, R^{b}, R^{c} and R^{d} is independently selected from F, Cl, Br and I. In certain instances, one or more of R^{a}, R^{b}, R^{c} and R^{d} is Cl. In certain embodiments, R^{a} and R^{b} are Cl. In some embodiments, one or more of R^{a}, R^{b}, R^{c} and R^{d} is a C(1-6) alkoxy, such as methoxy or ethoxy. In certain instances, R^{a} is methoxy.

In some embodiments, R¹, R², R³, R⁴, R⁵, R⁶, R⁷, R⁸, R¹⁰, R¹¹, R¹², R¹³, R¹⁴, R¹⁵, R¹⁶ and R¹⁷ are hydrogen. In some embodiments, one or more of R¹, R², R³, R⁴, R⁵, R⁶, R⁷, R⁸, R¹⁰, R¹¹, R¹², R¹³, R¹⁴, R¹⁵, R¹⁶ and R¹⁷ are is a C(1-8) alkyl, such as a C(1-8) linear alkyl or C(1-8) branched alkyl. For example, one or more of R¹, R², R³, R⁴, R⁵, R⁶, R⁷, and R⁸ may be methyl, ethyl, n-propyl, isopropyl, n-butyl, isobutyl, pentyl, isopentyl, hexyl, heptyl, octyl.

In certain embodiments, m is an integer from 2-8, such as from 2-6. In certain instances, m is 4. In certain instances, m is 3.

In certain embodiments, n is an integer from 12-30, such as from 16-28. In certain instances, n is 24.

In certain embodiments, p is an integer from 2-8, such as from 2-6. In certain instances, p is 2.

In certain embodiments, q is an integer from 2-12, such as from 2-8. In certain instances, q is 4.

In certain embodiments, R⁹ is selected from F, Cl, Br and I. In certain instances, R⁹ is Cl.

In some embodiments, a conjugate of the present disclosure is a compound of Formula 2A:

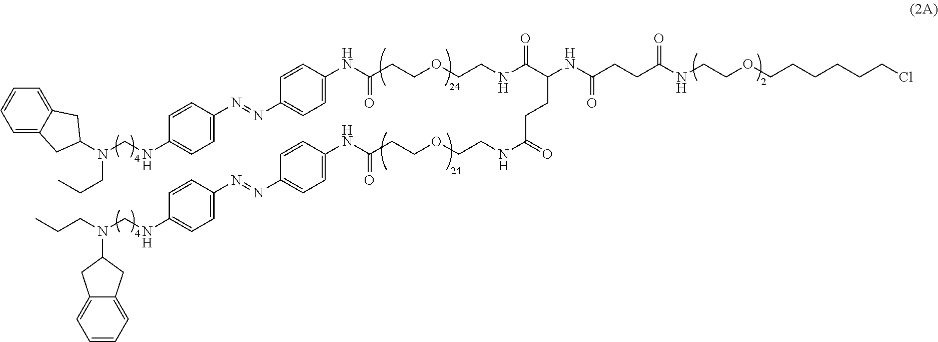

In some cases, a conjugate of the present disclosure has the following structure:

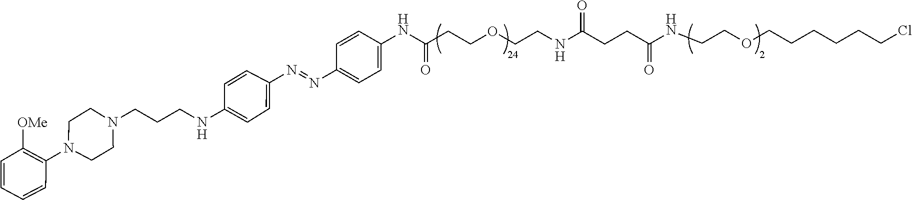

As another example, in some cases, a conjugate of the present disclosure has the following structure:

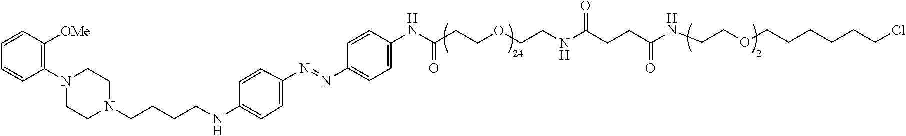

As another example, in some cases, a conjugate of the present disclosure has the following structure:

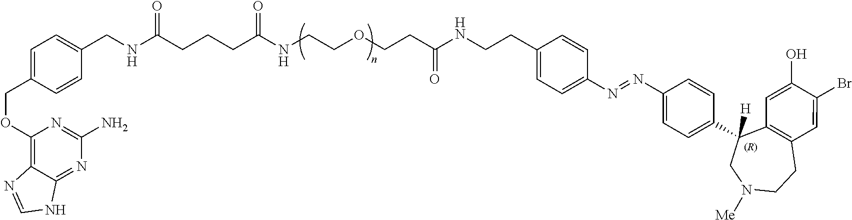

### Compositions

The present disclosure provides compositions, including pharmaceutical compositions, comprising a system or a conjugate of the present disclosure.

Compositions comprising a system of the present disclosure or a conjugate of the present disclosure can include one or more of: a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-morpholino)ethanesulfonic acid (MES), 2-(N-morpholino)ethanesulfonic acid sodium salt (MES), 3-(N-Morpholino)propanesulfonic acid (MOPS), N-tris[hydroxymethyl]methyl-3-aminopropanesulfonic acid (TAPS), etc.; a solubilizing agent; a detergent, e.g., a non-ionic detergent such as Tween-20, Nonidet-P40, etc.; a protease inhibitor; and the like.

The present disclosure provides pharmaceutical compositions comprising a conjugate of the present disclosure. In some cases, the pharmaceutical composition is suitable for administering to an individual in need thereof. In some cases, the pharmaceutical composition is suitable for administering to an individual in need thereof, where the individual is a human.

A pharmaceutical composition comprising a system or a conjugate of the present disclosure may be administered to a patient alone, or in combination with other supplementary active agents. The pharmaceutical compositions may be manufactured using any of a variety of processes, including, without limitation, conventional mixing, dissolving, granulating, levigating, emulsifying, encapsulating, entrapping, and lyophilizing. The pharmaceutical composition can take any of a variety of forms including, without limitation, a sterile solution, suspension, emulsion, lyophilizate, tablet, pill, pellet, capsule, powder, syrup, elixir or any other dosage form suitable for administration.

A pharmaceutical composition comprising a system or a conjugate of the present disclosure can optionally include a pharmaceutically acceptable carrier(s) that facilitate processing of an active ingredient into pharmaceutically acceptable compositions. As used herein, the term “pharmacologically acceptable carrier” refers to any carrier that has substantially no long-term or permanent detrimental effect when administered and encompasses terms such as “pharmacologically acceptable vehicle, stabilizer, diluent, auxiliary or excipient.” Such a carrier generally is mixed with an active compound, or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., distilled, deionized water, saline; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in “Pharmaceutical Dosage Forms and Drug Delivery Systems” (Howard C. Ansel et al., eds., Lippincott Williams & Wilkins Publishers, 7^{th} ed. 1999); “Remington: The Science and Practice of Pharmacy” (Alfonso R. Gennaro ed., Lippincott, Williams & Wilkins, 20^{th} 2000); “Goodman & Gilman's The Pharmacological Basis of Therapeutics” Joel G. Hardman et al., eds., McGraw-Hill Professional, 10.sup.th ed. 2001); and “Handbook of Pharmaceutical Excipients” (Raymond C. Rowe et al., APhA Publications, 4^{th} edition 2003).

A subject pharmaceutical composition can optionally include, without limitation, other pharmaceutically acceptable components, including, without limitation, buffers, preservatives, tonicity adjusters, salts, antioxidants, physiological substances, pharmacological substances, bulking agents, emulsifying agents, wetting agents, sweetening or flavoring agents, and the like. Various buffers and means for adjusting pH can be used to prepare a pharmaceutical composition disclosed in the present specification, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate and a stabilized oxy chloro composition, for example, PURITE™. Tonicity adjustors suitable for inclusion in a subject pharmaceutical composition include, without limitation, salts such as, e.g., sodium chloride, potassium chloride, mannitol or glycerin and other pharmaceutically acceptable tonicity adjustor. It is understood that these and other substances known in the art of pharmacology can be included in a subject pharmaceutical composition.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

A conjugate of the present disclosure can be formulated with one or more pharmaceutically acceptable excipients. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In a method of the present disclosure (described below), a conjugate of the present disclosure may be administered to the host using any convenient means capable of resulting in the desired reduction in disease condition or symptom. Thus, a conjugate of the present disclosure can be incorporated into a variety of formulations for therapeutic administration. More particularly, a conjugate of the present disclosure can be formulated into pharmaceutical compositions by combination with appropriate pharmaceutically acceptable carriers or diluents, and may be formulated into preparations in solid, semisolid, liquid or gaseous forms, such as tablets, capsules, powders, granules, ointments, solutions, suppositories, injections, inhalants and aerosols.

A conjugate of the present disclosure can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Formulations suitable for injection can be administered by an intravitreal, intraocular, intramuscular, subcutaneous, sublingual, or other route of administration, e.g., injection into the gum tissue or other oral tissue. Such formulations are also suitable for topical administration.

A system or a conjugate of the present disclosure can be administered as injectables. Injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

A system or a conjugate of the present disclosure can be formulated in a pharmaceutical composition together with a pharmaceutically acceptable excipient. In some cases, a subject pharmaceutical composition will be suitable for administration to a subject, e.g., will be sterile. For example, in some cases, a subject pharmaceutical composition will be suitable for administration to a human subject, e.g., where the composition is sterile and is free of detectable pyrogens and/or other toxins.

In some cases, a system or a conjugate of the present disclosure is delivered by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

### Methods

The present disclosure provides a method of modulating the activity of a target ligand-binding polypeptide (e.g., a D2 dopamine receptor), the method comprising contacting a cell comprising the target ligand-binding polypeptide (e.g., the D2 dopamine receptor) with a system of the present disclosure or a conjugate of the present disclosure. The present disclosure provides a method of modulating the activity of a target ligand-binding polypeptide (e.g., a D2 dopamine receptor), the method comprising: a) contacting a cell comprising the target ligand-binding polypeptide (e.g., the D2 dopamine receptor) with a system of the present disclosure or a conjugate of the present disclosure; and b) exposing the cell to light of a wavelength that isomerizes the photoisomerizable agent present in the conjugate. In some cases, the cell is present in an individual. For example, in some cases, the cell comprising the D2 dopamine receptor is in the dorsal striatum of an individual. In some cases, the cell is a direct pathway medium spiny neuron, and the ligand present in the conjugate is dopamine or a dopamine derivative or analog, where the ligand functions as a D2 dopamine receptor agonist.

The present disclosure provides a method of modulating the activity of a D2 dopamine receptor, including the G_{i/o/z}-coupled D2-like receptors D2R and D3R. See, e.g., Dal Toso et al. (1989) EMBO J. 8:4025. The present disclosure provides a method of modulating the activity of a D1 dopamine receptor, including the D1-like receptors D1R and D5R. In some cases, the D2 dopamine receptor and/or the D1 dopamine receptor is in a neuron, e.g., a direct pathway medium spiny neuron. In some cases, a method of the present disclosure comprises contacting a D2 dopamine receptor with a conjugate of the present disclosure, or a system of the present disclosure, where the DRL is a D2 dopamine receptor agonist. In some cases, a method of the present disclosure comprises contacting a D2 dopamine receptor with a conjugate of the present disclosure, or a system of the present disclosure, where the DRL is a D2 dopamine receptor antagonist. In some cases, a method of the present disclosure comprises contacting a D1 dopamine receptor with a conjugate of the present disclosure, or a system of the present disclosure, where the DRL is a D1 dopamine receptor antagonist. In some cases, a method of the present disclosure comprises: a) contacting a D2 dopamine receptor with a conjugate of the present disclosure, or a system of the present disclosure, where the DRL is a D2 dopamine receptor agonist; and b) contacting a D1 dopamine receptor with a conjugate of the present disclosure, or a system of the present disclosure, where the DRL is a D1 dopamine receptor antagonist.

The present disclosure provides a method of treating Parkinson's disease in an individual, the method comprising administering a system of the present disclosure, or a conjugate of the present disclosure, into the dorsal striatum of the individual. The present disclosure provides a method of treating Parkinson's disease in an individual, the method comprising: a) administering a system of the present disclosure, or a conjugate of the present disclosure, into the dorsal striatum of the individual; and b) exposing the dorsal striatum to light of a wavelength that isomerizes the photoisomerizable agent present in the conjugate, such that the ligand (dopamine or a dopamine derivative or analog, where the ligand functions as a D2 dopamine receptor agonist) binds to the D2 dopamine receptor. In some cases, the light is provided by an implantable light source. Administration of a system or a conjugate of the present disclosure can provide for an increase in motor function in an individual in need thereof.

Implantable light sources for use in delivering light to the brain of an individual are known in the art. See, e.g., Rossi et al. (2015) Front. Integr. Neurosci. 9:8; and McAlinden et al. (2019) Neutophotonics 6:035010. A suitable light source can include an implantable light-emitting diode (see Rossi et al. (2015) Front. Integr. Neurosci. 9:8). A suitable light source can include an implantable LED; and a control device for controlling light output by the LED.

The present disclosure provides a method of treating schizophrenia in an individual, the method comprising administering a system of the present disclosure, or a conjugate of the present disclosure.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A system comprising:
a1) a conjugate of Formula I:

DRL-HP-L-HT (I)
wherein:
   DRL is a dopamine receptor ligand;
   HP is a hydrophobic moiety;
   L is a linker; and
H is a halogen-containing substrate; and
b1) a fusion polypeptide, or a recombinant expression vector comprising a nucleotide sequence encoding the fusion polypeptide, wherein the fusion polypeptide comprises:
i) a self-labeling protein tag;
ii) a peptide linker; and
iii) a membrane-anchoring polypeptide; or
a2) a conjugate of Formula I:

DRL-HP-L-HT (I)
wherein:
   DRL is a dopamine receptor ligand;
   HP is a hydrophobic moiety;
   L is a linker; and
H is a halogen-containing substrate; and
b2) a fusion polypeptide, or a recombinant expression vector comprising a nucleotide sequence encoding the fusion polypeptide, wherein the fusion polypeptide comprises:
i) a self-labeling protein tag; and
ii) an antibody specific for a D2 dopamine receptor.

Aspect 2. The system of aspect 1, wherein H comprises benzylguanine, chloroalkane, or benzylcytosine.

Aspect 3. The system of aspect 1 or aspect 2, wherein DRL comprises an aminoindenyl moiety, a N-propyl 2-aminoindenyl moiety, phenylpiperazine moiety, a halo-substituted phenylpiperazine moiety, or a methoxy substituted phenylpiperazine moiety.

Aspect 4. The system of any one of aspects 1-3, wherein HP comprises a moiety selected from an azobenzene, a cyclic azobenzene, an azoheteroarene, a fulgide, a spiropyran, a triphenyl methane, a thioindigo, a diarylethene, and an overcrowded alkene.

Aspect 5. The system of any one of aspects 1-3, wherein HP comprises an azobenzene moiety.

Aspect 6. The system of any one of aspects 1-5, wherein DRL is a D2 dopamine receptor ligand.

Aspect 7. The system of any one of aspects 1-6, wherein the linker comprises a polyalkylene glycol.

Aspect 8. The system of aspect 7, wherein the polyalkylene glycol is poly(ethylene glycol) (PEG).

Aspect 9. The system of aspect 8, wherein the linker comprises (PEG)n, where n is an integer from 8 to 24.

Aspect 10. The system of any one of aspects 1-8, wherein HP comprises an azobenzene moiety that isomerizes in response to visible light.

Aspect 11. The system of aspect 6, wherein the DRL functions as a D2 dopamine receptor agonist.

Aspect 12. The system of aspect 6, wherein the DRL functions as a positive allosteric modulator of the D2 dopamine receptor.

Aspect 13. The system of any one of aspects 1-12, wherein the conjugate is a compound of Formula IA:

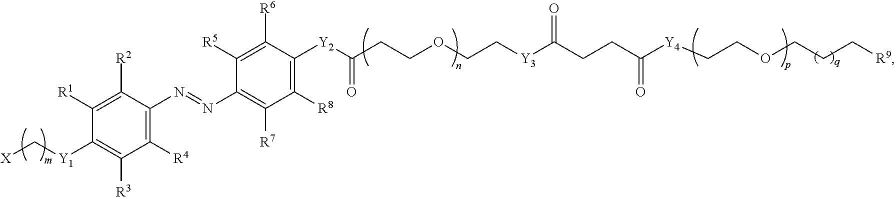

wherein X is:

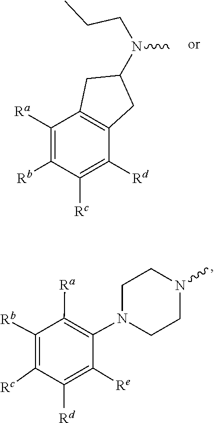

wherein [symbol] represents the X—(C)ₘ— bond;
m is an integer from 1-10;
n is an integer from 1-50;
p is an integer from 1-10;
q is an integer from 1-25;
Y₁, Y₂, Y₃ and Y₄ are independently selected from C, N, O or S;
R^{a}, R^{b}, R^{c} and R^{d} are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
R¹, R², R³, R⁴, R⁵, R⁶, R⁷ and R⁸ are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
R⁹ is selected from hydrogen, halogen, alkoxy, nitro, amino, hydroxy, cyano, thiol.

Aspect 14. The system of aspect 13, wherein:
a) one or more of R^{a}, R^{b}, R^{c} and R^{d} is independently selected from F, Cl, Br and I; or
b) one or more of R^{a}, R^{b}, R^{c} and R^{d} is a C(1-6) alkoxy, such as methoxy or ethoxy.

Aspect 15. The system of any one of aspects 1-14, wherein:
a) the conjugate is a compound of Formula 1A:

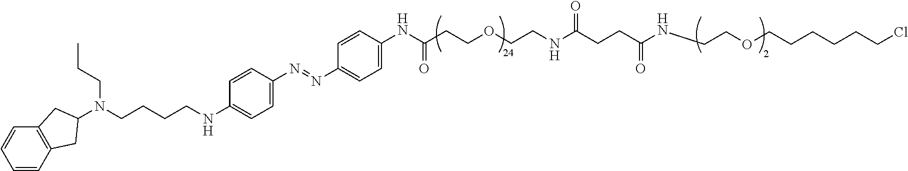

b) the conjugate is a compound of Formula 1B:

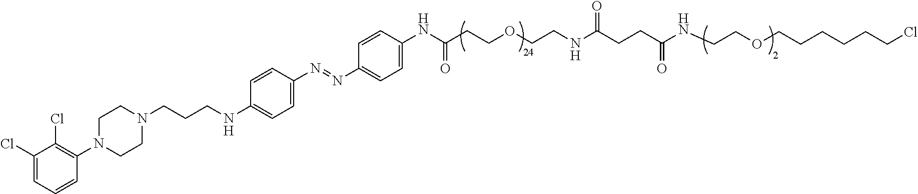

or
c) the conjugate is a compound of Formula 1C:

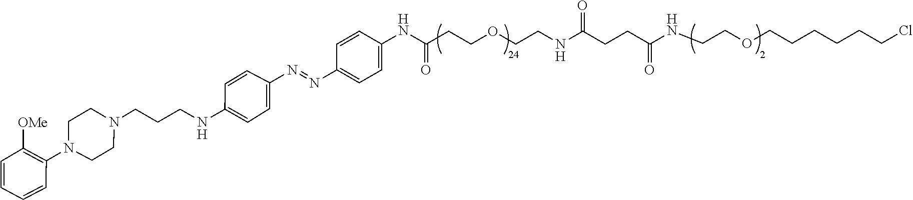

Aspect 16. The system of any one of aspects 1-14, wherein the conjugate is a compound of Formula IIA:

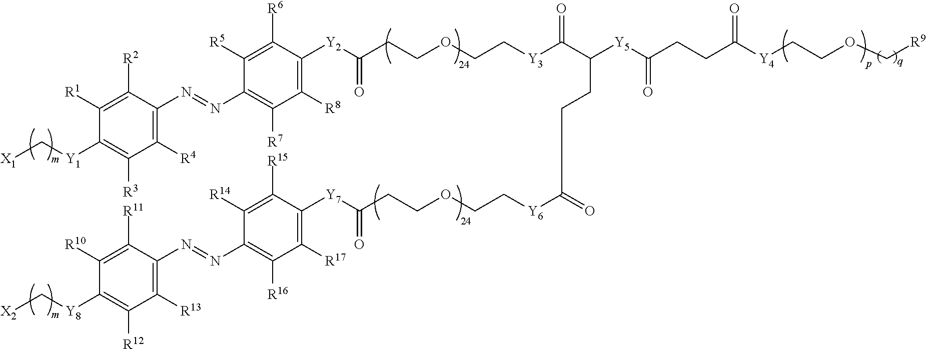

wherein X₁ and X₂ are independently selected from:

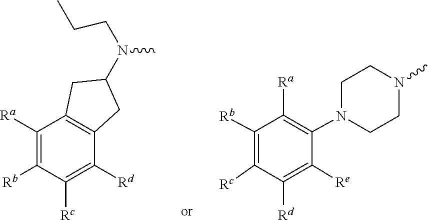

wherein [symbol] represents the X—(C)ₘ— bond;
   m is an integer from 1-10;
n is an integer from 1-50;
p is an integer from 1-10;
q is an integer from 1-25;
Y₁, Y₂, Y₃, Y₄, Y₅, Y₆, Y₇ and Y₈ are independently selected from C, N, O or S;
R^{a}, R^{b}, R^{c} and R^{d} are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
R¹, R², R³, R⁴, R⁵, R⁶, R⁷, R⁸, R¹⁰, R¹¹, R¹², R¹³, R¹⁴, R¹⁵, R¹⁶ and R¹⁷ are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
R⁹ is selected from hydrogen, halogen, alkoxy, nitro, amino, hydroxy, cyano, thiol.

Aspect 17. The system of aspect 16, wherein:
a) one or more of R^{a}, R^{b}, R^{c} and R^{d} is independently selected from F, Cl, Br and I; or
b) one or more of R^{a}, R^{b}, R^{c} and R^{d} is a C(1-6) alkoxy, such as methoxy or ethoxy.

Aspect 18. The system of aspect 17, wherein the conjugate is a compound of Formula 2A:

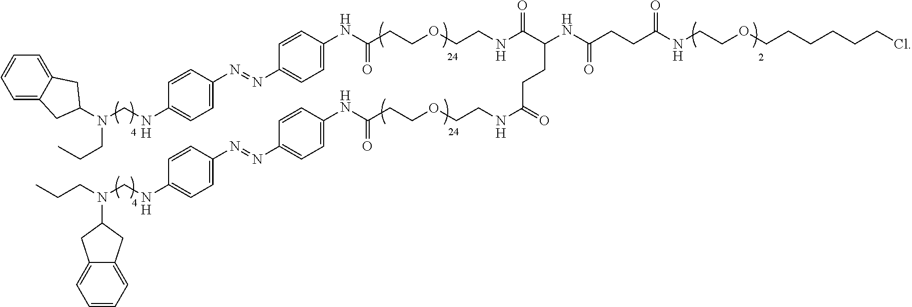

Aspect 19. The system of aspect 1, wherein the DRL is a D2 dopamine receptor antagonist.

Aspect 20. The system of aspect 19, wherein the conjugate has the following structure:

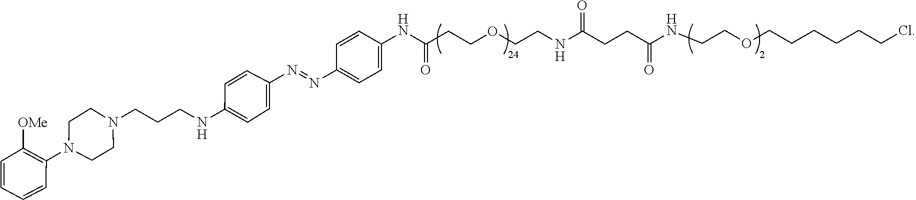

Aspect 21. The system of aspect 19, wherein the conjugate has the following structure:

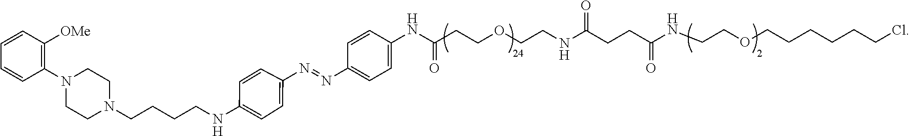

Aspect 22. The system of aspect 1, wherein the DRL is a D1 dopamine receptor ligand.

Aspect 23. The system of aspect 1, wherein the DRL is a D1 dopamine receptor antagonist.

Aspect 24. The system of aspect 23, wherein the conjugate has the following structure:

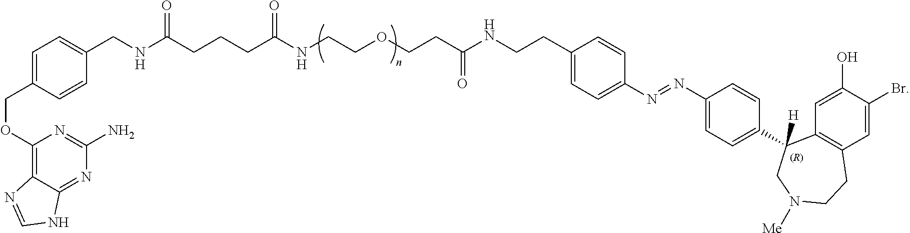

Aspect 25. The system of any one of aspects 1-24, wherein the self-labeling protein tag comprises:
a) an amino acid sequence having at least 80% amino acid sequence identity to the SNAP polypeptide amino acid sequence set forth in SEQ ID NO:1;
b) an amino acid sequence having at least 80% amino acid sequence identity to the CLIP polypeptide amino acid sequence set forth in SEQ ID NO:2; or
c) an amino acid sequence having at least 80% amino acid sequence identity to the HALO polypeptide amino acid sequence set forth in SEQ ID NO:3.

Aspect 26. The system of any one of aspects 1-25, wherein the fusion polypeptide comprises an endoplasmic reticulum (ER) export signal peptide.

Aspect 27. The system of any one of aspects 1-26, wherein the peptide linker comprises the amino acid sequence EAAAK (SEQ ID NO:13).

Aspect 28. The system of any one of aspects 1-27, wherein the nucleotide sequence encoding the fusion polypeptide is operably linked to a cell type-specific promoter.

Aspect 29. The system of aspect 28, wherein the promoter is a dopamine-1 receptor promoter.

Aspect 30. The system of any one of aspects 1-29, where the antibody is a nanobody or a single-chain Fv.

Aspect 31. A method of modulating the activity of a D2 dopamine receptor, the method comprising:
a) contacting a cell comprising the D2 dopamine receptor with a system of any one of aspects 1-30; and
b) exposing the cell to light of a wavelength that isomerizes the photoisomerizable agent present in the conjugate.

Aspect 32. The method of aspect 31, wherein the cell is in an individual.

Aspect 33. The method of aspect 31 or aspect 32, wherein the light is provided by an implantable light source.

Aspect 34. The method of any one of aspects 31-33, wherein the cell is a direct pathway medium spiny neuron.

Aspect 35. A method of treating Parkinson's disease in an individual, the method comprising administering the system of any one of aspects 1-30 into the dorsal striatum of the individual.

Aspect 36. The method of aspect 35, comprising exposing the dorsal striatum to light of a wavelength that isomerizes the photoisomerizable agent present in the conjugate.

Aspect 37. The method of aspect 35 or 36, wherein the light is provided by an implantable light source.

Aspect 38. A compound of Formula I:

DRL-HP-L-HT (I)
wherein:
   DRL is a dopamine receptor ligand;
   HP is a hydrophobic moiety;
   L is a linker; and
H is a halogen-containing substrate.

Aspect 39. The compound of aspect 38, wherein H comprises benzylguanine, chloroalkane, or benzylcytosine.

Aspect 40. The compound of aspect 38 or aspect 39, wherein DRL comprises an aminoindenyl moiety, a N-propyl 2-aminoindenyl moiety, phenylpiperazine moiety, a halo-substituted phenylpiperazine moiety, or a methoxy substituted phenylpiperazine moiety.

Aspect 41. The compound of any one of aspects 38-40, wherein HP comprises a moiety selected from an azobenzene, a cyclic azobenzene, an azoheteroarene, a fulgide, a spiropyran, a triphenyl methane, a thioindigo, a diarylethene, and an overcrowded alkene.

Aspect 42. The compound of any one of aspects 38-41, wherein HP comprises an azobenzene moiety.

Aspect 43. The compound of any one of aspects 38-42, wherein DRL is a D2 dopamine receptor ligand.

Aspect 44. The compound of any one of aspects 38-43, wherein the linker comprises a polyalkylene glycol.

Aspect 45. The compound of aspect 44, wherein the polyalkylene glycol is poly(ethylene glycol) (PEG).

Aspect 46. The compound of aspect 45, wherein the linker comprises (PEG)n, where n is an integer from 8 to 24.

Aspect 47. The compound of any one of aspects 38-46, wherein HP comprises an azobenzene moiety that isomerizes in response to visible light.

Aspect 48. The compound of aspect 43, wherein the DRL functions as a D2 dopamine receptor agonist.

Aspect 49. The compound of aspect 43, wherein the DRL functions as a positive allosteric modulator of the D2 dopamine receptor.

Aspect 50. The compound of any one of aspects 38-49, wherein the compound is of Formula IA:

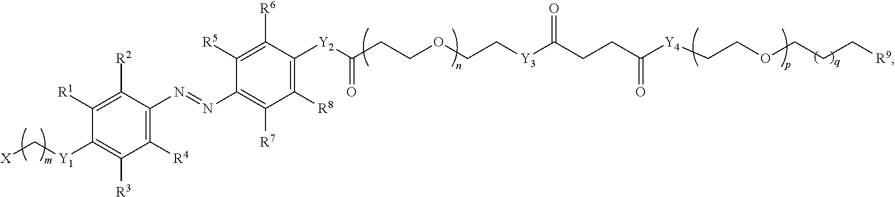

wherein X is:

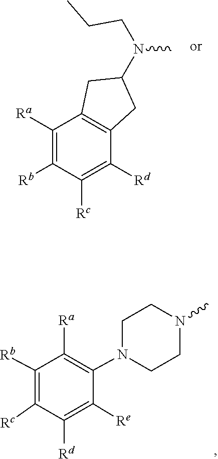

wherein [symbol] represents the X—(C)ₘ— bond;
m is an integer from 1-10;
n is an integer from 1-50;
p is an integer from 1-10;
q is an integer from 1-25;
Y₁, Y₂, Y₃ and Y₄ are independently selected from C, N, O or S;
R^{a}, R^{b}, R^{c} and R^{d} are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
R¹, R², R³, R⁴, R⁵, R⁶, R⁷ and R⁸ are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
R⁹ is selected from hydrogen, halogen, alkoxy, nitro, amino, hydroxy, cyano, thiol.

Aspect 51. The compound of aspect 50, wherein:
a) one or more of R^{a}, R^{b}, R^{c} and R^{d} is independently selected from F, Cl, Br and I; or
b) one or more of R^{a}, R^{b}, R^{c} and R^{d} is a C(1-6) alkoxy, such as methoxy or ethoxy.

Aspect 52. The compound of any one of aspects 38-51, wherein:
a) the conjugate is a compound of Formula 1A:

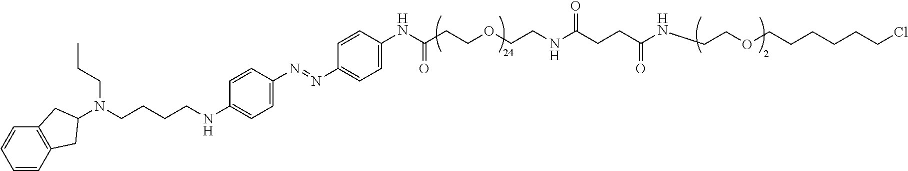

b) the conjugate is a compound of Formula 1B:

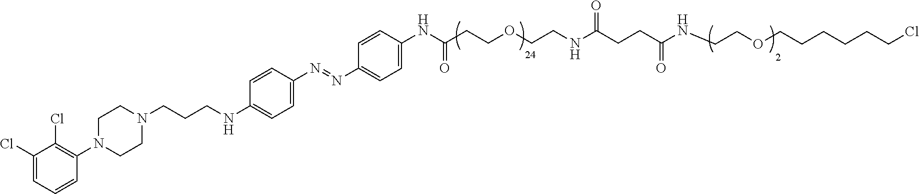

or
c) the conjugate is a compound of Formula 1C:

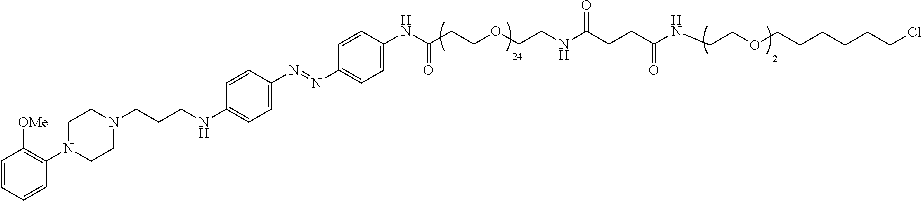

Aspect 53. The compound of any one of aspects 38-49, wherein the conjugate is a compound of Formula IIA:

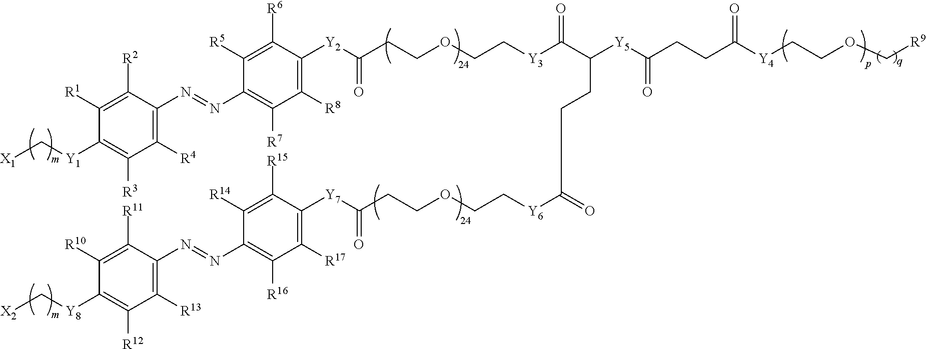

wherein X₁ and X₂ are independently selected from:

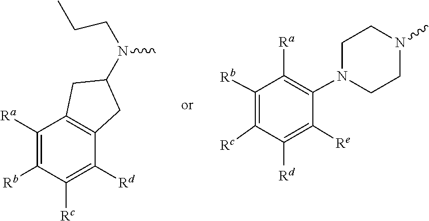

wherein [symbol] represents the X—(C)ₘ— bond;
m is an integer from 1-10;
n is an integer from 1-50;
p is an integer from 1-10;
q is an integer from 1-25;
Y₁, Y₂, Y₃, Y₄, Y₅, Y₆, Y₇ and Y₈ are independently selected from C, N, O or S;
R^{a}, R^{b}, R^{c} and R^{d} are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
R¹, R², R³, R⁴, R⁵, R⁶, R⁷, R⁸, R¹⁰, R¹¹, R¹², R¹³, R¹⁴, R¹⁵, R¹⁶ and R¹⁷ are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
R⁹ is selected from hydrogen, halogen, alkoxy, nitro, amino, hydroxy, cyano, thiol.

Aspect 54. The compound of aspect 53, wherein:
a) one or more of R^{a}, R^{b}, R^{c} and R^{d} is independently selected from F, Cl, Br and I; or
b) one or more of R^{a}, R^{b}, R^{c} and R^{d} is a C(1-6) alkoxy, such as methoxy or ethoxy.

Aspect 55. The compound of aspect 54, wherein the conjugate is a compound of Formula 2A:

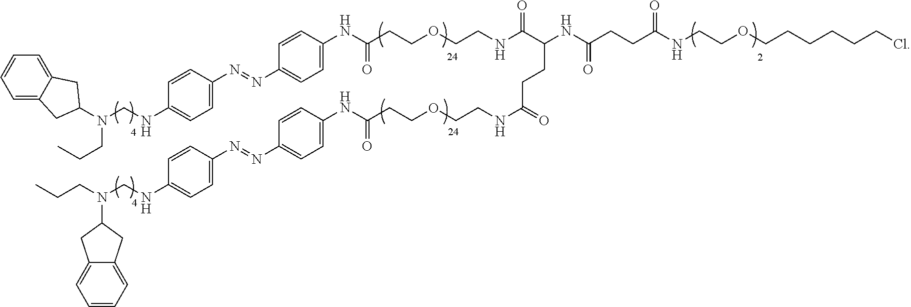

Aspect 56. The compound of aspect 38, wherein the DRL is a D2 dopamine receptor antagonist.

Aspect 57. The compound of aspect 56, wherein the conjugate has the following structure:

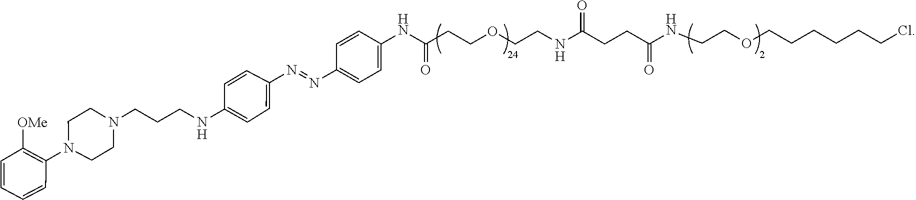

Aspect 58. The compound of aspect 56, wherein the conjugate has the following structure:

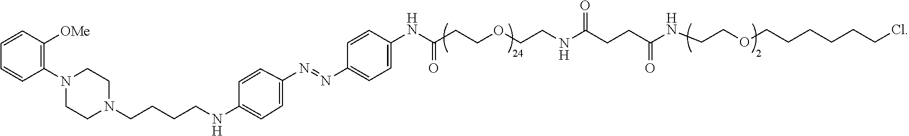

Aspect 59. The compound of aspect 38, wherein the DRL is a D1 dopamine receptor antagonist.

Aspect 60. The compound of aspect 59, wherein the conjugate has the following structure:

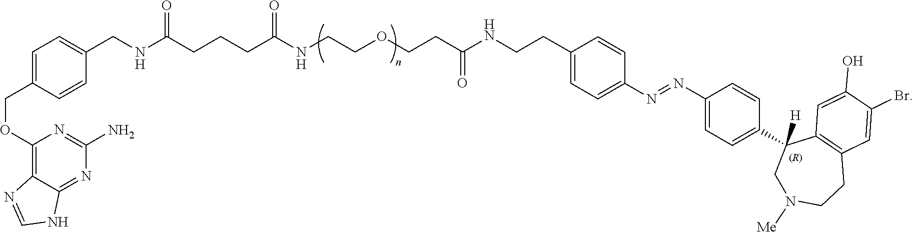

Aspect 61. A method of treating Parkinson's disease in an individual, the method comprising administering the conjugate of any one of aspects 38-60 into the dorsal striatum of the individual.

Aspect 62. The method of aspect 61, comprising exposing the dorsal striatum to light of a wavelength that isomerizes the photoisomerizable agent present in the conjugate.

Aspect 63. The method of aspect 61 or 62, wherein the light is provided by an implantable light source.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Light-Activatable D2 Receptor Agonists

Here, a toolset of Photoswitchable Orthogonal Remotely Tethered Ligands (P) for the D2 family of dopamine receptors is presented. The P is tethered to a HALO-tag membrane anchor protein (M) that is displayed on surface of selected cells to provide optical control of native D2Rs. Importantly, the incorporation of a fast-relaxing azobenzene to different D2R pharmacophores enables the reversible photo-activation or photo-block of D2R, with rapid self-deactivation or unblock in the dark.

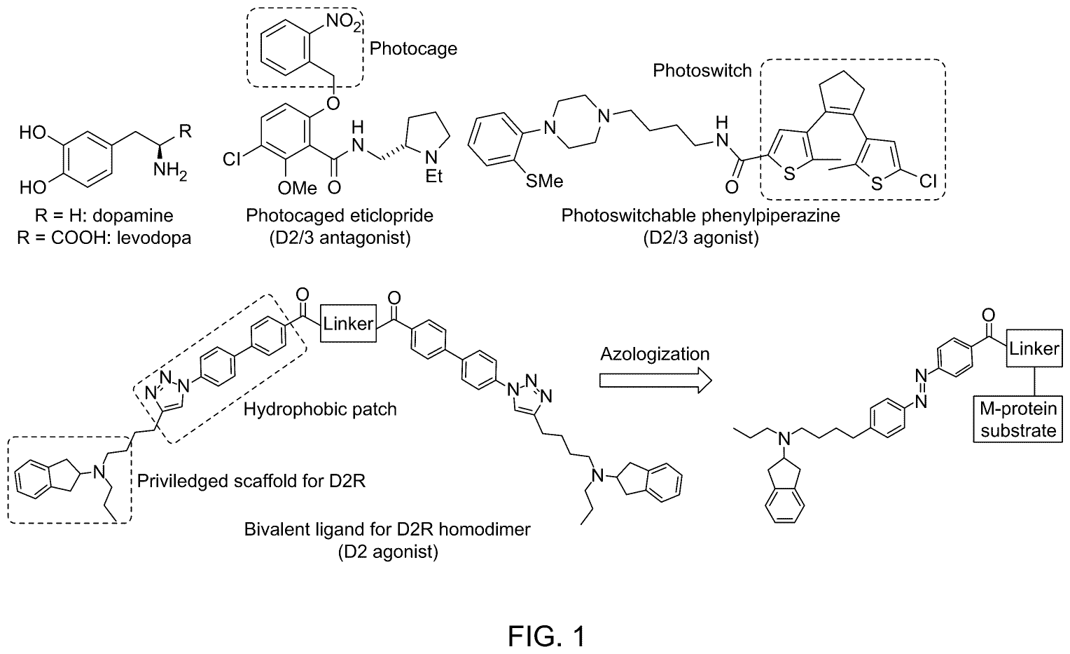

FIG. 1 presents a design of Photoswitchable Orthogonal Remotely Tethered Ligands (P) for D2R.

### Results

The photophysical properties of azobenzenes can be tuned by modifying the substitution pattern on the aromatic core. The search for ideal properties for biological applications has sparked the interest in developing red-shifted fast-relaxing azobenzenes, with higher extinction coefficients and kinetics of switching. The current design of a cis-active D2R agonist is based on a fast-relaxing azobenzene moiety that will allow the usage of only one wavelength of light, along with a rapid, thermal self-deactivation. Phenylpiperazines and 2-aminoindanes are privileged structures for dopamine receptors and mimic the interaction of dopamine in the orthosteric binding pocket. These pharmacophores can be linked via a short, aliphatic linker to a hydrophobic portion, that interacts with a secondary binding site in the D2 receptor. The short aliphatic linker length as well as its substitution pattern can have an influence on subtype selectivity of these constructs, as well as on GPCR functional selectivity. The hydrophobic patch stands out as an ideal unit for an azologization approach and previous work showed this moiety to be amenable to a fulgide or diarylethene photoswitch. Furthermore, it has been shown that this type of construct can be elongated beyond the hydrophobic region with a remote fluorophore or a second ligand for the investigation of GPCR dimerization, hinting that an analogous extension and covalent binding to the M-protein will be tolerated (FIG. 1).

### Synthesis and Functional Analysis of P-D2ago

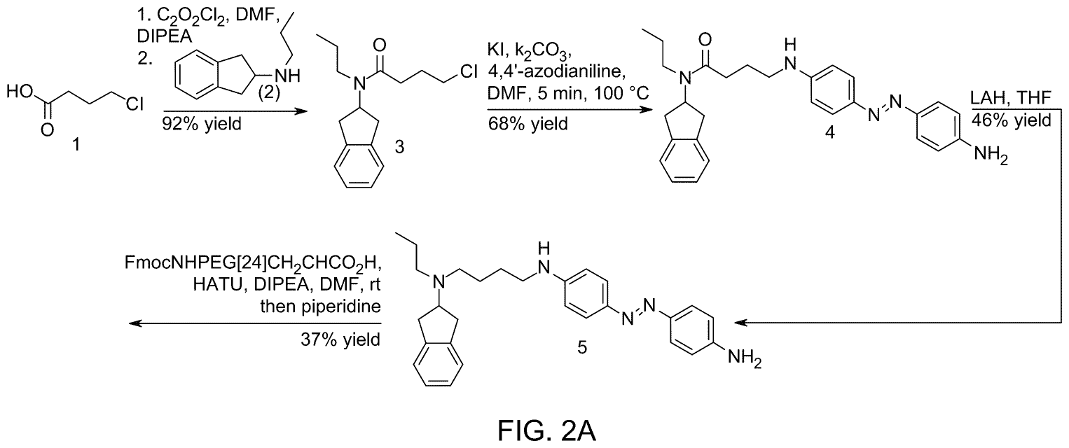

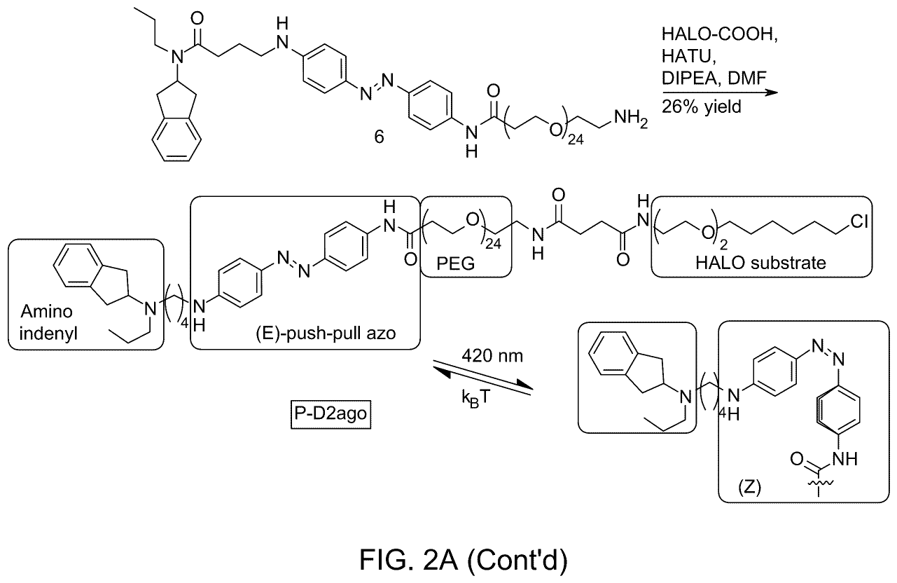

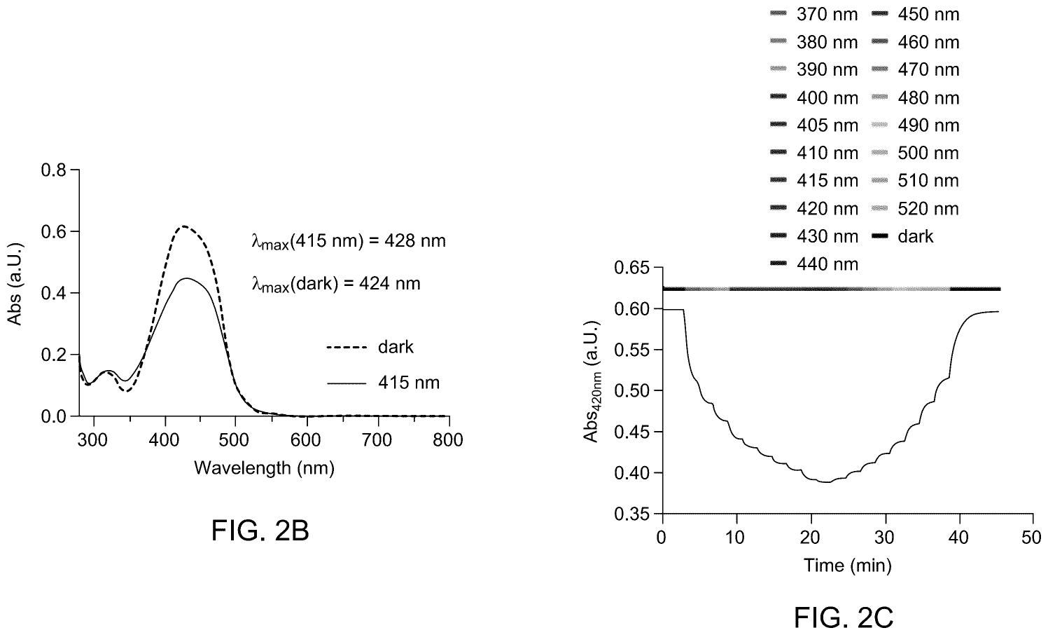

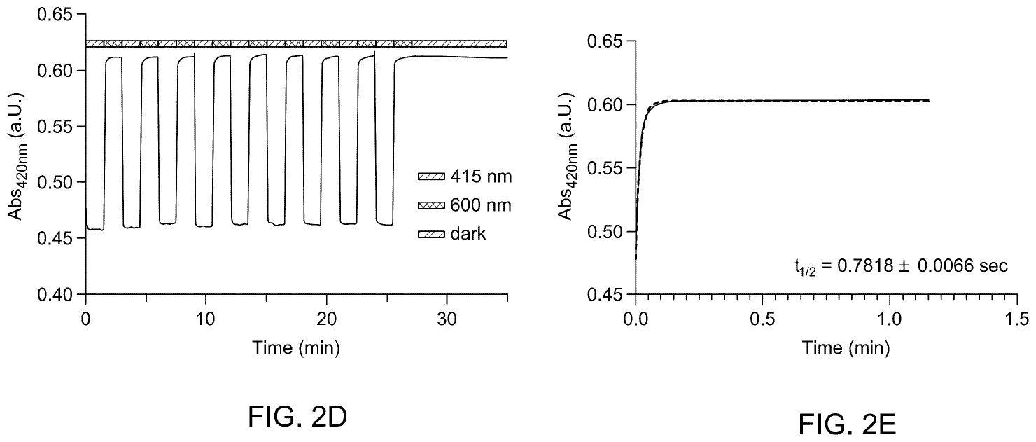

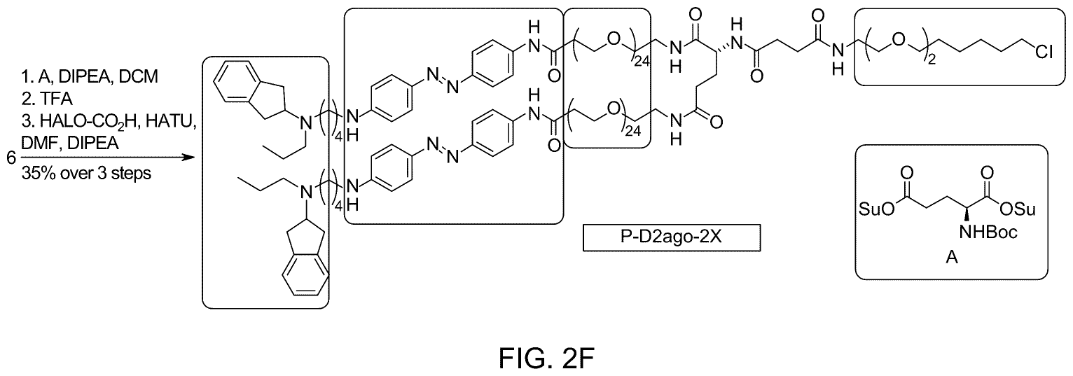

Privileged scaffold 2-aminoindane was chosen as headgroup for the agonist. A 4-carbon chain was installed by acylation of N-propyl 2-aminoindane (2) with freshly prepared acid chloride from acid 1 (FIG. 2A). Upon Finkelstein reaction of 3, the in situ formed primary iodide underwent S_{N}2-reaction with commercial 4,4′-azodianiline. The reduction of disubstituted amide 4 to the tertiary amine 5 proceeded in moderate yields but was tolerated by the azobenzene moiety. A heterobifunctional, carboxylic acid terminated PEG[24]-linker was then installed by HATU coupling with azoaniline 5, followed by unveiling of the primary amine 6 by Fmoc-deprotection. Lastly, HATU coupling with the chloroalkane substrate for the HALO-tag (HALO—COOH) furnished the final construct P-D2ago. Motivated by the recent success of branched PORTLs bearing multiple photoswitchable glutamates in increasing potency and sensitivity, we also synthesized a branched analog of P-D2ago with two azobenzene-2-aminoindanes (P-D2ago-2X, FIG. 2F).¹⁹

UV/Vis studies confirmed the desired switching properties of P-D2ago: The absorption maximum of P-D2ago is 424 nm and maximal switching into the cis-form proceeds at 440 nm (FIG. 2B,C). P-D2ago can be reversibly switched without fatigue (FIG. 2D) and thermally relaxes back into the trans-form with a half-life of 0.78 s in DMSO (FIG. 2E). In aqueous environment (10% DMSO in PBS), the thermal back-relaxation proceeds more rapidly and exceeds the detection limit of the UV/Vis spectrophotometer.

FIG. 2A-2E. Synthesis of P-D2ago and photophysical characterization. A) Chemical Synthesis of P-D2ago. B) UV Vis absorbance spectra of P-D2ago (20 μM in DMSO, 24° C.) in the dark and under 415 nm irradiation. C) Wavelength scan for P-D2ago (20 μM, DMSO, 24° C.). Each wavelength is applied for 2 minutes. A maximum PSS is reached by irradiation with 440 nm. D) Reversible switching of P-D2ago (20 μM, DMSO, 24° C.) by alternating irradiation with 415/600 nm, 90 second irradiations. E) Thermal relaxation of P-D2ago in DMSO after 1 minute irradiation with 415 nm at 37° C. F) Synthesis of branched, photoswitchable amino indenyl dimer P-D2ago-2X.

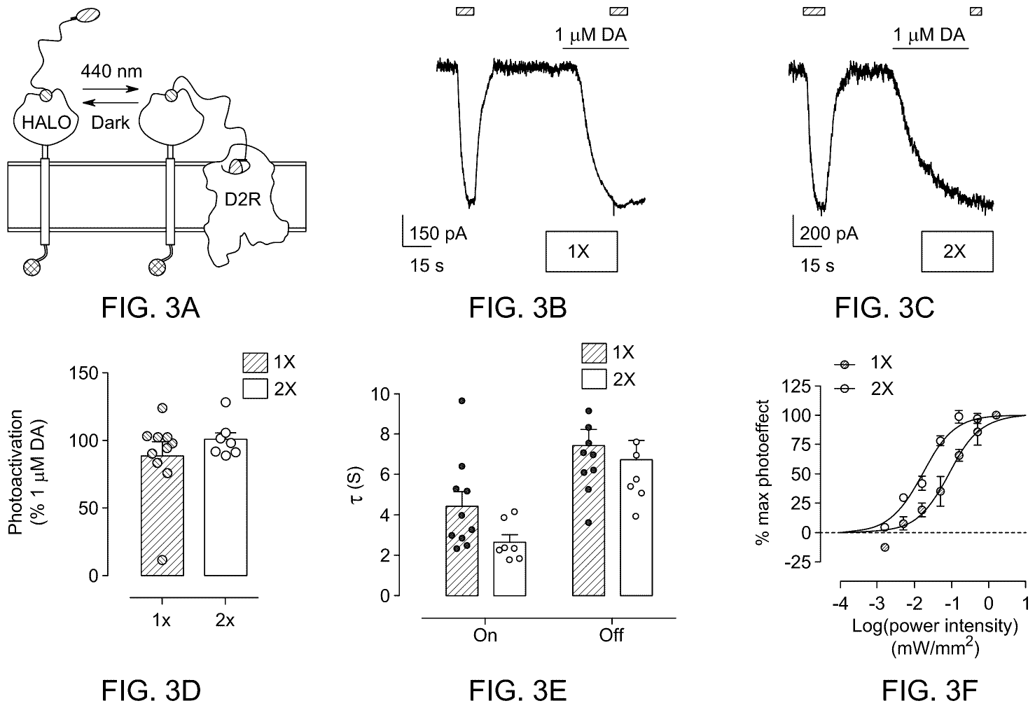

D2R photo-activation was evaluated in a G protein-activated inwardly rectifying K⁺ channel (GIRK) activation assay. The M anchoring protein was re-designed by substituting the SNAP-tag at its external N-terminal with a HALO-tag (FIG. 3A). HEK293T were co-transfected with M, D2R and the homotetramerizing mutant of GIRK1 (F137S), making it possible to detect D2R activation as G_{i/o}-dependent GIRK inward-current at high external K⁺ and negative holding potential. Transfected cells were patch clamped in whole-cell configuration and alternately irradiated with 440 nm light or perfused with a saturating (1 μM) concentration of dopamine to elicit maximal D2R activation. Following incubation in P-D2ago to tether the photoswitch to the M, irradiation with 440 nm light elicited large inward current, with an amplitude similar to current elicited by saturating dopamine (FIG. 3B, D). This demonstrates that P-D2ago achieves full photo-agonism of D2R when tethered to the M. Turning to the branched version of the photoswitch, 2X-P-D2ago, which bears two azobenzene-2-aminoindanes per M anchoring site, we obtained the same maximal activation of D2R (FIG. 3C, D), but faster activation kinetics (˜2-fold) and greater sensitivity to light (˜5-fold) than P-D2ago (FIG. 3E, F).

FIG. 3A-3F: Photoactivation of D2R by MP-D2ago. (A) Schematic representation of D2R activation by MP-D2ago in response to light. (B, C) Representative traces of MP-D2ago with one (1×; B) or two (2×; C) branch points. (D) Summary of the maximal photo-activation of D2R by MP-D2ago relative to a saturating concentration of DA (1 μM). (D, E) Summary of the activation and deactivation kinetics (E) and light sensitivity (F) of D2R in response to 1× or 2×MP-D2ago.

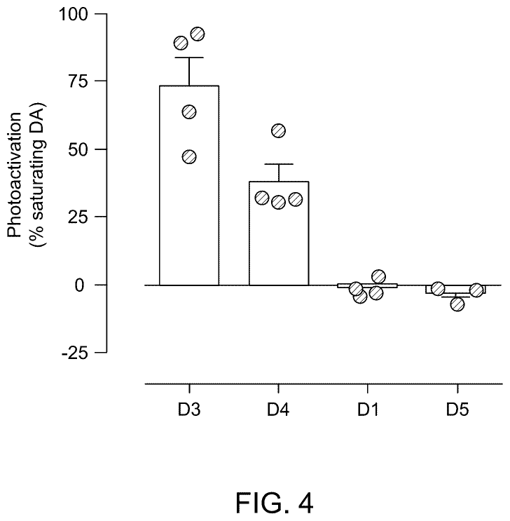

The activity of MP-D2ago containing 2X-P-D2ago was tested on other members within the D2-like receptor subfamily. MP-D2ago was a robust agonist of D3R, the closest homolog of D2R (78% identity; FIG. 4). In contrast, MP-D2ago weakly photo-activated D4R (FIG. 4), which has lower homology with D2R (50% identity). MP-D2ago had no effect on either D1-like receptor, D1R and D5R (FIG. 4). Thus, MP-D2ago is a D2-like receptor agonist.

FIG. 4: Photoactivation of D2-like and D1-like receptors by MP-D2ago. Summary of the maximal photo-activation of D2R by MP-D2ago relative to a saturating concentration of DA (1 μM for D3R and D4R and 10 μM for D1R and D5R).

### Synthesis and Functional Analysis of P-D2antago

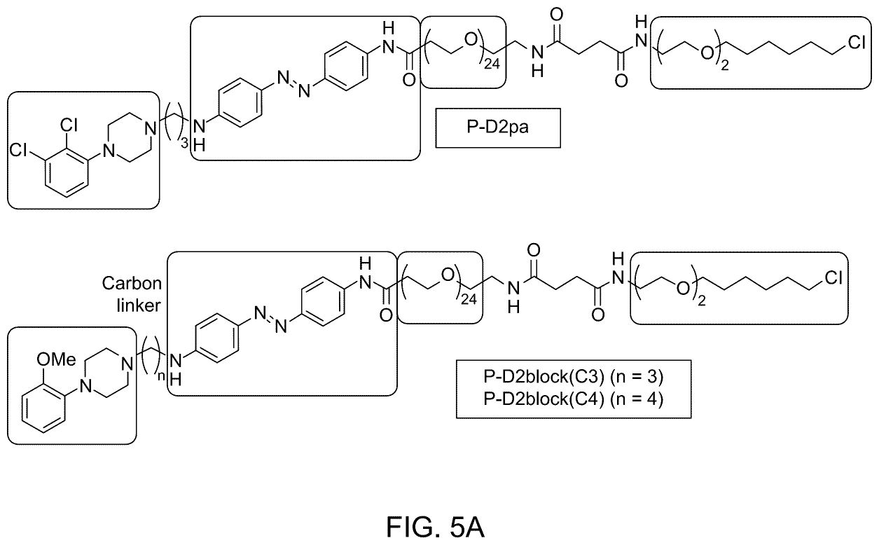

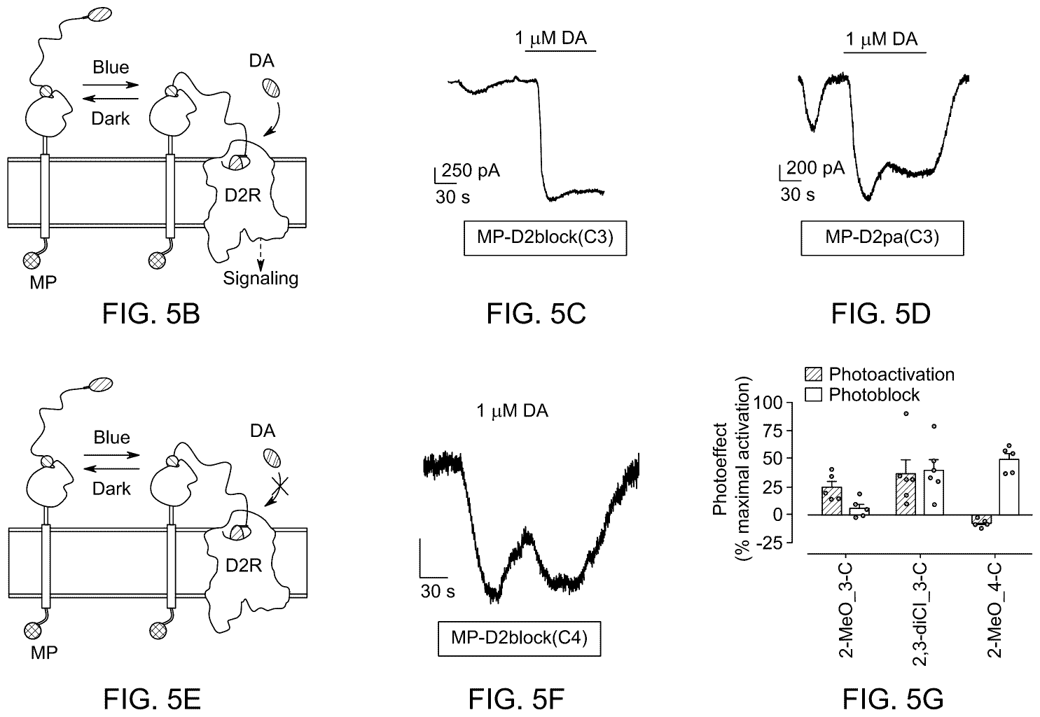

To complement our cell-targeted D2R photo-agonist, a tethered photo-antagonist for D2R was designed. The same photoswitch architecture was employed; however, in this case, one of two substituted phenyl piperazines was used as a head group (FIG. 5A). Two variant phenyl piperazines, 2,3-dichloro- and 2-methoxy-phenylpiperazine, were chosen as the core scaffold. To each of these, we appended a 3-carbon (C3) linker followed by the same PEG- and HALO substrate employed in the synthesis of P-D2ago, thereby generating potential photo-antagonists. After incubating M, D2R and GIRK channel expressing HEK293T cells in each P to covalently attach them to M, we found that both elicit partial photo-agonism. Irradiation with 440 nm light to photo-isomerize from trans to cis elicited very weak partial agonism and very weak antagonism of activation by sub-saturating (1 uM) dopamine with 2-methoxy-phenylpiperazine (FIG. 5C, G). However, the 2,3-dichloro-phenylpiperazine version showed moderate agonism and antagonism of activation by 1 uM dopamine (FIG. 5D, G), indicating that it operates as a partial photo-agonist, which is called P-D2pa(C3). Subtle changes in the aliphatic linker region have been shown to have a dramatic impact on functional selectivity and activity. A 4-carbon linker at this position was tested. It was found that the 2-methoxy-phenylpiperazine P with a 4-carbon linker elicits ˜50% photo-antagonism in the presence of dopamine at 1 uM, and has no photo-agonism on its own (FIG. 5F, G); this compound was called P-D2block (C4).

FIG. 5A-5G: Evaluation of phenylpiperazine-based MPs of D2R in HEK293T cells. A) Chemical structures of P-D2 constructs used to label the M protein. B) Schematic representation MP-D2block,C3 and MP-D2pa,C3 activity under light irradiation. CD) Patch clamp electrophysiology traces of tethered partial agonists. E) Schematic representation of dopamine blocking by MP-D2block under light irradiation. F,G) Patch clamp electrophysiology traces of tethered D2 antagonist.

In summary, a toolkit of photoswitchable ligands has been developed to control D2 receptors in a cell-specific manner. These include a full photo-agonist (P-D2ago), a partial photo-agonist (P-D2pa) and a photo-antagonist (P-D2block(C4)). The ligands are cis-active and show no activity in the thermodynamically more stable trans-configuration. In contrast to bistable azobenzenes that are toggled between the two isomeric states with two different wavelengths of light with photostationary states that yield mixtures of isomers, here, the fast thermal relaxation allows for the complete conversion into the inactive trans-state.

### Example 2: Light-Activatable D1 Receptor Antagonist

Described herein is the synthesis of a light-activatable D1 receptor antagonist.

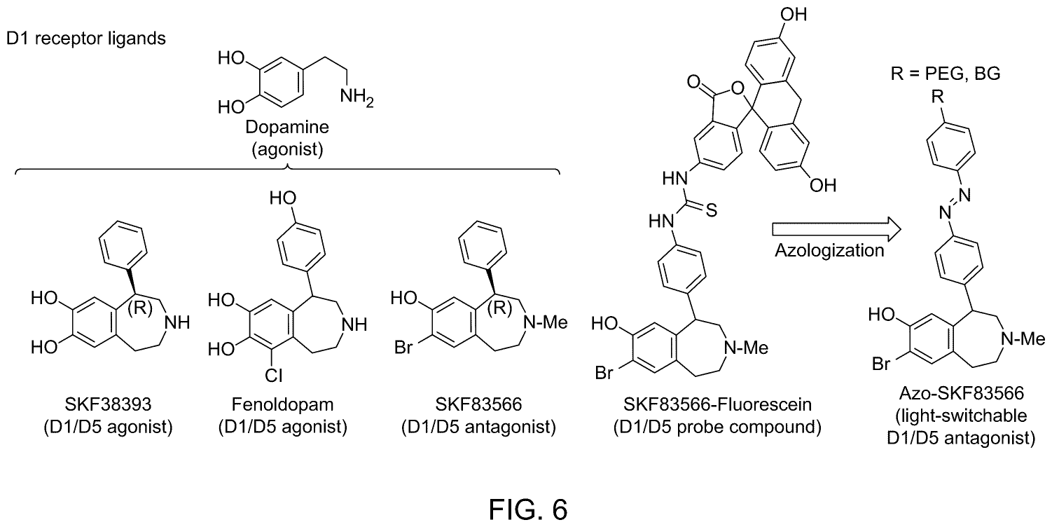

FIG. 6 depicts the chemical structures of D1-like receptor ligands based on the bcnzazcpinc scaffold, a conformationally restricted version of dopamine.

### Materials and Methods

### General Methods, Reagents, Instrumentation

UV Vis data were analyzed and plotted using GRAPHPAD Prism. Irradiation was achieved from the top of the cuvette through a fiber-optic cable. Reversible switching was performed by diluting samples to 20 μM and irradiating with 415/600 nm for 90 seconds each. Absorbance (reported as Abs, in arbitrary units) was measured at 420 nm over time. Thermal Relaxation was measured after 1 min irradiation with 415 nm at 10 or 20 μM, and subsequently absorption increase was detected at 420 nm. The relaxation half-live of the cis-isomer was determined by curve fitting, using exponential one-phase decay in GRAPHPAD Prism. Photostationary states were determined by pre-irradiation of samples (20 uM, DMSO) for 10 min at the respective wavelengths. Samples for LCMS separation were prepared under red-light conditions in amber vials and immediately subjected to LCMS. The relative ratios of (Z)- and (E)-isomers (t_{R}=2.662 min and t_{R}=2.748 min) were determined by detection at the isosbestic point at the respective elution time solvent mixtures (305 nm).

i. 22: 2-(4-nitrophenyl)oxirane

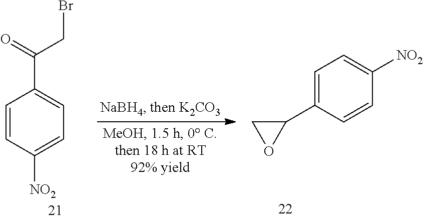 [SMILES: O=C(CBr)c1ccc([N+](=O)[O-])cc1 ; O=[N+]([O-])c1ccc(C2CO2)cc1]

2-(4-nitrophenyl)oxirane was prepared according to the procedure from Vollrath, Benedikt et al from PCT Int. Appl., 201044535, 14 Apr. 2011.

In a 250 mL flame-dried rbf under magnetic stirring, 2-bromo-1-(4-nitrophenyl)ethan-1-one (21) (5.0 g, 20.5 mmol, 1.0 eq) was dissolved in anhydrous MeOH (50 mL, 0.4M) and cooled to 0° C. NaBH₄ (853 mg, 22.5 mmol, 1.10 eq) was added to the reaction mixture at 0° C. in small portions under gas evolution, and the resulting mixture was stirred for 1.5 h until full conversion was determined by TLC. K₂CO₃ (2.8 g, 20.5 mmol, 1.0 eq) was then added at 0° C. and the resulting mixture stirred at room temperature overnight. The reaction mixture was diluted with brine (40 mL) and extracted with diethyl ether (3×100 mL). The combined organic phases were dried over Na₂SO₄ and solvent removed under reduced pressure to yield the product 22 as orange solid in 92% (3.12 g, 18.9 mmol).

R_{f}=0.61 (EtOAc/hexanes, 1:1; UV).

LCMS (5-100% MeCN in H₂O with 0.1% formic acid over 5 min) t_{R}=3.057 min, 230 nm detection.

LRMS (ESI): calc. for C₈H₇NO₃⁺ [M+H]⁺: 166.0; found 166.1.

¹H NMR (400 MHz, CDCl₃): δ=8.2-8.2 (m, 2H), 7.5-7.4 (m, 2H), 4.0 (dd, J=4.1, 2.5 Hz, 1H), 3.2 (dd, J=5.5, 4.1 Hz, 1H), 2.8 (dd, J=5.5, 2.4 Hz, 1H).

¹³C NMR (101 MHz, CDCl₃): δ=148.0, 145.4, 126.4, 124.0, 51.8, 51.6.

IR (neat) 1606 (w), 1519 (s), 1476 (w), 1344 (s), 1316 (w), 1203 (w), 1107 (w), 988 (m), 876 (m), 847 (s), 749 (m) cm⁻¹.

### 23: Amino Alcohol

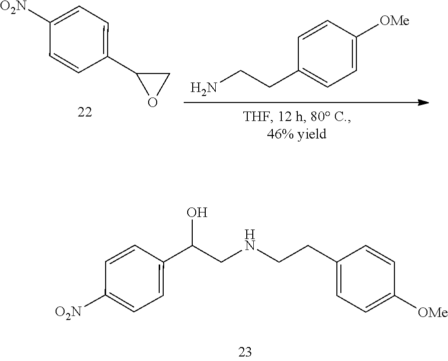

S2 was prepared according to the procedure from J. Med. Chem. 1990, 33, 521-526.

In a 250 mL flame-dried rbf under magnetic stirring, 22 (2.56 g, 15.5 mmol, 1.0 eq) was dissolved in anhydrous THF (25 mL, 0.6M). 2-(4-methoxyphenyl)ethan-1-amine (2.56 g, 16.9 mmol, 1.1 eq) was added to the reaction flask, and the resulting mixture was stirred with a teflon sleeve and a reflux condenser in a metal heating block at reflux (80° C. heating block temperature) for 16 h. The red reaction solution was then cooled to ambient temperature and the solvent was then removed under reduced pressure. The crude red oil was dissolved in 20 mL of Et₂O and cooled to 0° C. for 30 min, and a white solid formed. The precipitate was filtered off, to yield the product 23 as an off-white solid (2.27 g, 7.18 mmol, 46%).

R_{f}=0.36 (5% MeOH in DCM; UV).

LCMS (5-100% MeCN in H₂O with 0.1% formic acid over 5 min) t_{R}=2.647 min, 230 nm detection.

LRMS (ESI): calc. for C₁₇H₂₀N₂O₄⁺ [M+H]⁺: 317.1; found 317.1.

HRMS (ESI): calc. for C₁₇H₂₀N₂O₄⁺ [M+H]⁺: 317.1496; found 317.1498.

¹H NMR (400 MHz, CDCl₃) δ 8.2-8.1 (m, 2H), 7.5 (d, J=8.7 Hz, 2H), 7.2-7.1 (m, 2H), 6.8 (d, J=8.6 Hz, 2H), 4.9 (dd, J=9.5, 3.4 Hz, 1H), 3.8 (s, 3H), 3.1-2.9 (m, 3H), 2.9-2.8 (m, 2H), 2.7 (dd, J=12.3, 9.4 Hz, 1H).

¹³C NMR (101 MHz, CDCl₃): δ=158.4, 149.2, 147.5, 130.4, 129.6, 126.5, 123.7, 114.1, 69.9, 56.1, 55.3, 50.4, 34.4.

IR (neat) 3302 (w), 2896 (w), 2835 (w), 1603 (w), 1509 (s), 1423 (m), 1352 (S), 1240 (s), 1201 (w), 1176 (m), 1107 (s), 1075 (m), 1035 (m), 1014 (w), 986 (w), 906 (m), 884 (m), 856 (m), 837 (m), 818 (s), 753 (m) cm⁻¹.

### 24: Nitro Benzazepine

S3 was prepared according to the procedure from J. Med. Chem. 1990, 33, 521-526.

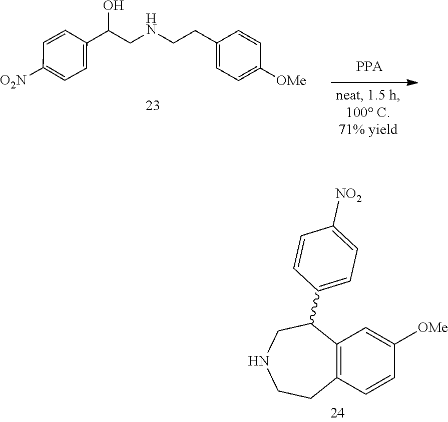 [SMILES: COc1ccc(CCNCC(O)c2ccc([N+](=O)[O-])cc2)cc1 ; COc1ccc2c(c1)C(c1ccc([N+](=O)[O-])cc1)CNCC2]

In a 50 mL rbf under magnetic stirring, 23 (1.80 g, 5.69 mmol, 1.0 eq) was combined with polyphosphoric acid (115%, 7.9 mL, 0.7M), heated to 100° C. in an oil bath, and stirred for 2 h. The dark brown reaction mixture was poured while hot into an Erlenmeyer flask with ice-water (10 mL) to form a brown mixture, and then NH₄OH (20 mL) was added to bring the mixture pH above 9. At pH >9, the product turned red and was extracted with DCM (2×100 mL), and the combined organic layers dried over Na₂SO₄ and concentrated under reduced pressure. The product 24 was recovered as a red solid (1.20 g, 4.02 mmol, 71%) and used without further purification. An analytical sample was purified on normal phase semi prep (0-5% MeOH in DCM over 20 min, 19 mL/min, 20 mm×250 mm×5 uM) to yield a pure sample of 24 for characterization.

R_{f}=0.28 (5% MeOH in DCM; UV).

LCMS (5-100% MeCN in H₂O with 0.1% formic acid over 5 min) t_{R}=2.510 min, 360 nm detection.

LRMS (ESI): calc. for C₁₇H₁₈N₂O₃⁺ [M+H]⁺: 299.1; found 299.1.

HRMS (ESI): calc. for C₁₇H₁₈N₂O₃⁺ [M+H]⁺: 299.1390; found 299.1395.

¹H NMR (400 MHz, CDCl₃) δ 8.2 (d, J=8.4 Hz, 2H), 7.3 (d, J=8.4 Hz, 2H), 7.1 (d, J=8.3 Hz, 1H), 6.7 (dd, J=8.3, 2.7 Hz, 1H), 6.4 (dd, J=4.8, 2.7 Hz, 1H), 4.4 (d, J=6.8 Hz, 1H), 3.7-3.6 (m, 4H), 3.5-3.4 (m, 1H), 3.1-2.7 (m, 4H).

¹³C NMR (101 MHz, CDCl₃) δ 158.4, 149.4, 146.7, 143.1, 143.1, 133.2, 131.7, 129.3, 124.0, 116.4, 116.4, 111.2, 55.3, 52.5, 52.4, 48.2, 37.9.

IR (neat)) v=2924 (w), 2835 (w), 1607 (m), 1518 (s), 1462 (w), 1347 (s), 1269 (w), 1108 (w), 1049 (w), 856 (w) cm⁻¹.

### i. 25: N-Methyl Benzazepine

25 was prepared according to the procedure from J. Med. Chem. 1990, 33, 521-526.

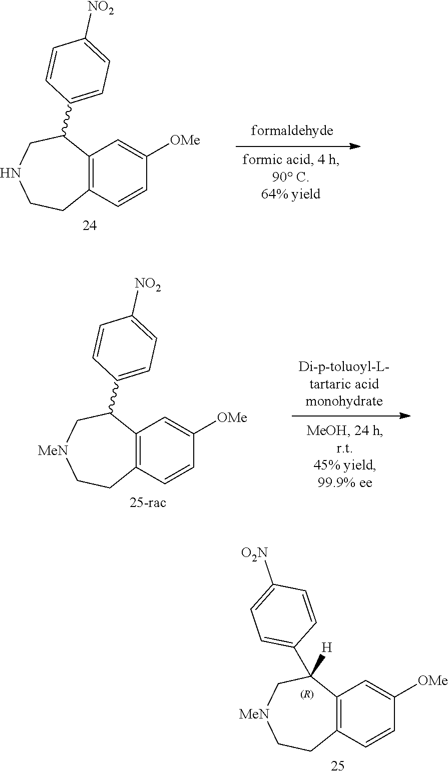

In a 50 mL rbf, 25 (2.60 g, 8.72 mmol, 1.00 eq) was dissolved in formic acid (44 mL) and connected to a nitrogen line via a needle and rubber septum. Then, under stirring, formaldehyde (37% solution in water, stabilized with MeOH; 6.48 mL, 79.8 mmol, 9.16 eq) was added dropwise. The rubber septum was replaced with a Teflon sleeve and a reflux condenser, connected to a nitrogen line. The reaction mixture was heated to 90° C. in an oil-bath for 4 h. The reaction mixture was cooled to room temperature, and added dropwise to a 500 mL Erlenmeyer flask containing ice-water (100 mL) and an ammonia solution (aq., 2M, 100 mL) under strong formation of fumes. The white precipitate forming dissolves upon addition of DCM (50 mL). The aq. phase was separated and extracted three times with DCM (3×50 mL). The combined organic phases were dried over Na₂SO₄, then concentrated in vacuo. The racemic product 25-rac was obtained as a light orange oil in 64% yield (1.75 g, 5.61 mmol).

### Chiral Resolution:

The racemic product 25-rac (2.18 g, 6.98 mmol, 1.00 eq) was dissolved in MeOH (10 mL) in a 100 mL round bottom flask and di-p-toluoyl-L-tartaric acid monohydrate (2.84 g, 7.02 mmol, 1.00 eq) was added. The mixture was warmed in a metal heat block with a reflux condenser under magnetic stirring to 50° C., until all solid dissolved. The mixture was let stand at ambient temperature for 24 h, until white solid formed, which was harvested over a filter funnel under reduced pressure and washed with little amounts of Et₂O. The desired salt was obtained (2.60 g) and recrystallized from MeOH. The obtained salt after recrystallization (1.51 g) was dissolved in water (30 mL) and NaHCO₃ (sat., aq., 70 mL). The aqueous phase was extracted three times with EtOAc (50 mL), and the combined, yellow organic phases were washed with brine (20 mL) and dried over Na₂SO₄. The organic phase was filtered and concentrated under reduced pressure to yield a red oil, that was subjected to the same procedure. After two rounds of crystallization, the R-enantiomer 25 was obtained as a red oil in 30% yield (0.66 g, 2.1 mmol). The combined filtrates containing the undesired S-enantiomer were treated identically by free-basing with sodium bicarbonate, to yield 70% of the product enriched in (S)-isomer (1.52 g, 4.86 mmol).

Enantiomeric excess of the obtained desired R-(+)-enantiomer was determined by HPLC analysis on chiral stationary phase (CHIRALCEL OD-H, 4.6×250 mm, 24° C., 0.8 mL/min, 4% i-PrOH in n-heptane, detection at 280 nm) to be 99.5% by comparison with a racemic sample; t_{R} (R-(+)-enantiomer)=11.702 min, t_{R} (S-(−)-enantiomer)=14.410 min. The obtained (S)-enantiomer in the filtrate had low enantiomeric purity and was treated in the same fashion with di-p-toluoyl-D-tartaric acid monohydrate to yield the pure (S)-enantiomer.

R_{f}=0.21 (5% MeOH in DCM; UV).

LCMS (5-100% MeCN in H₂O with 0.1% formic acid over 5 min) t_{R}=2.605 min, 360 nm detection.

LRMS (ESI): calc. for C₁₈H₂₁N₂O₃⁺ [M+H]⁺: 313.2; found 313.1.

HRMS (APCI): calc. for C₁₈H₂₁N₂O₃⁺ [M+H]⁺: 313.1547; found 313.1555.

¹H NMR (400 MHz, CDCl₃) δ 8.2 (d, J=8.7 Hz, 2H), 7.4-7.3 (m, 2H), 7.1 (d, J=8.2 Hz, 11H), 6.7 (dd, J=8.2, 2.7 Hz, 11H), 6.3 (d, J=2.7 Hz, 11H), 4.3 (d, J=7.4 Hz, 11H), 3.7 (s, 3H), 3.2-3.1 (m, 1H), 3.0-2.8 (m, 2H), 2.8-2.6 (m, 2H), 2.6-2.5 (m, 1H), 2.4 (s, 3H).

¹³C NMR (101 MHz, CDCl₃): δ=158.2, 150.5, 146.4, 143.7, 133.5, 131.0, 129.2, 123.7, 115.6, 110.9, 61.8, 57.7, 55.2, 50.4, 48.0, 35.4.

For (R)-enantiomer: [α]_{D}=+45.5 (c=0.5 in MeOH).

For (S)-enantiomer: [α]_{D}=−43.4 (c=0.5 in MeOH).

IR (neat) 2937 (w), 2837 (w), 2797 (w), 1652 (w), 1604 (m), 1580 (w), 1517 (s), 1462 (m), 1384 (w), 1346 (s), 1265 (w), 1159 (w), 1108 (m), 1040 (w), 1014 (w), 856 (m), 842 (w), 815 (w), 740 (w) cm⁻¹.

### i. 26: R-Bromo Nitro Benzazepine

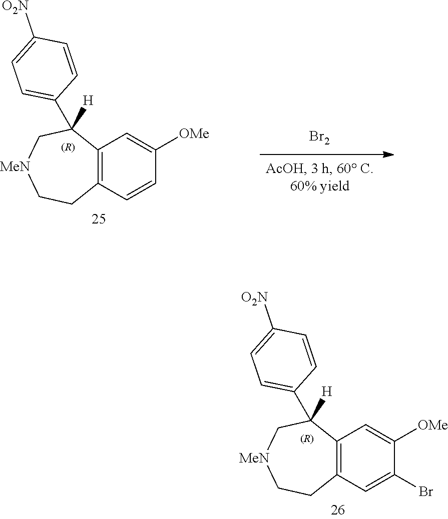

26 was prepared according to the procedure from J. Med. Chem. 1990, 33, 521-526.

In a 100 mL three-neck round bottom flask, 25 (0.500 g, 1.60 mmol, 1.00 eq) was dissolved in AcOH (35 mL) and warmed to 60° C. with a reflux condenser and a 250 mL dropping funnel, attached to a Schlenck line under N₂ pressure. Bromine (0.460 g, 3.20 mmol, in 10 mL AcOH) was transferred to the dropping funnel and added dropwise at 60° C. The red solution formed an abundant white precipitate upon addition, which re-dissolved under stirring. After 3 h, the solution was cooled to ambient temperature, poured onto ice-water (50 mL) and treated with ammonium hydroxide (10% aq., 50 mL) and the mixture was extracted with DCM (30 mL×3). The combined yellow organic phases were washed with brine (20 mL) and dried over Na₂SO₄. The solution was filtered and concentrated under reduced pressure, to yield the crude product as an off-yellow foam. The crude product was subjected to flash column chromatography (SiO₂, 24 g, 0-100% EtOAc in DCM) and the purified product 26 was collected as a white foam in 60% yield (377 mg, 0.964 mmol).

R_{f}=0.59 (5% MeOH in DCM; UV).

LCMS (5-100% MeCN in H₂O with 0.1% formic acid over 5 min) t_{R}=3.011 min, 254 nm detection.

LRMS (ESI): calc. for C₁₈H₂₀BrN₂O₄⁺ [M+H]⁺: 391.1; found 391.0.

HRMS (APCI): calc. for C₁₈H₂₀BrN₂O₄⁺ [M+H]⁺: 391.0652; found 391.0654.

¹H NMR (400 MHz, CDCl₃) δ 8.2-8.1 (m, 2H), 7.4-7.3 (m, 3H), 6.3 (s, 1H), 4.4 (d, J=7.3 Hz, 1H), 3.7 (s, 3H), 3.2-3.1 (m, 1H), 2.9-2.8 (m, 2H), 2.8-2.7 (m, 1H), 2.7-2.5 (m, 2H), 2.4 (s, 3H).

¹³C NMR (101 MHz, CDCl₃) δ 154.4, 149.9, 146.6, 142.8, 134.9, 134.7, 129.1, 123.8, 113.3, 109.6, 61.4, 57.3, 56.3, 50.3, 47.9, 34.9.

IR (neat) 3399 (b), 2929 (m), 2854 (w), 1598 (m), 1519 (s), 1495 (m), 1463 (w), 1383 (w), 1346 (s), 1271 (w), 1253 (w), 1110 (w), 1053 (w), 1015 (w), 856 (m) cm⁻¹.

For (R)-enantiomer: [α]_{D}=+17.2 (c=0.5 in MeOH).

### i. 27: R-Bromo Benzazepine Aniline

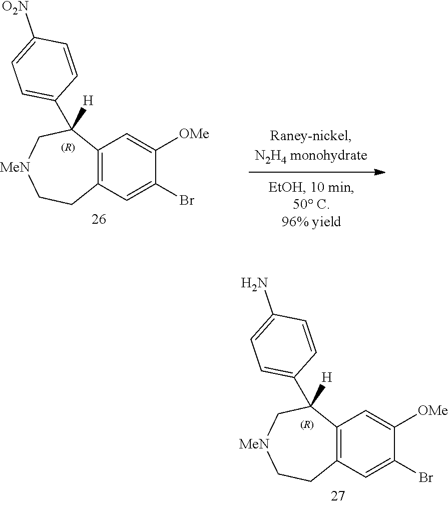

27 was prepared according to the procedure from J. Med. Chem. 1990, 33, 521-526.

In a 25 mL round bottom flask, 26 (0.110 g, 0.284 mmol, 1.00 eq) was dissolved in EtOH (5.6 mL), and hydrazine monohydrate (58 μL, 1.19 mmol, 4.20 eq) were added. The reaction mixture was warmed to 50° C. in an oil bath, and Raney Nickel was added as a suspension in water dropwise, until bubbling ceased after about 10 min. The reaction mixture was filtered over celite to remove the Nickel and the crude mixture was concentrated under reduced pressure. The crude yellow oil was subjected to flash column chromatography (SiO₂, 4 g, 0-5% MeOH in DCM) to yield the product 27 as light yellow oil in 96% (98 mg, 0.27 mmol).

R_{f}=0.16 (5% MeOH in DCM; UV).

LCMS (5-100% MeCN in H₂O with 0.1% formic acid over 5 min) t_{R}=1.978 min, 254 nm detection.

LRMS (ESI): calc. for C₁₈H₂₁BrN₂O [M+H]⁺: 361.1; found 361.0.

HRMS (APCI): calc for C₁₈H₂₁BrN₂O [M+H]⁺: 361.0910; found 361.0905.

¹H NMR (400 MHz, CDCl₃) δ 7.3-7.3 (m, 1H), 7.0-6.9 (m, 2H), 6.7-6.6 (m, 2H), 6.3 (s, 1H), 4.2 (d, J=8.5 Hz, 1H), 3.6 (s, 3H), 3.1-2.9 (m, 2H), 2.9-2.7 (m, 3H), 2.4-2.3 (m, 4H).

¹³C NMR (101 MHz, CDCl₃): δ=154.1, 145.8, 145.0, 134.9, 133.8, 132.7, 129.2, 115.5, 113.0, 108.4, 63.1, 57.3, 56.2, 49.2, 47.8, 35.2.

For (R)-enantiomer: [α]_{D}=+28.4 (c=0.5 in MeOH).

IR (neat) 3353 (b), 2937 (w), 2846 (w) 2797 (w), 1622 (m), 1516 (s), 1491 (s), 1460 (m), 1379 (m), 1270 (m), 1249 (m), 1181 (m), 1076 (w), 1051 (m), 943 (w), 836 (m), 742 (m), 707 (m) cm⁻¹.

### i. 28: Azo-Benzazepine

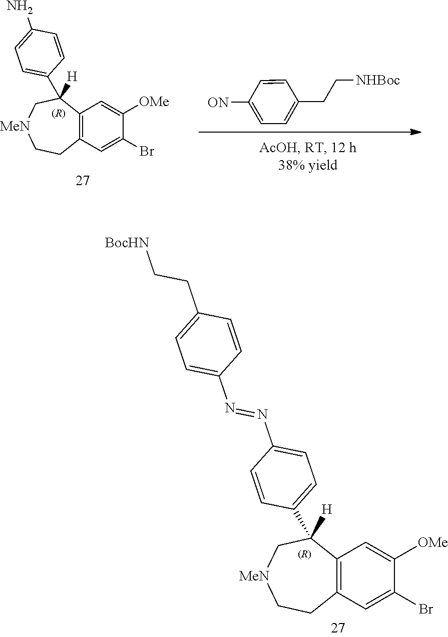

In a 150 mL round bottom flask, tert-butyl (4-aminophenethyl)carbamate (0.181 g, 0.767 mmol, 1.50 eq) was dissolved in DCM (13 mL). Oxone (1.57 g, 2.55 mmol, 5.00 eq) and water (13 mL) were added, and the biphasic mixture was vigorously stirred for 3 h. The green organic layer was separated off, and the aqueous phase was extracted with DCM (20 mL×2). The combined organic layers were washed with bicarbonate (sat., aq., 10 mL) and dried over Na₂SO₄. The organic phase was filtered and concentrated under reduced pressure to an around 3 mL, green solution containing the intermediate nitroso benzene. In a separate 50 mL round bottom flask, 27 (185 mg, 0.511 mmol, 1.00 eq) in AcOH (7.7 mL) and the prepared nitrosobenzene was added as a solution in DCM. The DCM was evaporated at 500 mbar on the rotary evaporator and the reaction mixture stirred for 12 h. The reaction mixture was concentrated on the rotary evaporator and the crude oil subjected to column chromatography (SiO₂, 24 g, 0-10% MeOH in DCM) to yield the product 28 as yellow oil in 38% yield (115 mg, 0.195 mmol).

R_{f}=0.16 (1:1 EA:hexanes; Vis, yellow spot).

LCMS (5-100% MeCN in H₂O with 0.1% formic acid over 5 min) t_{R}=3.863 min, 360 nm detection.

LRMS (ESI): calc. for C₃₁H₃₈BrN₄O₃⁺ [M+H]⁺: 593.2122; found 593.2.

HRMS (APCI): calc. for C₃₁H₃₈BrN₄O₃⁺ [M+H]⁺: 595.2101; found 595.2100.

¹H NMR (400 MHz, CDCl₃) δ 7.9-7.8 (m, 4H), 7.4-7.3 (m, 5H), 6.3 (s, 1H), 4.4 (d, J=8.1 Hz, 1H), 3.6 (s, 3H), 3.4-3.3 (m, 2H), 3.2-2.9 (m, 3H), 2.9-2.7 (m, 4H), 2.4-2.3 (m, 4H), 1.4 (s, 9H).

¹³C NMR (101 MHz, CDCl₃) δ 155.9, 154.1, 151.3, 151.3, 145.5, 144.2, 142.5, 134.7, 134.0, 129.5, 129.0, 123.1, 123.0, 112.8, 108.8, 79.2, 62.1, 56.9, 56.0, 49.4, 47.4, 41.6, 36.1, 34.6, 28.4.

IR (neat) 3331 (b), 2938 (m), 2798 (m), 1598 (m), 1515 (s), 1494 (m), 1461 (w), 1384 (w), 1343 (s), 1251 (m), 1077 (w), 1032 (m), 921 (w), 855 (m), 843 (w), 815 (w), 714 (m) cm⁻¹.

### 29: Phenol-Benzazepine Azo

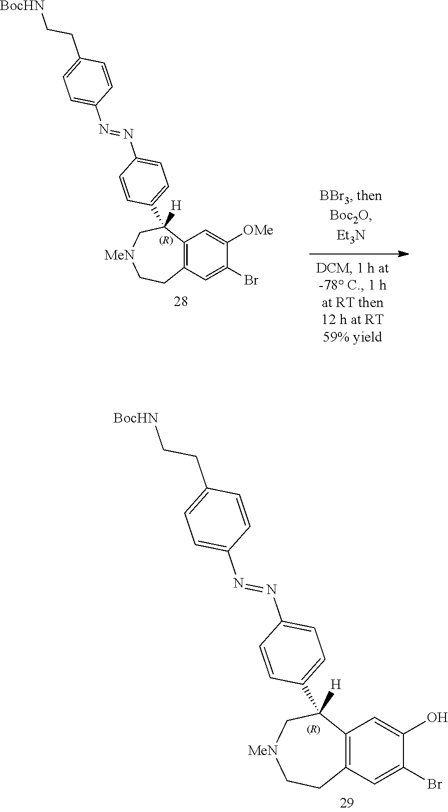

In a flame-dried 100 mL round bottom flask under magnetic stirring, 28 (90.8 mg, 0.153 mmol, 1.00 eq) was dissolved in DCM (22 mL). The solution was cooled to −78° C. in a dry ice/acetone bath. In a separate flame-dried 10 mL round bottom flask, BBr₃ (72 μL, 0.765 mmol, 5.00 eq) was dissolved in hexane (3.3 mL). The BBr₃-solution was added dropwise to the reaction flask at −78° C. and stirred for 1 h. The reaction was then allowed to warm to ambient temperature and continued to stir for 1 h. After this time, the reaction was cooled back to −78° C. and MeOH (75 mL) was slowly added to quench remaining BBr₃. The mixture was concentrated under reduced pressure, and the crude material co-evaporated with MeOH (2×10 mL) to remove any residual B(OCH₃)₃. To re-protect any deprotected amine, the crude oil was dissolved in DCM (1.5 mL) and Et₃N (213 μL, 1.53 mmol, 10.0 eq) and Boc₂O (40.1 mg, 0.184 mmol, 1.20 eq) were added to the solution. This brown suspension was stirred at ambient temperature for 12 h. The reaction mixture was treated with NaHCO₃ (aq., 5 mL) and extracted with DCM (3×15 mL). The combined organic phases were dried with Na₂SO₄, filtered, and concentrated under reduced pressure to yield a crude oil. The crude product was purified via flash column chromatography (SiO₂, 12 g, 0-10% MeOH in DCM) and the product 29 was obtained as an orange oil in 59% yield (52.0 mg, 0.090 mmol).

R_{f}=0.34 (5% MeOH and 15% DCM in EtOAc; UV; yellow spot).

LCMS (5-100% MeCN in H₂O with 0.1% formic acid over 5 min) t_{R}=3.718 min, 254 nm detection.

LRMS (ESI): calc. for C₃₀H₃₆BrN₄O₃⁺ [M+H]⁺: 579.2; found 579.2.

HRMS (APCI): calc. for C₃₀H₃₆BrN₄O₃⁺ [M+H]⁺: 581.1945; found 581.1949.

IR (neat) 3405 (b), 2916 (w), 1761 (w), 1701 (m), 1600 (m), 1498 (m), 1434 (m), 1405 (m), 1365 (m), 1341 (w), 1309 (w), 1246 (m), 1162 (m), 1014 (S), 950 (m), 853 (w) cm⁻¹.

¹H NMR (400 MHz, CDCl₃) δ 7.9-7.8 (m, 4H), 7.3 (d, J=8.2 Hz, 2H), 7.2 (s, 1H), 7.2 (d, J=8.2 Hz, 2H), 6.2 (s, 1H), 4.2 (d, J=8.8 Hz, 1H), 3.5-3.4 (m, 2H), 3.1-3.0 (m, 2H), 3.0-2.8 (m, 4H), 2.7-2.7 (m, 1H), 2.4-2.2 (m, 4H), 1.4 (s, 9H).

¹³C NMR (101 MHz, CDCl₃) δ 156.0, 151.5, 151.5, 151.4, 145.7, 145.1, 142.6, 134.2, 133.0, 129.7, 129.6, 129.3, 129.2, 129.2, 123.2, 123.2, 116.5, 107.5, 79.5, 62.6, 57.2, 48.9, 47.3, 41.7, 36.2, 34.8, 28.5.

### i. 30: Azo-Peg[12]

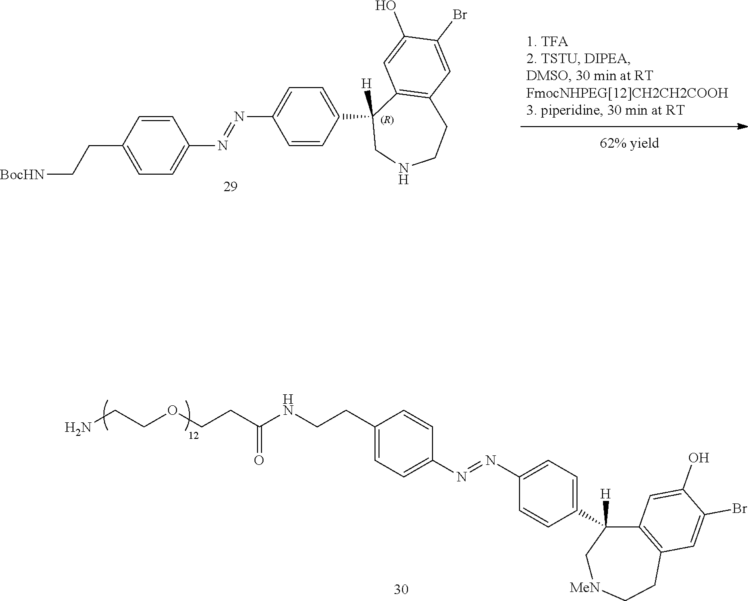

In a 1.5 mL vial under ambient conditions, FmocNHPEG[12]CH₂CH₂COOH (32.1 mg, 0.038 mmol, 2.7 eq) was dissolved in DMSO (109 uL). TSTU (11.5 mg, 0.038 mmol, 2.7 eq) and DIPEA (20 uL, 0.115 mmol, 8.1 eq) were added. The reaction was stirred for 30 min until full conversion was determined by LCMS analysis. In the meanwhile, in a 1.5 mL vial, 29 (8.2 mg, 0.014 mmol, 1.0 eq) was Boc-deprotected by treatment with TFA (100 uL). The TFA was removed after 30 seconds by evaporation under a nitrogen stream. The crude azo amine was dissolved in DMSO (80 uL) and added to the activated acid with an additional amount of DIPEA (5 uL, 0.028 mmol, 2.0 eq). The reaction was stirred for 1 h, until full conversion was determined by LCMS. Piperidine (140 uL, 1.415 mmol, 100 eq) was added to the reaction mixture and stirring was continued until full conversion was determined by LCMS. The crude reaction mixture was cooled in an ice-water bath and treated with HOAc (97 uL, 1.698 mmol, 120 eq). The mixture was purified by RP-HPLC (15-35% MeCN in H₂O with 0.1% formic acid over 5 min, semi-preparative column, 9.0 mL/min flowrate, detection at 360 nm, t_{R}=2.791 min) to yield the product 30 after lyophilization as yellow oil in 62% yield (9.4 mg, 0.009 mmol).

LCMS (5-100% MeCN in H₂O with 0.1% formic acid over 5 min) t_{R}=2.791 min, 360 nm detection.

LRMS (ESI): calc. for C₅₂H₈₂BrN₅O₁₄²⁺ [M+2H]²⁺: 539.75; found 539.8.

HRMS (APCI): calc. for C₅₂H₈₁BrN₅O₁₄⁺ [M+H]⁺: 1080.4937; found 1080.4944.

¹H NMR (400 MHz, MeOD) δ 8.0 (d, J=8.2 Hz, 2H), 7.9 (d, J=8.2 Hz, 2H), 7.5-7.4 (m, 4H), 7.3 (s, 1H), 6.3 (s, 1H), 4.5 (d, J=8.6 Hz, 1H), 3.8-3.8 (m, 3H), 3.7-3.6 (m, 43H), 3.5 (t, J=7.1 Hz, 3H), 3.3 (s, 1H), 3.2-3.1 (m, 5H), 3.0-2.8 (m, 3H), 2.7 (t, J=6.4 Hz, 1H), 2.5 (s, 4H), 2.4 (t, J=6.0 Hz, 2H).

¹³C NMR (101 MHz, MeOD) δ 172.6, 152.5, 151.5, 151.3, 145.4, 143.9, 143.2, 133.4, 132.8, 129.5, 129.1, 129.0, 122.9, 122.6, 116.1, 106.7, 70.1, 70.1, 70.1, 70.0, 70.0, 70.0, 70.0, 69.9, 69.9, 69.9, 69.8, 69.8, 69.8, 69.8, 69.7, 69.7, 69.7, 69.6, 69.5, 69.3, 69.3, 67.1, 66.9, 66.6, 66.5, 61.9, 56.9, 42.6, 40.2, 39.3, 39.3, 34.9, 33.2, 33.0.

i. P-D1block(12)

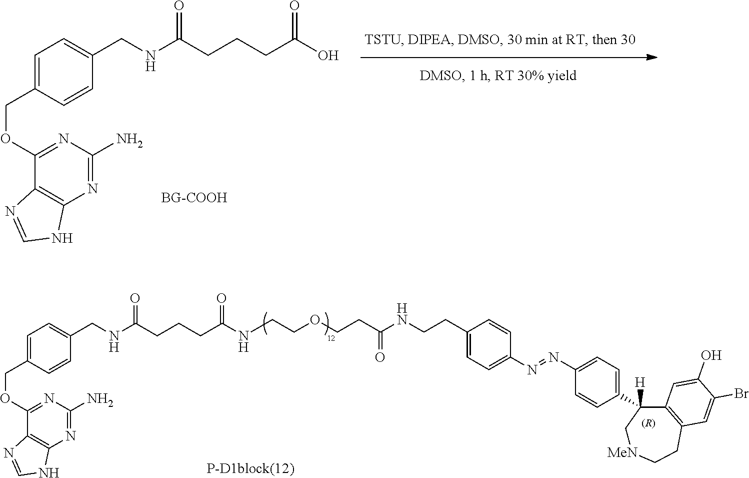

In a 1.5 mL vial, BG-COOH (2.7 mg, 0.007 mmol, 1.5 eq) was dissolved in DMSO (36 uL). TSTU (2.1 mg, 0.007 mmol, 1.5 eq) and DIPEA (3 uL, 0.019 mmol, 4.0 eq) were added and the reaction mixture was stirred for 30 min, until full conversion was determined by LCMS analysis. 30 (5.0 mg, 0.005 mmol, 1.0 eq) was added to the reaction mixture as a solution in DMSO (26 uL) and more DIPEA (3 uL, 0.019 mmol, 4.0 eq) was added. After the reaction was judged complete by LCMS analysis, the mixture was purified by RP-HPLC (10-45% MeCN in H₂O with 0.1% formic acid over 9 min, semi-preparative column, 9.0 mL/min flow rate, detection at 360 nm, t_{R}=6.397 min) to yield the product after evaporation of the solvent at 40° C. in vacuo as a yellow oil in 30% yield (2.0 mg, 0.001 mmol). P-D1block(12) was synthesized using starting materials of >99% ee. Furthermore, a P-D1block(12,S) version was synthesized using S-configured starting materials of 56% ee.

LCMS (5-100% MeCN in H₂O with 0.1% formic acid over 5 min) t_{R}=2.826 min, 360 nm detection.

LRMS (ESI): calc. for C₇₀H₁₀₀BrNi₁₁O₁₇²⁺ [M+2H]²⁺: 722.8236; found 722.8.

HRMS (ESI): calc. for C₇₀H₁₀₀BrN₁₁O₁₇²⁺ [M+2H]²⁺: 722.8236; found 722.8223.

¹H NMR (400 MHz, MeOD) δ 8.0 (d, J=8.3 Hz, 2H), 7.9-7.8 (m, 3H), 7.5 (d, J=7.9 Hz, 2H), 7.4 (t, J=8.6 Hz, 4H), 7.4 (s, 1H), 7.3 (d, J=7.8 Hz, 2H), 6.3 (s, 1H), 5.5 (s, 2H), 4.5 (d, J=8.8 Hz, 1H), 4.4 (s, 2H), 3.7 (t, J=6.0 Hz, 2H), 3.6-3.6 (m, 47H), 3.5-3.5 (m, 4H), 3.4-3.4 (m, 1H), 3.3-3.1 (m, 2H), 2.9 (t, J=7.2 Hz, 3H), 2.7 (s, 4H), 2.4 (t, J=6.0 Hz, 2H), 2.3 (dt, J=14.4, 7.4 Hz, 4H), 1.9 (q, J=7.4 Hz, 2H).

¹³C NMR (101 MHz, MeOD) δ 175.4, 175.2, 174.1, 161.7, 154.2, 153.0, 152.6, 145.9, 144.7, 140.1, 137.0, 135.0, 130.9, 130.4, 129.7, 128.8, 124.4, 124.0, 117.5, 108.4, 71.6, 71.5, 71.5, 71.4, 71.3, 71.2, 70.5, 68.6, 68.3, 62.7, 58.1, 47.0, 43.9, 41.6, 40.4, 37.7, 36.3, 36.2, 36.1, 33.8, 23.2.

### i. 31: Azo-PEG[24]

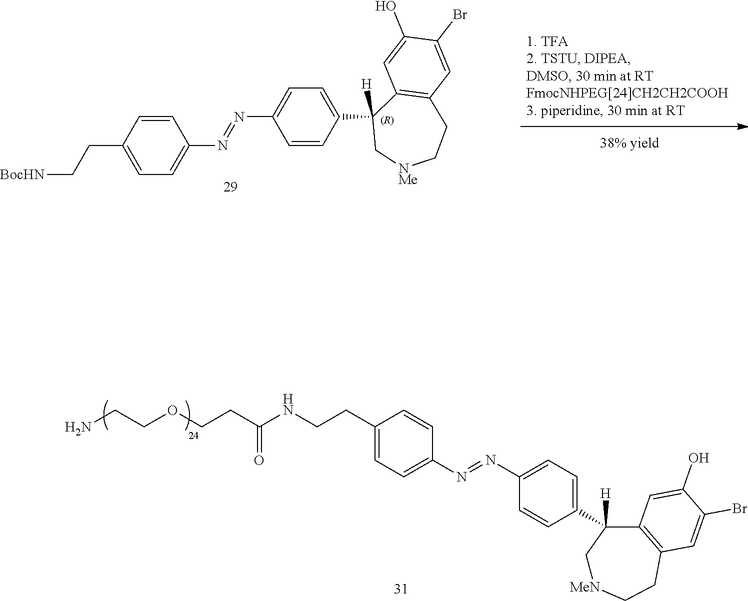

In a 1.5 mL vial under ambient conditions, FmocNHPEG[24]CH₂CH₂COOH (28.3 mg, 0.021 mmol, 1.2 eq) was dissolved in DMSO (133 uL). TSTU (6.2 mg, 0.021 mmol, 1.2 eq) and DIPEA (24 uL, 0.140 mmol, 8.1 eq) were added. The reaction was stirred for 30 min until full conversion was determined by LCMS analysis. In the meanwhile, in a 1.5 mL vial, 29 (10.0 mg, 0.017 mmol, 1.0 eq) was Boc-deprotected by treatment with TFA (100 uL). The TFA was removed after 30 seconds by evaporation under a nitrogen stream. The crude azo amine was dissolved in DMSO (98 uL) and added to the activated acid with an additional amount of DIPEA (6 uL, 0.035 mmol, 2.0 eq). The reaction was stirred for Ih, until full conversion was determined by LCMS. Piperidine (171 uL, 1.726 mmol, 100 eq) was added to the reaction mixture and stirring was continued until full conversion was determined by LCMS. The crude reaction mixture was cooled in an ice-water bath and treated with HOAc (119 uL, 2.071 mmol, 120 eq). The mixture was purified by RP-HPLC (10-50% MeCN in H₂O with 0.1% formic acid over 6 min, semi-preparative column, 9.0 mL/min flow rate, detection at 360 nm, t_{R}=4.300 min) to yield the product 31 after lyophilization as yellow oil in 38% yield (10.6 mg, 0.007 mmol).

LCMS (5-100% MeCN in H₂O with 0.1% formic acid over 5 min) t_{R}=2.847 min, 360 nm detection.

LRMS (ESI): calc. for C₇₆H₁₃₀BrN₅O₂₆²⁺ [M+2H]²⁺: 803.9; found 804.0.

HRMS (ESI): calc. for C₇₆H₁₃₀BrN₅O₂₆²⁺ [M+2H]²⁺: 803.9088; found 803.9061.

¹H NMR (400 MHz, MeOD) δ 8.1-7.8 (m, 4H), 7.5 (d, J=8.1 Hz, 4H), 7.4 (s, 1H), 6.3 (s, 1H), 4.7 (d, J=9.0 Hz, 1H), 3.8-3.8 (m, 2H), 3.7-3.6 (m, 96H), 3.6-3.5 (m, 3H), 3.4 (s, 1H), 3.2 (t, J=5.1 Hz, 2H), 3.1-2.9 (m, 5H), 2.9 (s, 3H), 2.4 (t, J=6.0 Hz, 2H).

¹³C NMR (101 MHz, MeOD) δ 174.0, 154.5, 153.2, 152.6, 144.8, 135.1, 132.3, 130.9, 130.5, 124.5, 124.0, 117.6, 108.8, 71.5, 71.5, 71.5, 71.4, 71.3, 71.3, 71.3, 71.3, 71.2, 71.2, 71.1, 71.0, 70.8, 70.5, 68.5, 68.2, 67.9, 61.7, 57.7, 47.2, 46.3, 41.6, 40.7, 40.2, 37.7, 36.3, 32.4.

IR (neat) 3462 (b), 2918 (m), 2872 (m), 1596 (m), 1457 (w), 1348 (w), 1249 (w). 1103 (s), 951 (w) cm⁻¹.

i. P-D1block(24)

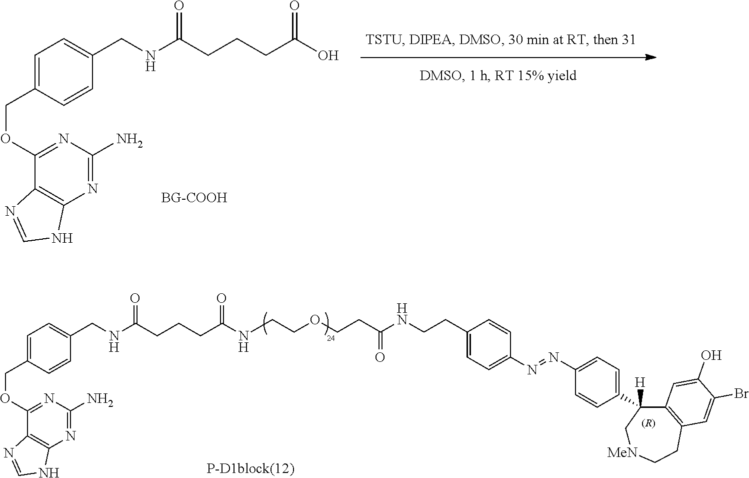

In a 1.5 mL vial, BG-COOH (3.8 mg, 9.9 umol, 1.5 eq) was dissolved in DMSO (36 uL). TSTU (3.0 mg, 9.9 umol, 1.5 eq) and DIPEA (5 uL, 26.4 umol, 4.0 eq) were added and the reaction mixture was stirred for 30 min, until full conversion was determined by LCMS analysis. 31 (10.6 mg, 6.6 umol, 1.0 eq) was added to the reaction mixture as a solution in DMSO (51 uL) and more DIPEA (2 uL, 13.2 umol, 2.0 eq) was added. After the reaction was judged complete by LCMS analysis, the mixture was purified by RP-HPLC (10-40% MeCN in H₂O with 0.1% formic acid over 9 min, semi-preparative column, 9.0 mL/min flow rate, detection at 360 nm, t_{R}=8.095 min) to yield the product after evaporation of the solvent at 40° C. in vacuo as a yellow oil in 15% yield (1.9 mg, 0.962 umol). P-D1block(24) was synthesized using starting materials of >99% ee.

LCMS (5-100% MeCN in H₂O with 0.1% formic acid over 5 min) t_{R}=3.082 min, 360 nm detection.

LRMS (ESI): calc. for C₉₄H₁₄₆BrN₁₁Na₂O₂₉²⁺ [M+2Na]²⁺: 1009.5; found 1009.4.

HRMS (ESI): calc. for C₉₄H₁₄₈BrN₁₁O₂₉²⁺ [M+2H]²⁺: 986.9808; found 986.9844.

¹H NMR (400 MHz, MeOD) δ 8.0-7.9 (m, 5H), 7.5-7.4 (m, 6H), 7.4-7.3 (m, 3H), 6.3 (s, 1H), 5.6 (s, 2H), 4.4 (d, J=8.7 Hz, 1H), 4.4 (s, 2H), 3.7 (d, J=6.0 Hz, 2H), 3.6-3.6 (m, 92H), 3.5-3.5 (m, 4H), 3.5-3.4 (m, 2H), 3.3-3.2 (m, 1H), 3.2-3.0 (m, 3H), 2.9 (t, J=7.1 Hz, 2H), 2.9-2.8 (m, 11H), 2.5-2.4 (m, 6H), 2.3 (dt, J=13.5, 7.5 Hz, 4H), 2.0-1.9 (m, 2H).

¹³C NMR (101 MHz, MeOD) δ 175.4, 175.2, 174.1, 161.6, 153.8, 152.8, 152.6, 146.9, 145.4, 144.6, 140.1, 137.0, 134.8, 134.3, 130.9, 130.5, 129.7, 128.8, 124.3, 124.0, 117.5, 108.0, 71.5, 71.5, 71.4, 71.3, 71.2, 70.5, 68.6, 68.3, 63.4, 58.4, 49.5, 47.5, 43.9, 41.6, 40.3, 37.7, 36.3, 36.2, 36.2, 34.8, 23.2.

IR (neat) 2871 (m), 1738 (w), 1625 (m), 1581 (m), 1455 (w), 1352 (m), 1281 (w), 1104 (s), 944 (m) cm⁻¹

### Results

Aniline intermediate 27 was prepared to set the stage for a Baeyer-Mills reaction for the installment of the azobenzene unit (FIG. 2): Commercial p-nitrophenacyl bromide was converted to the epoxide in excellent yield by reduction with NaBH₄, followed by treatment with base. Epoxide opening of 22 with 4-methoxyphenyl ethylamine installed racemic phenylephrine derivative 23 that was cyclized to benzazepine 24 with PPA at 100° C. In an Eschweiler-Clarke reaction, methylation of the secondary amine was achieved in good yield. Chiral resolution of 25-rac using a chiral tartaric acid derivative yielded biologically active R-enantiomer 25 in >99% ee, as determined by HPLC. The sign of the optical rotation matched literature reported values, and absolute configuration was assumed based on literature precedence. Bromination followed by Raney-Nickel reduction furnished desired aniline 27 in overall good yields that underwent the desired Baeyer-Mills reaction with prepared nitroso-phenyl ethyl Boc-amine. BBr₃ effected methoxy deprotection of 28 afforded desired phenol in addition to Boc-deprotection. The doubly deprotected material can be purified by RP-HPLC and taken on to the next step, however, proved to be unstable. The primary amine was re-protected with Boc₂O to yield Boc-protected product 29 that was stable and could be purified on SiO₂. In situ Boc-deprotection with TFA, followed by TSTU-mediated peptide coupling with FmocNHPEG[n]CH2CH2COOH of different lengths, yielded photoswitchable D1 antagonists with the desired long flexible linker. Piperidine effected Fmoc deprotection to unveil primary amines 30 (PEG[12]) and 31 (PEG[24]) were that was then coupled to the SNAP-tag substrate benzyl guanine by TSTU-mediated peptide coupling. Finally, desired remotely tethered D1 antagonist R-P-D1block(12) and R-P-D1block(24) were obtained. This procedure starting from 25 was performed using pure R-intermediate and using either PEG[12]- or PEG[24]-linkers. Additionally, a S-configured sample of 25 with 56% ee was used to generate S-P-D1block(12).

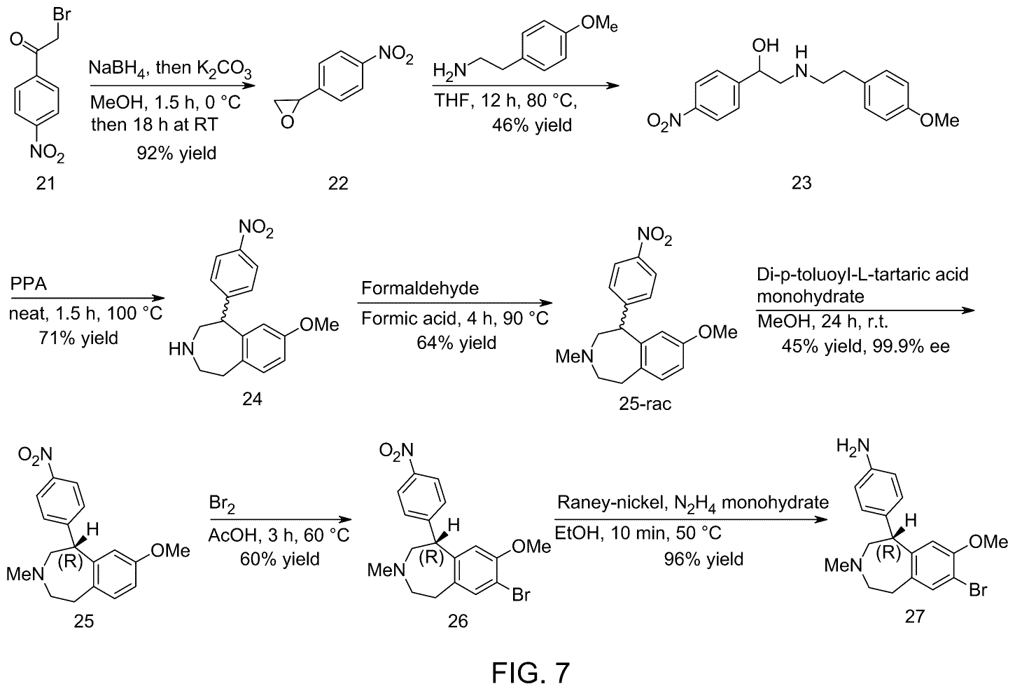

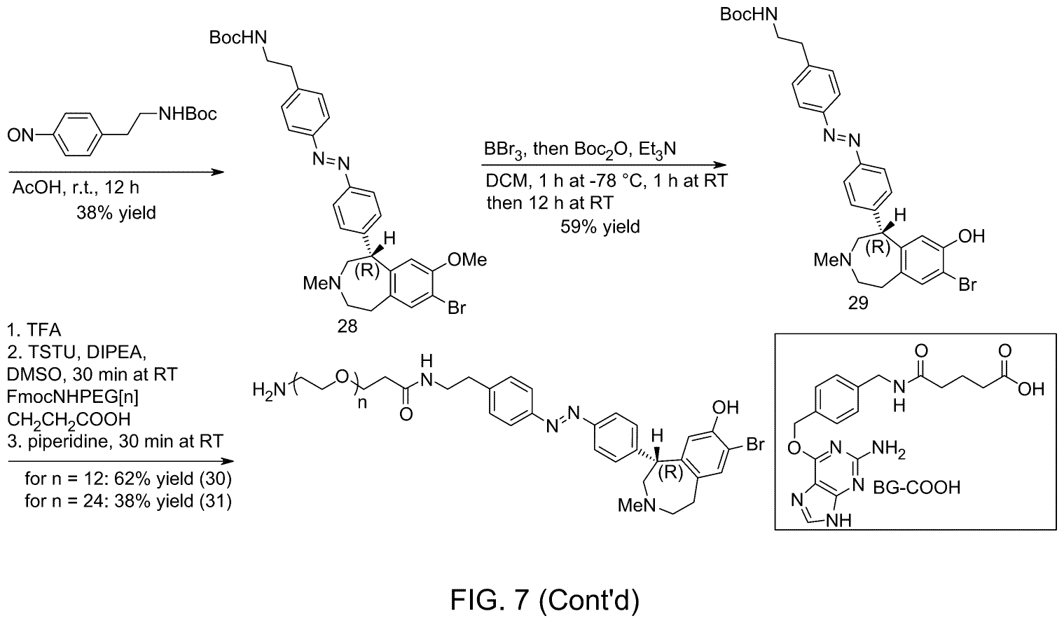

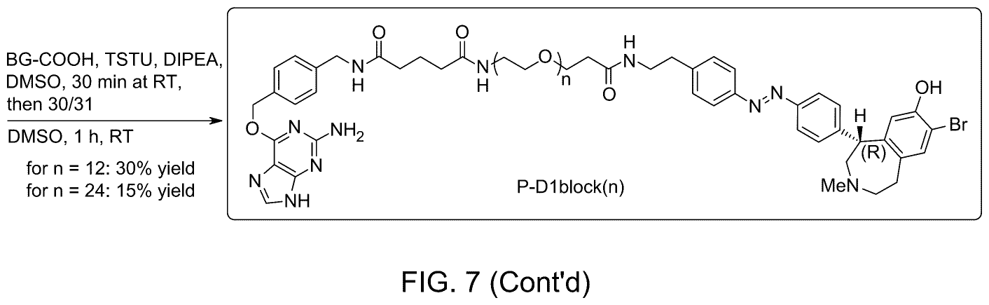

FIG. 7 depicts multistep chemical synthesis of P-D1block.

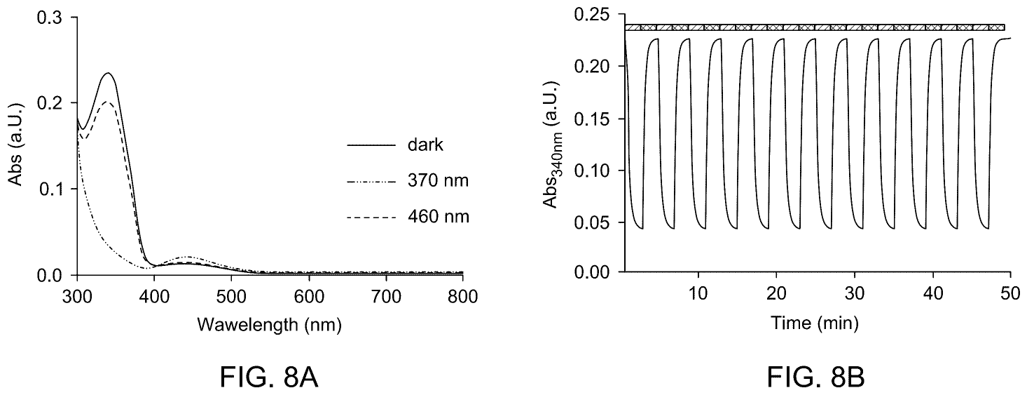

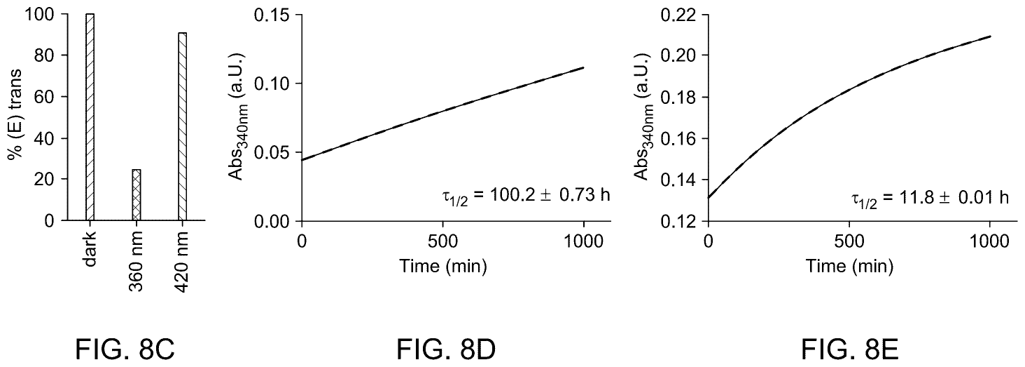

Photophysical characterization confirmed classic azobenzene properties (FIG. 8A-8E) of P-D1block(24). The photophysical properties for all other P-D1block(n)s are assumed to not be different, since no change in the azobenzene core occurs. P-D1block(24) is bistable and can be switched reversibly without fatigue using 370 nm/420 nm light irradiation. Under 360 nm, 25% trans-isomer remains, at 420 nm, 9% cis-isomer remains.

FIG. 8A-8E: Photophysical Characterization of P-D1block(24) A) UV Vis absorption spectra of dark-adapted (100% trans) P-D1block(24) in DMSO (20 μM), and after preirradiation with 370/460 nm for 10 min. B) Reversible switching of P-D1block(24) without fatigue in DMSO (20 μM) under 370/460 nm irradiation. C) Photostationary state (PSS) of P-D1block(24) in DMSO (20 μM, 24° C.), after preirradiation with 360/420 nm for 10 min. Detection of PSS was achieved by LCMS by separation of isomers and integration of Abs at the isosbestic point of cis- and trans-isomers at the respective elution solvent mixtures. D, E) Thermal relaxation of P-D1block(24) in 10% DMSO in PBS and 100% DMSO at 37° C., detection of Abs at 340 nm after pre-irradiation of the samples for 10 min at 360 nm.

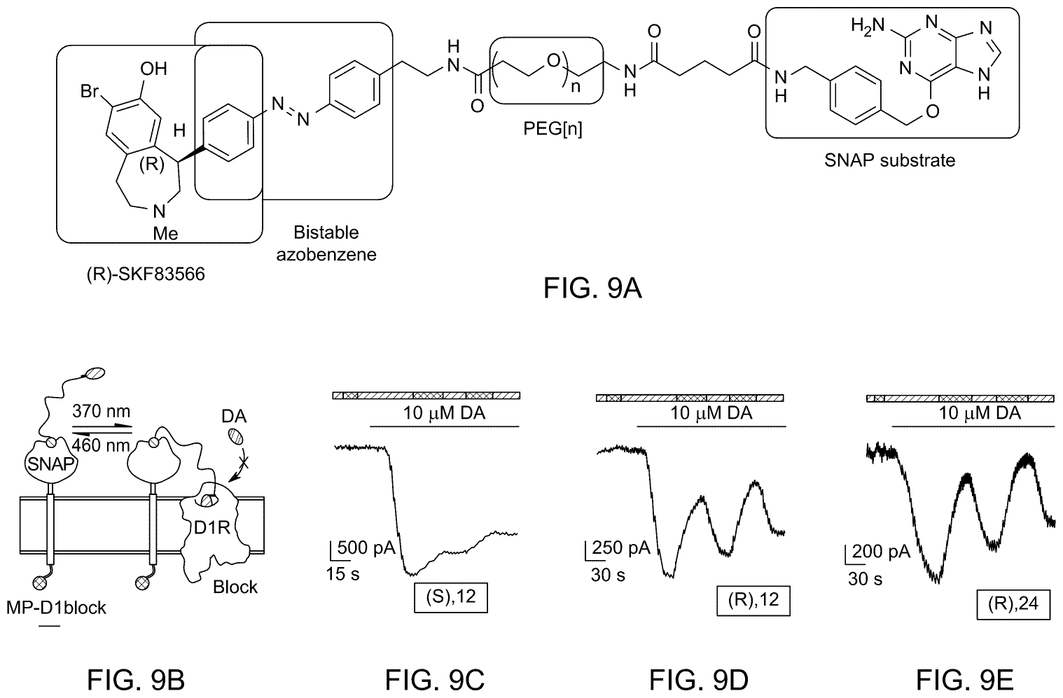

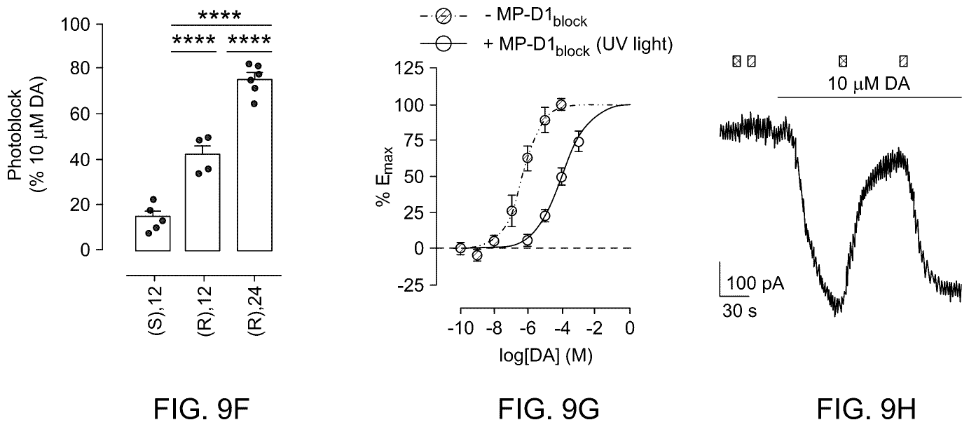

P-D1block (FIG. 9A) was tested in HEK293T cells, which co-expressed the membrane anchor (M), composed of SNAP fused to a single pass transmembrane segment, the D1R, a homotetramerizing mutant (F137S) of the GIRK1 channel, TdTomato to mark the expressing cells, and Gαᵢₛ₁₃ to couple D1R to the GIRK1 channel. Cells were tested using whole-cell patch clamp electrophysiology. Dopamine activation of D1R was monitored as inward GIRK current at high external [K⁺] at a negative holding potential and photo-antagonism measured as a block of GIRK current elicited by D1R activation by dopamine (FIG. 9B). The M-conjugated versions of the S and R enantiomers (FIG. 8) with a PEG linker length of 12 repeats [(S)-P-D1block(12) and (R)-P-D1block(12)] were tested. Stronger photo-block was observed with the R enantiomer (FIG. 9C, D, F). The R enantiomer with a longer (24 repeat) PEG chain was tested. (R)-P-D1block(24) elicited the most effective photo-antagonism, blocking ˜80% of the GIRK current elicited by saturating (10 μM) dopamine action (FIG. 9E, F), due to an ˜100-fold increase in apparent affinity of the photoswitched cis state (FIG. 9G). In absence of dopamine activation of D1R, photoswitching of (R)-P-D1block(24) had no effect, indicating that it acts as a pure photo-antagonist. In summary, this tool provides a powerful ability to temporally disable the physiological activation of D1Rs in specific cells and specific locations.

FIG. 9A-9H: Patch clamp electrophysiology of P-D1block in HEK293T cells. A) Chemical Structure of R-P-D1block(n). B) Schematic representation of MP-D1block activity: Upon formation of MP-D1block by covalent tagging of the M protein with P-D1block, the action of dopamine on the D1R can be blocked in a light-dependent manner upon irradiation of the photoswitch. C) P-D1block is virtually inactive in its S-configuration. The residual antagonism is likely due to only the remaining (R)-configured isomer in the sample, as it only had 56% ee (22% (R)-content). D, E, F, G) P-D1block(24) outperforms P-D1block(12) and blocks up to 80% of sat. dopamine reversibly under 360 nm irradiation. H) Due to the bistable nature of the photoswitch, pulsed light is sufficient to elicit reversible blocking of dopamine at the D1 receptor.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A system comprising:
a1) a conjugate of Formula I:
DRL-HP-L-HT (I)
wherein:
DRL is a dopamine receptor ligand;
HP is a hydrophobic moiety;
L is a linker; and
H is a halogen-containing substrate; and
b1) a fusion polypeptide, or a recombinant expression vector comprising a nucleotide sequence encoding the fusion polypeptide, wherein the fusion polypeptide comprises:
i) a self-labeling protein tag;
ii) a peptide linker; and
iii) a membrane-anchoring polypeptide; or
a2) a conjugate of Formula I:
DRL-HP-L-HT (I)
wherein:
DRL is a dopamine receptor ligand;
HP is a hydrophobic moiety;
L is a linker; and
H is a halogen-containing substrate; and
b2) a fusion polypeptide, or a recombinant expression vector comprising a nucleotide sequence encoding the fusion polypeptide, wherein the fusion polypeptide comprises:
i) a self-labeling protein tag; and
ii) an antibody specific for a D2 dopamine receptor.

2. The system of claim 1, wherein H comprises benzylguanine, chloroalkane, or benzylcytosine.

3. The system of claim 1 or claim 2, wherein DRL comprises an aminoindenyl moiety, a N-propyl 2-aminoindenyl moiety, phenylpiperazine moiety, a halo-substituted phenylpiperazine moiety, or a methoxy substituted phenylpiperazine moiety.

4. The system of any one of claims 1-3, wherein HP comprises a moiety selected from an azobenzene, a cyclic azobenzene, an azoheteroarene, a fulgide, a spiropyran, a triphenyl methane, a thioindigo, a diarylethene, and an overcrowded alkene.

5. The system of any one of claims 1-3, wherein HP comprises an azobenzene moiety.

6. The system of any one of claims 1-5, wherein DRL is a D2 dopamine receptor ligand.

7. The system of any one of claims 1-6, wherein the linker comprises a polyalkylene glycol.

8. The system of claim 7, wherein the polyalkylene glycol is poly(ethylene glycol) (PEG).

9. The system of claim 8, wherein the linker comprises (PEG)n, where n is an integer from 8 to 24.

10. The system of any one of claims 1-8, wherein HP comprises an azobenzene moiety that isomerizes in response to visible light.

11. The system of claim 6, wherein the DRL functions as a D2 dopamine receptor agonist.

12. The system of claim 6, wherein the DRL functions as a positive allosteric modulator of the D2 dopamine receptor.

13. The system of any one of claims 1-12, wherein the conjugate is a compound of Formula IA: 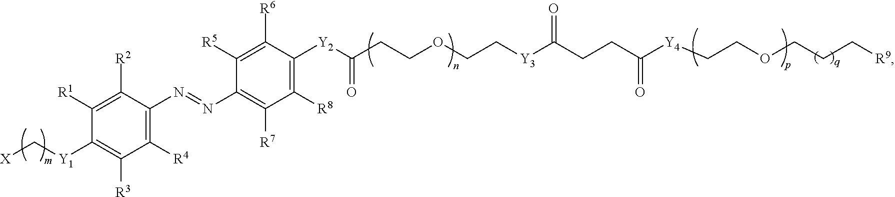
wherein X is: 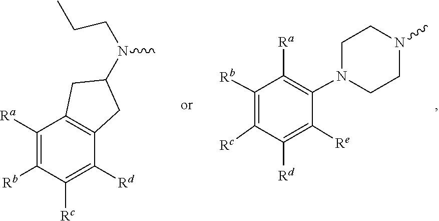
wherein [symbol] represents the X—(C)ₘ— bond;
m is an integer from 1-10;
n is an integer from 1-50;
p is an integer from 1-10;
q is an integer from 1-25;
Y₁, Y₂, Y₃ and Y₄ are independently selected from C, N, O or S;
R^{a}, R^{b}, R^{c} and R^{d} are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
R¹, R², R³, R⁴, R⁵, R⁶, R⁷ and R⁸ are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
R⁹ is selected from hydrogen, halogen, alkoxy, nitro, amino, hydroxy, cyano, thiol.

14. The system of claim 13, wherein:
a) one or more of R^{a}, R^{b}, R^{c} and R^{d} is independently selected from F, Cl, Br and I; or
b) one or more of R^{a}, R^{b}, R^{c} and R^{d} is a C(1-6) alkoxy, such as methoxy or ethoxy.

15. The system of any one of claims 1-14, wherein:
a) the conjugate is a compound of Formula 1A: 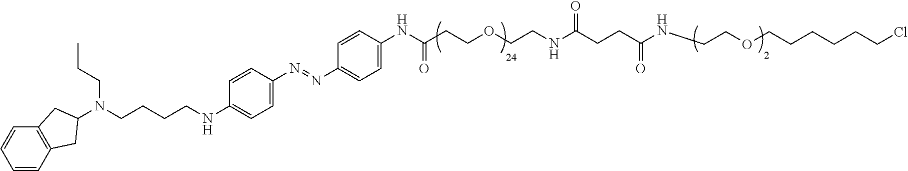
b) the conjugate is a compound of Formula 1B: 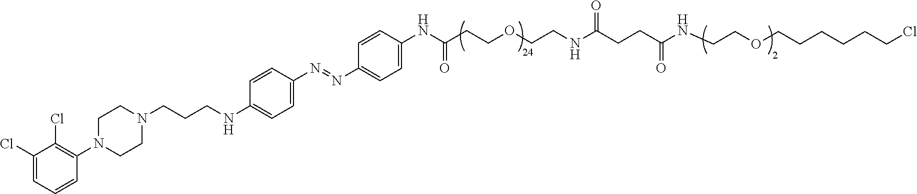
or
c) the conjugate is a compound of Formula 1C:

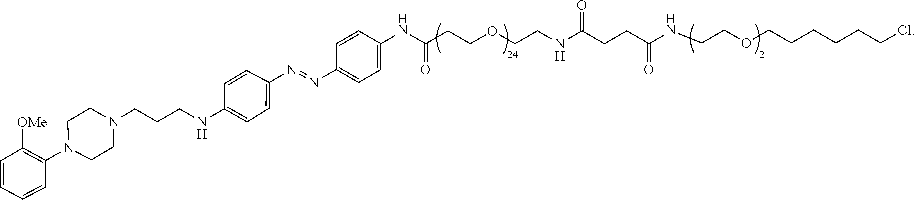

16. The system of any one of claims 1-14, wherein the conjugate is a compound of Formula IIA: 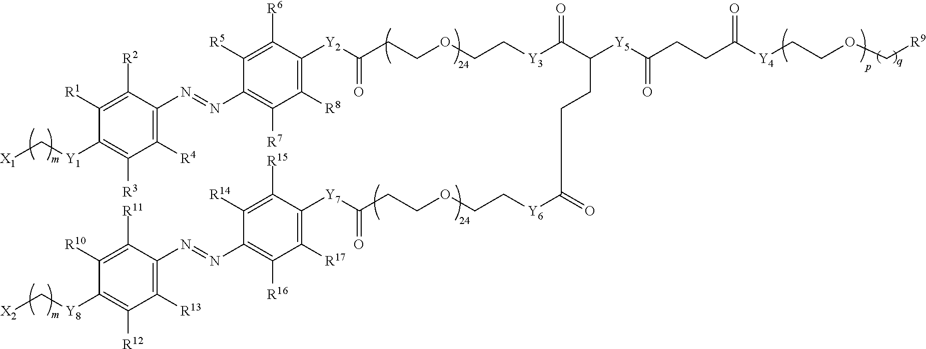
wherein X₁ and X₂ are independently selected from: 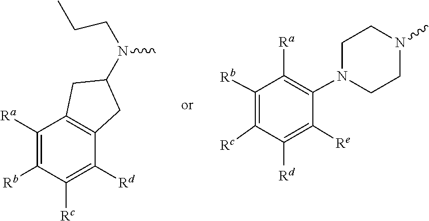
wherein [symbol] represents the X—(C)ₘ— bond;
m is an integer from 1-10;
n is an integer from 1-50;
p is an integer from 1-10;
q is an integer from 1-25;
Y₁, Y₂, Y₃, Y₄, Y₅, Y₆, Y₇ and Y₈ are independently selected from C, N, O or S;
R^{a}, R^{b}, R^{c} and R^{d} are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
R¹, R², R³, R⁴, R⁵, R⁶, R⁷, R⁸, R¹⁰, R¹¹, R¹², R¹³, R¹⁴, R¹⁵, R¹⁶ and R¹⁷ are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
R⁹ is selected from hydrogen, halogen, alkoxy, nitro, amino, hydroxy, cyano, thiol.

17. The system of claim 16, wherein:
a) one or more of R^{a}, R^{b}, R^{c} and R^{d} is independently selected from F, Cl, Br and I; or
b) one or more of R^{a}, R^{b}, R^{c} and R^{d} is a C(1-6) alkoxy, such as methoxy or ethoxy.

18. The system of claim 17, wherein the conjugate is a compound of Formula 2A:

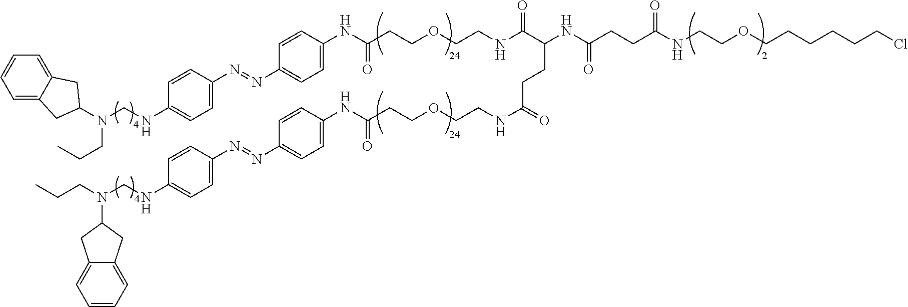

19. The system of claim 1, wherein the DRL is a D2 dopamine receptor antagonist.

20. The system of claim 19, wherein the conjugate has the following structure:

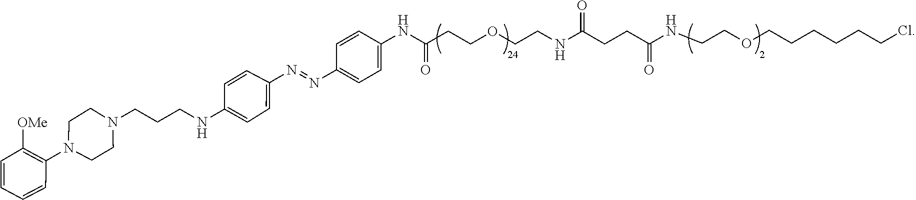

21. The system of claim 19, wherein the conjugate has the following structure:

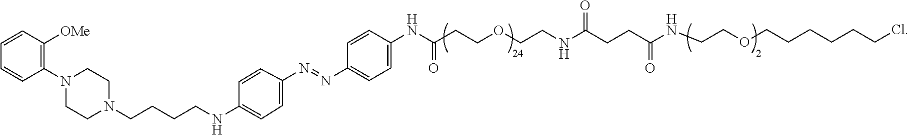

22. The system of claim 1, wherein the DRL is a D1 dopamine receptor ligand.

23. The system of claim 1, wherein the DRL is a D1 dopamine receptor antagonist.

24. The system of claim 23, wherein the conjugate has the following structure:

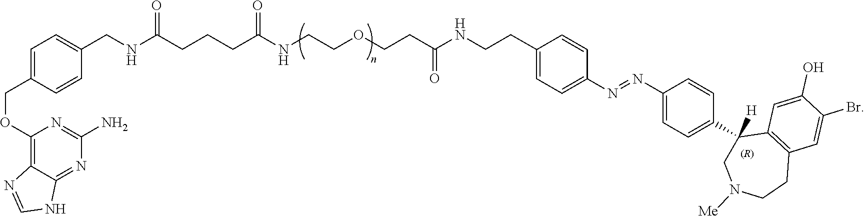

25. The system of any one of claims 1-24, wherein the self-labeling protein tag comprises:
a) an amino acid sequence having at least 80% amino acid sequence identity to the SNAP polypeptide amino acid sequence set forth in SEQ ID NO:1;
b) an amino acid sequence having at least 80% amino acid sequence identity to the CLIP polypeptide amino acid sequence set forth in SEQ ID NO:2; or
c) an amino acid sequence having at least 80% amino acid sequence identity to the HALO polypeptide amino acid sequence set forth in SEQ ID NO:3.

26. The system of any one of claims 1-25, wherein the fusion polypeptide comprises an endoplasmic reticulum (ER) export signal peptide.

27. The system of any one of claims 1-26, wherein the peptide linker comprises the amino acid sequence EAAAK (SEQ ID NO:13).

28. The system of any one of claims 1-27, wherein the nucleotide sequence encoding the fusion polypeptide is operably linked to a cell type-specific promoter.

29. The system of claim 28, wherein the promoter is a dopamine-1 receptor promoter.

30. The system of any one of claims 1-29, where the antibody is a nanobody or a single-chain Fv.

31. A method of modulating the activity of a D2 dopamine receptor, the method comprising:
a) contacting a cell comprising the D2 dopamine receptor with a system of any one of claims 1-30; and
b) exposing the cell to light of a wavelength that isomerizes the photoisomerizable agent present in the conjugate.

32. The method of claim 31, wherein the cell is in an individual.

33. The method of claim 31 or claim 32, wherein the light is provided by an implantable light source.

34. The method of any one of claims 31-33, wherein the cell is a direct pathway medium spiny neuron.

35. A method of treating Parkinson's disease in an individual, the method comprising administering the system of any one of claims 1-30 into the dorsal striatum of the individual.

36. The method of claim 35, comprising exposing the dorsal striatum to light of a wavelength that isomerizes the photoisomerizable agent present in the conjugate.

37. The method of claim 35 or 36, wherein the light is provided by an implantable light source.

38. A compound of Formula I:
DRL-HP-L-HT (I)
wherein:
DRL is a dopamine receptor ligand;
HP is a hydrophobic moiety;
L is a linker; and
H is a halogen-containing substrate.

39. The compound of claim 38, wherein H comprises benzylguanine, chloroalkane, or benzylcytosine.

40. The compound of claim 38 or claim 39, wherein DRL comprises an aminoindenyl moiety, a N-propyl 2-aminoindenyl moiety, phenylpiperazine moiety, a halo-substituted phenylpiperazine moiety, or a methoxy substituted phenylpiperazine moiety.

41. The compound of any one of claims 38-40, wherein HP comprises a moiety selected from an azobenzene, a cyclic azobenzene, an azoheteroarene, a fulgide, a spiropyran, a triphenyl methane, a thioindigo, a diarylethene, and an overcrowded alkene.

42. The compound of any one of claims 38-41, wherein HP comprises an azobenzene moiety.

43. The compound of any one of claims 38-42, wherein DRL is a D2 dopamine receptor ligand.

44. The compound of any one of claims 38-43, wherein the linker comprises a polyalkylene glycol.

45. The compound of claim 44, wherein the polyalkylene glycol is poly(ethylene glycol) (PEG).

46. The compound of claim 45, wherein the linker comprises (PEG)n, where n is an integer from 8 to 24.

47. The compound of any one of claims 38-46, wherein HP comprises an azobenzene moiety that isomerizes in response to visible light.

48. The compound of claim 43, wherein the DRL functions as a D2 dopamine receptor agonist.

49. The compound of claim 43, wherein the DRL functions as a positive allosteric modulator of the D2 dopamine receptor.

50. The compound of any one of claims 38-49, wherein the compound is of Formula IA: 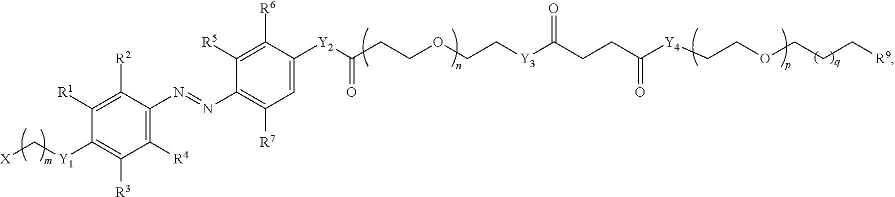
wherein X is: 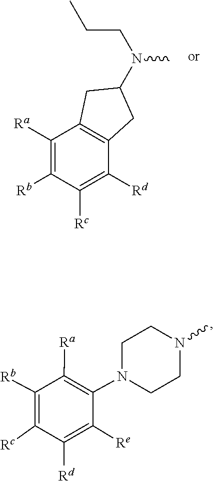
wherein [symbol] represents the X—(C)ₘ— bond;
m is an integer from 1-10;
n is an integer from 1-50;
p is an integer from 1-10;
q is an integer from 1-25;
Y₁, Y₂, Y₃ and Y₄ are independently selected from C, N, O or S;
R^{a}, R^{b}, R^{c} and R^{d} are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
R¹, R², R³, R⁴, R⁵, R⁶, R⁷ and R⁸ are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
R⁹ is selected from hydrogen, halogen, alkoxy, nitro, amino, hydroxy, cyano, thiol.

51. The compound of claim 50, wherein:
a) one or more of R^{a}, R^{b}, R^{c} and R^{d} is independently selected from F, Cl, Br and I; or
b) one or more of R^{a}, R^{b}, R^{c} and R^{d} is a C(1-6) alkoxy, such as methoxy or ethoxy.

52. The compound of any one of claims 38-51, wherein:
a) the conjugate is a compound of Formula 1A: 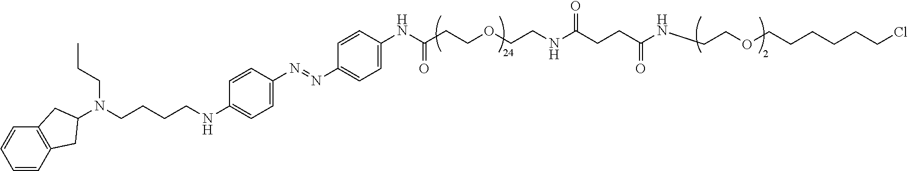
b) the conjugate is a compound of Formula 1B: 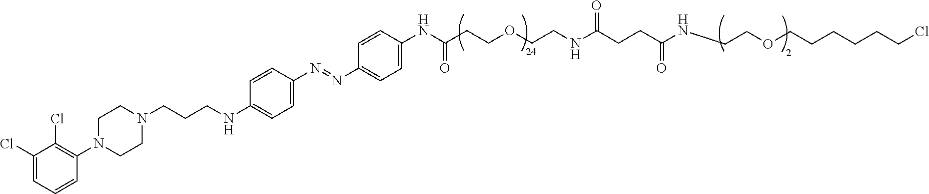
or
c) the conjugate is a compound of Formula 1C:

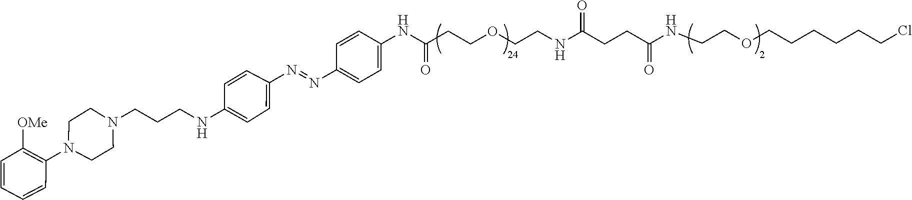

53. The compound of any one of claims 38-49, wherein the conjugate is a compound of Formula IIA: 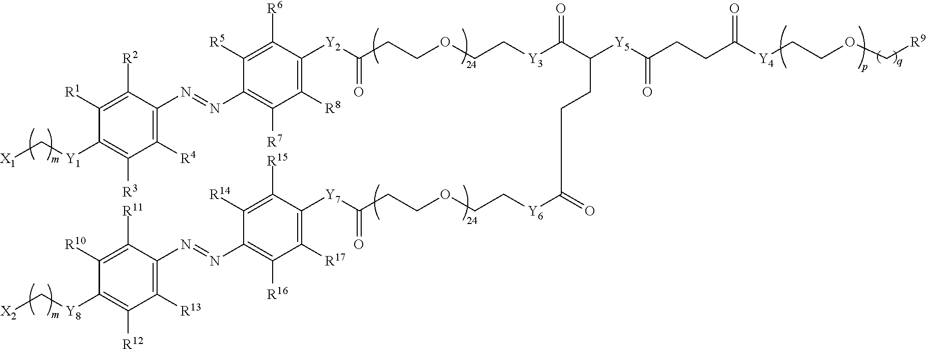
wherein X₁ and X₂ are independently selected from: 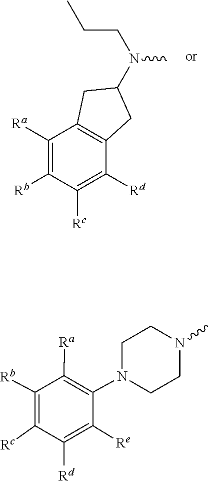
wherein [symbol] represents the X—(C)ₘ— bond;
m is an integer from 1-10;
n is an integer from 1-50;
p is an integer from 1-10;
q is an integer from 1-25;
Y₁, Y₂, Y₃, Y₄, Y₅, Y₆, Y₇ and Y₈ are independently selected from C, N, O or S;
R^{a}, R^{b}, R^{c} and R^{d} are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
R¹, R², R³, R⁴, R⁵, R⁶, R⁷, R⁸, R¹⁰, R¹¹, R¹², R¹³, R¹⁴, R¹⁵, R¹⁶ and R¹⁷ are independently selected from hydrogen, halogen, alkoxy, nitro, amino, hydroxy, cyano, thiol, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
R⁹ is selected from hydrogen, halogen, alkoxy, nitro, amino, hydroxy, cyano, thiol.

54. The compound of claim 53, wherein:
a) one or more of R^{a}, R^{b}, R^{c} and R^{d} is independently selected from F, Cl, Br and I; or
b) one or more of R^{a}, R^{b}, R^{c} and R^{d} is a C(1-6) alkoxy, such as methoxy or ethoxy.

55. The compound of claim 54, wherein the conjugate is a compound of Formula 2A:

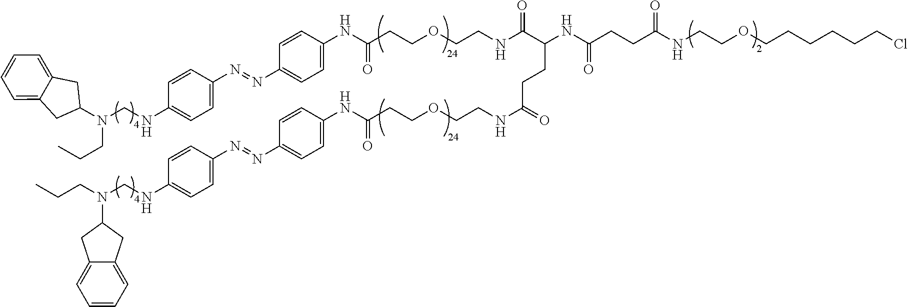

56. The compound of claim 38, wherein the DRL is a D2 dopamine receptor antagonist.

57. The compound of claim 56, wherein the conjugate has the following structure:

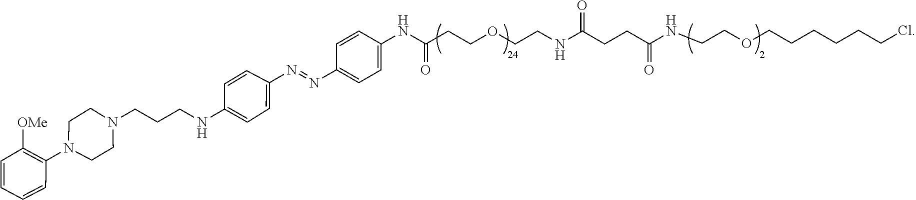

58. The compound of claim 56, wherein the conjugate has the following structure:

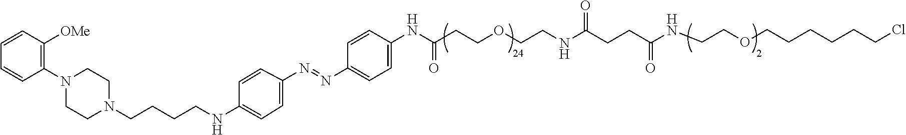

59. The compound of claim 38, wherein the DRL is a D1 dopamine receptor antagonist.

60. The compound of claim 59, wherein the conjugate has the following structure:

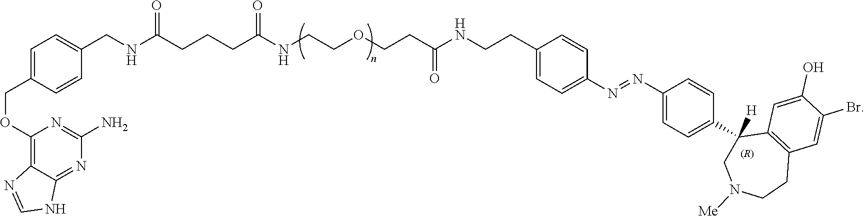

61. A method of treating Parkinson's disease in an individual, the method comprising administering the conjugate of any one of claims 38-60 into the dorsal striatum of the individual.

62. The method of claim 61, comprising exposing the dorsal striatum to light of a wavelength that isomerizes the photoisomerizable agent present in the conjugate.

63. The method of claim 61 or 62, wherein the light is provided by an implantable light source.


SEQUENCE LISTING (13 sequences)

SEQ ID NO: 1 - protein, 182 aa, synthetic construct
MDKDCEMKRTTLDSPLGKLELSGCEQGLHRIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLG

SEQ ID NO: 2 - protein, 182 aa, synthetic construct
MDKDCEMKRTTLDSPLGKLELSGCEQGLHRIIFLGKGTSAADAVEVPAPAAVLGGPEPLIQATAWLNAYFHQPEAIEEFPVPALHHPVFQQESFTRQVLWKLLKVVKFGEVISESHLAALVGNPAATAAVNTALDGNPVPILIPCHRVVQGDSDVGPYLGGLAVKEWLLAHEGHRLGKPGLG

SEQ ID NO: 3 - protein, 297 aa, synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 4 - skipped

SEQ ID NO: 5 - protein, 5 aa, synthetic construct
VKESL

SEQ ID NO: 6 - protein, 5 aa, synthetic construct
VLGSL

SEQ ID NO: 7 - protein, 16 aa, synthetic construct
NANSFCYENEVALTSK

SEQ ID NO: 8 - protein, 6 aa, synthetic construct
  VARIANT 2: note = X is any amino acid
FXYENE

SEQ ID NO: 9 - protein, 7 aa, synthetic construct
FCYENEV

SEQ ID NO: 10 - protein, 44 aa, synthetic construct
  VARIANT 2..21: note = Wherein up to 2 copies of EAAAK may be deleted
  VARIANT 24..43: note = Wherein up to 2 copies of EAAAK may be deleted
  VARIANT 23..44: note = Wherein AEAAAKEAAAKEAAAKEAAAKA may be deleted
AEAAAKEAAAKEAAAKEAAAKAAEAAAKEAAAKEAAAKEAAAKA

SEQ ID NO: 11 - protein, 20 aa, synthetic construct
  VARIANT 1..20: note = Wherein up to 9 copies of XP may be deleted
  VARIANT 1: note = X is A, K, or E
  VARIANT 3: note = X is A, K, or E
  VARIANT 5: note = X is A, K, or E
  VARIANT 7: note = X is A, K, or E
  VARIANT 9: note = X is A, K, or E
  VARIANT 11: note = X is A, K, or E
  VARIANT 13: note = X is A, K, or E
  VARIANT 15: note = X is A, K, or E
  VARIANT 17: note = X is A, K, or E
  VARIANT 19: note = X is A, K, or E
XPXPXPXPXPXPXPXPXPXP

SEQ ID NO: 12 - protein, 30 aa, synthetic construct
  VARIANT 1..30: note = Wherein up to 5 copies of EAAAK may be deleted
EAAAKEAAAKEAAAKEAAAKEAAAKEAAAK

SEQ ID NO: 13 - protein, 5 aa, synthetic construct
EAAAK